(19) United States
(12) Patent Application Publication
(10) Pub. No.: US 2016/0331814 A1
(43) Pub. Date: Nov. 17, 2016

(54) Col 11A1 AND NOVEL FRAGMENT THEREOF FOR REGULATION OF BONE MINERALIZATION

(71) Applicant: BOISE STATE UNIVERSITY, Boise, ID (US)

(72) Inventor: Julia Thom Oxford, Boise, ID (US)

(21) Appl. No.: 15/131,544

(22) Filed: Apr. 18, 2016

Related U.S. Application Data
(60) Provisional application No. 62/152,079, filed on Apr. 24, 2015.

(51) Int. Cl. A61K 38/39; C07K 14/78; C12N 15/86; A61K 48/00

(52) U.S. Cl. CPC A61K 38/39; A61K 48/005; C07K 14/78; C12N 15/86

(57) ABSTRACT

The present invention relates to the discovery that the collagen 11a1 (Col 11A1) protein, and/or fragments thereof, may be used to modulate bone mineralization. In some embodiments, bone mineralization is promoted by the addition of Col 11A1 or a fragment thereof, by pharmaceutical compositions that increase the presence of Col 11A1, and in some embodiments, bone mineralization may desired to be inhibited by pharmaceutical compositions that interfere, impede, or inhibit Col 11A1. The invention includes compositions including a Col 11A1 polypeptide, or fragment and compositions including a nucleic acid that encodes a Col 11A1 polypeptide or fragment. The invention also provides methods and kits for using such polypeptides and nucleic acids to treat bone mineralization disorders, and promote bone growth and fracture healing.

### CROSS-REFERENCED TO RELATED APPLICATIONS

This application claims priority under 35 U.S.C. §119 to provisional application Ser. No. 62/152,079 filed Apr. 24, 2015 herein incorporated by reference in its entirety.

### GRANT REFERENCE

This invention was made with government support under Grants R01AR047985, K02AR48672, P20RR16454, P20GM103408, P20GM109095, and R15HD HD059949 from the National Institutes of Health (NIH). The Government has certain rights in the invention.

### FIELD OF THE INVENTION

The present invention is directed to nucleic acid sequences and proteins involved in bone mineralization and the modulation of the same for bone healing, regeneration and development.

### BACKGROUND OF THE INVENTION

In both Europe and the United States an estimated 5 to 6 million people sustain bone fractures each year due to trauma, sports- or activity-related injuries or osteoporosis. Most of these injuries may be treated with manual reduction and external fixation (e.g. a cast). Approximately 20 to 25% of fractures require hospitalization, usually with open surgical procedures.

Bone fracture is a condition where a physiological continuity of bone tissue is partially or completely broken off and generally classified on the basis of the outbreak mechanism into (a) fracture by external force, (b) pathological fracture, and (c) fatigue fracture. In addition, the state of bone fracture is classified on the basis of the fracture line (the line tracing the epiphysis generated by bone transection), into fissure fracture, greenstick fracture, transverse fracture, oblique fracture, spiral fracture, segmental fracture, comminuted fracture, avulsion fracture, compression fracture, depression fracture, and the like.

The primary goal of fracture treatment is sound union and the restoration of bone function without an outcome of deformity. Obtaining these goals quickly is an increasingly important concern due to disability issues and cost-containment. In a significant part of the patient population both goals, i.e. sound union and fast restoration of bone function, are at risk due to the patient's age and/or general health condition, and/or the type and/or location of fracture. In particular, in case of osteoporotic patients, the risk of non-union and increased healing time is high. Osteoporosis is characterized by low bone mass and the structural deterioration of bone tissue leading to increased bone fragility, increased healing times and the occurrence of non-union. Delayed or incomplete healing can be observed in approximately 5-10% of patients following a fracture of the long bones.

Only limited knowledge is available about the mechanisms behind poor healing. There is growing evidence suggesting a key role of inflammation and T-cell response within the bone repair processes following injury, wherein the T-cell response affects processes such as chemotaxis, recruitment of further immune and mesenchymal cells resulting in stimulating angiogenesis, and finally, enhancement of extracellular matrix synthesis (Schmidt-Bleek et al., J Orthop Res.; 27(9):1147-51; Kolar et al., Tissue Eng Part B Rev.; 16(4):427-34; Toben et al. J Bone Miner Res., January; 26(1):113-24).

Any new technique to stimulate bone repair or cartilage repair would be a valuable tool in treating bone fractures. A significant portion of fractured bones are still treated by casting, allowing natural mechanisms to effect wound repair. Although there have been advances in fracture treatment in recent years, including improved devices, the development of new processes to stimulate, or complement, the wound repair mechanisms would represent significant progress in this area. For example, efforts to influence bone repair using bone stimulating proteins and peptides, e.g., bone morphogenic proteins (BMPs), has resulted in only limited success.

As can be seen, there is a need for agents and compositions for modulating bone mineralization for bone regeneration and repair.

### SUMMARY OF THE INVENTION

The present invention relates to the discovery that collagen 11a1 (Col 11A1) protein, and/or fragments thereof, may be used to modulate bone mineralization. In some embodiments, bone mineralization is promoted by the addition of Col 11A1 or a fragment thereof, or by agents and pharmaceutical compositions that increase the presence or activity of Col 11A1, or through domain-specific expression in Col 11a1, and in some embodiments, bone mineralization may be inhibited by the addition of Col 11A1 or a fragment thereof, or by pharmaceutical compositions that include agents that interfere, impede, or inhibit activity or domain-specific expression in Col 11A1.

Accordingly, the invention includes compositions including a Col 11A1 polypeptide, or fragment thereof and compositions including a nucleic acid that encodes a Col 11A1 polypeptide or fragment. The invention also provides methods and kits for using such polypeptides and nucleic acids to treat bone mineralization disorders, and to promote bone growth and wound healing. The disease or condition may be, for instance, osteoporosis, juvenile osteoporosis, bone loss due to/or associated with the onset of menopause, osteoporotic fractures, giant cell tumors of bone, renal osteodystrophy, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, metastatic bone diseases or malignancy-induced osteoporosis and bone lysis, childhood idiopathic bone loss, periodontal bone loss, age-related loss of bone mass, osteotomy and bone loss associated with prosthetic ingrowth, other forms of osteopenia, and in other conditions where facilitation of bone repair or replacement is desired such as bone fractures, bone defects, plastic surgery, dental and other implantations.

Accordingly, in one aspect, the invention features a method of treating a bone mineralization disorder, or to treat a subject in need of bone growth or regeneration such as after bone fracture or injury, by increasing the amount or activity of Col 11A1 in the subject.

One method includes administering to the subject a therapeutically effective amount of a pharmaceutical composition including: (a) a Col 11A1 polypeptide or a fragment thereof; and (b) a pharmaceutically acceptable carrier.

In yet another aspect, the invention features a method of treating a bone mineralization disorder, or to promote bone growth or regeneration such as after injury in a subject, the method including administering to the subject a therapeutically effective amount of a pharmaceutical composition including: (a) an isolated nucleic acid encoding a Col 11A1 protein or a fragment thereof and (b) a pharmaceutically acceptable carrier.

Yet another aspect includes administering to the subject a therapeutically effective amount of a pharmaceutical composition including: (a) a compound which increases the activity of Col 11A1 in said subject; and (b) a pharmaceutically acceptable carrier.

In any embodiment of the invention, the polypeptide may be a Col 11A1 fragment as provided in SEQ ID NO:1 or a nucleic acid sequence encoding the same.

In any embodiment, the amino acid sequence or the polypeptide optionally includes a Col 11A1 polypeptide such as those of SEQ ID NO:1, 2, or 3, or a polypeptide that is at least 90% identical thereto, or to a fragment thereof, including the option that additional otherwise identical amino acids are replaced by conservative substitutions and further preferably including fusion proteins or other modifications such that the proteins are not naturally occurring.

In any embodiment, the polypeptide optionally is pegylated or glycosylated. In any embodiment, the pharmaceutical composition optionally includes a dimer of the polypeptide.

In any embodiment, the invention also includes a polynucleotide selected from the group consisting of:
(a) SEQ ID NO:4, 5, or 6 or a fragment thereof;
(b) the complement of any sequence in (a);
(c) a polynucleotide that hybridizes with a sequence of (a) or (b) under stringent conditions defined as hybridizing to filter bound DNA in 0.5M NaHPO₄, 7% SDS, 1 mM EDTA at 65° C., and washing in 0.1×SSC/0.1% SDS at 68° C.
(d) a polynucleotide that is at least 70% identical to the polynucleotide of (a) or (b);
(e) a polynucleotide that is at least 80% identical to the polynucleotide of (a) or (b);
(f) a polynucleotide that is at least 90% identical to the polynucleotide of (a) or (b); and
(g) a polynucleotide that is at least 95% identical to the polynucleotide of (a) or (b),
   preferably in combination with a second heterologous sequence.

In a further embodiment, the pharmaceutically acceptable carrier optionally includes saline, in yet another embodiment, the pharmaceutical composition is optionally lyophilized.

In any of the methods of the invention, the pharmaceutical composition is optionally administered subcutaneously, intravenously, orally, nasally, intramuscularly, sublingually, intrathecally, or intradermally. In certain embodiments, the pharmaceutical composition is administered subcutaneously. For example, the methods of the invention may optionally include administering a pharmaceutical composition including the polypeptides of the invention to the subject in a dosage of about 0.5 mg/kg/day to about 10 mg/kg/day (e.g., about 2 mg/kg/day to about 3 mg/kg/day). In other examples, the methods include administering the pharmaceutical composition to the subject between one and seven times a week (e.g., three times a week).

Any of the pharmaceutical compositions of the invention featuring an isolated nucleic acid may optionally include a recombinant expression vector (e.g., a lentiviral vector) including the isolated nucleic acid. In some embodiments, the isolated nucleic acid is optionally administered to the subject at a dosage of from about 0.1 mg to about 10 mg. The invention also features an isolated recombinant host cell transformed or transfected with such a vector.

The invention also features methods of producing any polypeptide of the invention, including culturing such a host cell in a culture medium under conditions suitable to effect expression of the polypeptide and recovering the polypeptide from the culture medium. For example, the host cell is optionally an L cell, a C127 cell, a 3T3 cell, a CHO cell, a BHK cell, or a COS-7 cell. In some embodiments, the host cell is a CHO cell (e.g., a CHO-DG44 cell).

The invention also features kits. For example, the invention features a kit including: (a) any of the pharmaceutical compositions of the invention and (b) instructions for administering the pharmaceutical composition to a subject to treat a bone mineralization disorder or a person in need of bone development/mineralization.

The present invention relates to Col 11A1 nucleic acid sequences and amino acid sequences. Additionally, the present invention relates to control over the influencing of bone mineralization and the bone mineralization pathway using the above nucleic acid sequences and amino sequences.

Additionally, the present invention relates to molecular tools developed from the nucleic acids and polypeptides including vectors, transfected host cells, transfected organisms knockout organisms, antibodies, hybridomas cells, Fab fragments, and homologous nucleic acid sequences and polypeptides.

As discussed herein, nucleic acid sequences and nucleic acid molecules will be used interchangeably. The isolated nucleic acid sequences include gDNAs, cDNAs, and a variety of other nucleic acid sequence fragments. It is contemplated that any of a variety of nucleic acid sequences can be used herein including genes, mRNA, cDNA, gDNA, tRNA, oligonucleotides, polynucleotides, and nucleic acid sequence fragments. As such, any nucleic acid sequence which expresses a polypeptide that influences bone mineralization is contemplated as part of the present invention, as well as mutant versions thereof. The nucleic acid sequences will include genes which are any hereditary unit that has an effect on the phenotype of an organism and can be transcribed into mRNAs which result in polypeptides, as well as rRNAs or tRNA molecules and regulatory genes. Also, alleles and mutant alleles and protein fragments are part of the definition of a gene as used herein.

Probes which hybridize to either nucleic acid sequences or the fragment encoding nucleic acid sequences are part of the present invention. The probes will include any of a variety of labels and can be either cDNA or RNA probes. The probes can be used to form a kit or similar tool for use in detecting the presence or absence of a particular Col 11A1 nucleic acid or polypeptide.

In certain embodiments the pharmaceutical composition of the invention include inhibitory molecules based upon the polypeptide and nucleic acid sequences disclosed herein, such as antibodies which bind to at least one of the previously mentioned amino acid sequences are used herewith. For example, the antibodies include monoclonal, polyclonal, recombinant, and antibody fragments. Any of a variety of antibodies may be used that bind to either Col 11A1 or the fragments disclosed here. The antibodies are designed to bind to the selected polypeptide and prevent it from binding to its normal antigen. Conversely, the antibodies can be designed such that they attack and destroy the chosen or selected polypeptides. Hybridomas can be formed which are used to produce the desired antibodies. As such any of a variety of cells can be used to produce both the polypeptides as well as the antibodies. Additional inhibition molecular tools may be used such as antisense molecules, RNAi molecules and the like.

In the context of the present invention, “Col 11A1 inhibitor” is understood as a compound inhibiting or reducing Col 11A1 protein expression and/or activity, or inhibiting the bone mineralization effects of Col 11A1 protein expression induction, and includes any compound that is capable of preventing or blocking Col 11A1 isoform-specific gene transcription and/or translation (i.e., modulating splicing or preventing or blocking said gene expression of exon 6A and/or exon 8 containing Col 11A1), or that is capable of preventing the protein encoded by said Col 11A1 gene from performing its desired function (increased bone mineralization activity); i.e., said term “Col 11A1 inhibitor” includes compounds acting either at the RNA level (e.g., antisense oligonucleotides (“antisense”), shRNA, siRNA, etc.), or at the protein level (e.g., antibodies, peptides, small organic compounds or small molecules, etc.).

By way of non-limiting illustration, Col 11A1 inhibitors include Col 11A1 gene expression inhibitory agents suitable for use in the present invention, and include, for example, antisense oligonucleotides specific for the gene, specific microRNAs, catalytic RNAs or specific ribozymes, specific interfering RNAs (siRNAs), RNAs with decoy activity, i.e., with the capacity to bind specifically to a (generally protein) factor important for gene expression, such that expression of the gene of interest, in this case Col 11A1, is inhibited, etc. Other illustrative, non-limiting examples of Col 11A1 inhibitors include compounds or substances capable of preventing Col 11A1 protein from performing its function or activity, for example, Col 11A1 inhibitor peptides, antibodies directed specifically against Col 11A1 epitopes, as well as non-peptide chemical compounds that reduce or inhibit Col 11A1 protein function.

Col 11A1 inhibitors of activators can be identified and evaluated according to the teachings of the present invention; particularly the previously described method of screening, can be used. Nevertheless, methods of another type suitable for identifying and evaluating Col 11A1 inhibitors or activators can be used.

Compounds causing the reduction or increase in levels of Col 11A1 mRNA can be identified using standard assays for determining mRNA expression levels, such as those mentioned in relation to the first method of the invention. Compounds causing the reduction or increase of the levels of Col 11A1 protein can be identified using standard assays for the determination of protein expression levels such as those mentioned in relation to the first method of the invention.

Illustrative, non-limiting examples of Col 11A1 inhibitors include:

a) specific antibodies against one or more epitopes present in the Col 11A1 protein (i.e., amino acid sequences encoded by exon 6A and/or exon 8), preferably human or humanized monoclonal antibodies, or functional fragments thereof, single-chain antibodies, anti-idiotype antibodies, etc.; in a particular embodiment, said antibody is 1E8.33 monoclonal antibody, a variant thereof, the characteristics of which are mentioned below;

b) cytotoxic agents, such as toxins, molecules with radioactive atoms, or chemotherapeutic agents, which include in a non-limiting manner small organic and inorganic molecules, peptides, phosphopeptides, antisense molecules, ribozymes, siRNAs, triple-helix molecules, etc., inhibiting Col 11A1 protein expression and/or activity; and

c) Col 11A1 protein antagonist compounds inhibiting one or more of said Col 11A1 protein functions (ie., regulation of osteoblast differentiation).

Methods of the invention have particular application in the healing of bone fractures. In cases where mineralization must be controlled such as in the healing of difficult bone fractures or in the case of large osteochondral defects that will not heal without intervention, Col 11A1 recombinant protein and the associated silencing antisense RNA or antisense morpholino oligonucleotides that decrease expression of Col 11A1 protein may be useful to optimize the mineralization and healing process.

Applications for bone fracture repair exist where enhancement of mineralization is beneficial. Biomaterials designed to promote or alternatively, inhibit mineralization may benefit from the inclusion of either the recombinant protein or the antisense morpholino oligonucleotides. For example, a biomaterial scaffold for the repair of cartilage or blood vessel should not mineralize, however, they often do. In contrast a scaffold to promote bone or tooth regeneration should mineralize.

### DEFINITIONS

The following definitions and introductory matters are applicable in the specification. The meaning of various terms and expressions as they are used within the context of the present invention is provided below to aid in understanding the present patent application.

By “bone mineralization disorder” is meant a disorder affecting mineralization of the bone matrix or any phenotype associated with the disorder. Matrix mineralization disorders and their associated phenotypes include, for example, rickets (defects in growth plate cartilage), osteomalacia, osteogenesis imperfecta, severe osteoporosis, and hypophosphatasia (HPP) (e.g., infantile HPP, childhood HPP, perinatal HPP, adult HPP, or odontohypophosphatasia), HPP-related seizure, premature loss of deciduous teeth, incomplete bone mineralization, elevated blood and/or urine levels of inorganic pyrophosphate (PPi), elevated blood and/or urine levels of phosphoethanolamine (PEA), elevated blood and/or urine levels of pyridoxal 5′-phosphate (PLP), inadequate weight gain, bone pain, calcium pyrophosphate dihydrate (CPPD) crystal deposition, and aplasia, hypoplasia or dysplasia of the dental cementum. Matrix mineralization disorders can be diagnosed, for example, by growth retardation with a decrease of long bone length (such as femur, tibia, humerus, radius, ulna), a decrease of the mean density of total bone and a decrease of bone mineralization in bones such as femur, tibia, ribs and metatarsi, and phalange, a decrease in teeth mineralization, and premature loss of deciduous teeth (e.g., aplasia, hypoplasia or dysplasia of dental cementum). Without being so limited, treatment of matrix mineralization disorders may be observed by one or more of the following: an increase of long bone length, an increase of mineralization in bone and/or teeth, a correction of bowing of the legs, a reduction of bone pain and a reduction of CPPD crystal deposition in joints.

By “pharmaceutical composition” is meant a composition containing a polypeptide or nucleic acid or other Col 11A1 modulator described herein, formulated with a pharmaceutically acceptable carrier/excipient as part of a therapeutic regimen for the treatment of disease/injury or bone development in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for subcutaneous administration; or any other formulation described herein.

By “pharmaceutically acceptable carrier” is meant an excipient or carrier that is physiologically acceptable to the treated subject while retaining the therapeutic properties of the compound with which it is administered. One exemplary pharmaceutically acceptable excipient is physiological saline. Other physiologically acceptable excipients and their formulations are known to one skilled in the art and described, for example, in “Remington: The Science and Practice of Pharmacy” (20th ed., ed. A. R. Gennaro, 2000, Lippincott Williams & Wilkins).

By “polypeptide” is meant any natural or synthetic chain of amino acids at least two amino acids in length, including those having post-translational modification (e.g., glycosylation or phosphorylation).

As used herein, when a polypeptide or nucleic acid sequence is referred to as having “at least X % sequence identity” to a reference sequence, it is meant that at least X percent of the amino acids or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith et al., J. Mol. Biol. 147:195-7, 1981) and BLAST (Basic Local Alignment Search Tool; Altschul et al. J. Mol. Biol. 215: 403-10, 1990). These and other alignment algorithms are accessible using publicly available computer software such as “Best Fit” (Smith and Waterman, Advances in Applied Mathematics, 482-489, 1981) as incorporated into GeneMatcher Plus™ (Schwarz and Dayhoff, Atlas of Protein Sequence and Structure, Dayhoff, M. O., Ed pp 353-358, 1979), BLAST, BLAST-2, BLAST-P, BLAST-N, BLAST-X, WU-BLAST-2, ALIGN, ALIGN-2, CLUSTAL, or Megalign (DNASTAR). In addition, those skilled in the art can determine appropriate parameters for measuring alignment, including any algorithms needed to achieve optimal alignment over the length of the sequences being compared. In general, for polypeptides, the length of comparison sequences can be at least five amino acids, preferably 10, 20, 30, 40, 50, 60, 70, 80, 90, 100, 125, 150, 175, 200, 250, 300, 400, 500, 600, 700, or more amino acids, up to the entire length of the polypeptide. For nucleic acids, the length of comparison sequences can generally be at least 10, 20, 30, 40, 50, 60, 70, 80, 90, 100, 125, 150, 175, 200, 250, 300, 400, 500, 600, 700, 800, 900, 1000, 1100, 1200, 1300, 1400, 1500, 1600, 1700, 1800, 1900, 2000, 2100, or more nucleotides, up to the entire length of the nucleic acid molecule. It is understood that for the purposes of determining sequence identity when comparing a DNA sequence to an RNA sequence, a thymine nucleotide is equivalent to a uracil nucleotide.

A homologous nucleotide sequence can further contain non-silent mutations, i.e. base substitutions, deletions, or additions resulting in amino acid differences in the encoded polyaminoacid, so long as the sequence remains at least about 70% identical to the polyaminoacid encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tyrptophan and phenylalanine.

Homologous nucleotide sequences can be determined by comparison of nucleotide sequences, for example by using BLAST N, above. Alternatively, homologous nucleotide sequences can be determined by hybridization under selected conditions. For example, the nucleotide sequence of a second polynucleotide molecule is homologous to SEQ ID NO:1 (or any other particular polynucleotide sequence) if it hybridizes to the complement of SEQ ID NO:1 under moderately stringent conditions, e.g., hybridization to filter-bound DNA in 0.5 M NaHPO₄, 7% sodium dodecyl sulfate (SDS), 1 mM EDTA at 65° C., and washing in 0.2×SSC/0.1% SDS at 42° C. (see Ausubel et al editors, Protocols in Molecular Biology, Wiley and Sons, 1994, pp. 6.0.3 to 6.4.10), or conditions which will otherwise result in hybridization of sequences that encode a Col 11A1 protein as defined below. Modifications in hybridization conditions can be empirically determined or precisely calculated based on the length and percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

In another embodiment, a second nucleotide sequence is homologous to SEQ ID NO:1 (or any other sequence of the invention) if it hybridizes to the complement of SEQ ID NO:1 under highly stringent conditions, e.g. hybridization to filter-bound DNA in 0.5 M NaHPO4, 7% SDS, 1 mM EDTA at 65° C., and washing in 0.1×SSC/0.1% SDS at 68° C., as is known in the art.

By “subject” is meant any mammal, e.g., a human.

By “therapeutically effective amount” is meant an amount of a nucleic acid or polypeptide of the invention that is sufficient to substantially treat, prevent, delay, suppress, or arrest any symptom of a condition to which bone mineralization is desired to be modulated. A therapeutically effective amount of a compound of the invention may depend on the severity of the matrix mineralization desired and the condition, weight and general state of the subject and can be determined by ordinarily-skilled artisan with consideration of such factors. A therapeutically effective amount of a compound of the invention can be administered to a mammal in a single dose or in multiple doses administered over a period of time.

By “treating” is meant administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease or undesirable condition to improve or stabilize the subject's condition. Thus, in the claims and embodiments, treating is the administration to a subject either for therapeutic or prophylactic purposes.

The term “antagonist” refers to any molecule that inhibits the biological activity of the molecule being agonized. Examples of antagonist molecules include, among others, proteins, peptides, natural peptide sequence variations and small organic molecules (having a molecular weight less than 500 daltons).

The term “antibody” refers to a glycoprotein that exhibits specific binding activity for a particular protein, which is referred to as “antigen”. The term “antibody” comprises whole monoclonal antibodies or polyclonal antibodies, or fragments thereof, and includes human antibodies, humanized antibodies and antibodies of a non-human origin. “Monoclonal antibodies” are homogenous, highly specific antibody populations directed against a single site or antigenic “determinant”. “Polyclonal antibodies” include heterogeneous antibody populations directed against different antigenic determinants.

As it is used herein, the term “epitope” refers to an antigenic determinant of a protein, which is the amino acid sequence of the protein which a specific antibody recognizes. The term “specificity” refers to the detection of false positives; 100% specificity means that there are no false positives.

The term “Col 11A1” gene refers to the gene encoding Human Col 11A1 (also known as COLL6 or STL2), the reference gene sequence of which is NP_542196.2 occupies about 150 kilobases (kb), contains 68 exons, is located in chromosome 1 (1p21) between the base pairs 103342023 and 103574052, and encodes a protein with 1818 amino acids (according to the isoform) and 181 KDa containing a signal peptide (1-36 amino acids). This gene is conserved in humans, chimpanzees, cows, chickens, mice, rats and zebra fish. As it is used herein, the term “Col 11A1” does not refer only to the human gene but also to the orthologs of other species.

The terms “individual” or “subject” refer to members of mammal species, and includes but is not limited to domestic animals, primates and humans; the subject is preferably a male or female human being of any age or race.

The term “oligonucleotide primer” or “primer” refers to a nucleotide sequence which is complementary to a nucleotide sequence of the Col 11A1 gene. Each primer hybridizes with its target nucleotide sequence and acts like a DNA polymerization initiation site.

The term “protein” refers to a molecular chain of amino acids with biological activity. The term includes all forms of post-translation modifications, for example glycolysation, phosphorylation or acetylation.

The term “probe” refers to a complementary nucleotide sequence of a nucleotide sequence derived from the Col 11A1 gene which can be used for detecting that nucleotide sequence derived from the Col 11A1 gene.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

The term “adjuvant” refers to a compound that enhances the effectiveness of the vaccine, and may be added to the formulation that includes the immunizing agent. Adjuvants provide enhanced immune response even after administration of only a single dose of the vaccine. Adjuvants may include, for example, muramyl dipeptides, pyridine, aluminum hydroxide, dimethyldioctadecyl ammonium bromide (DDA), oils, oil-in-water emulsions, saponins, cytokines, and other substances known in the art. Examples of suitable adjuvants are described in U.S. Patent Application Publication No. US2004/0213817 A1.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 (A-B) show MicroCT images of whole body for WT and Col 11A1-deficient littermates at e17.5d. FIG. 1A shows wild type (WT) mouse; FIG. 1B shows Col 11A1-deficient mouse. Differences between WT and the Col 11A1-deficient mice were consistent with previous characterization which focused on changes to the cartilage. These images are representative of all WT and mutant mice included in this study.

FIG. 2 show three-dimensional reconstructions from X-ray micro-CT data of axial skeleton and ribs; Differences in spinal curvature and length are apparent upon comparison, as well as a decrease in the separation between vertebrae in the mutant mouse. Lumbar vertebrae from the Col 11A1-deficient mouse were less mineralized than the WT mouse and were not visible by micro-CT. These images are representative of all WT and mutant mice included in this study.

FIG. 3 show pairwise comparison of shape and size of individual vertebrae. Left column shows cervical vertebrae C2 through C7. Middle column shows thoracic vertebrae T1 through T7. Right column show thoracic vertebrae T8 through T13. Differences in shape and surface characteristics are indicated. Vertebral bodies of T8-T11 were less mineralized in the Col 11A1-deficient mouse compared to WT. Vertebral bodies in WT mouse in comparison to Col 11A1-deficient mouse show evidence of hemivertebrae malformation with decreased mineralization along the midline of the vertebra.

FIG. 4 (A-F) show comparison of ribs between WT and Col 11A1-deficient mice. FIG. 4A, FIG. 4C, FIG. 4E show WT mice and FIG. 4B, FIG. 4D, FIG. 4F show Col 11A1-deficient mouse. FIG. 4A and FIG. 4B show histological differences in four adjacent ribs for WT and Col 11A1-deficient mice. Trichrome stain rendered mineralized tissue green, cartilage tissue deep blue, and blood cells pink/purple. Changes to the lower hypertrophic region and zone of mineralization are apparent. FIG. 4C and FIG. 4D show Col 11A1-deficiency led to an increase in mineralization immediately adjacent to the hypertrophic zone. FIG. 4E and FIG. 4F show reduced length, increased curvature of the ribs was apparent in the Col 11A1-deficient mice compared to WT. Proximal is oriented to the left for each rib, with the distal growth plate located on the right. Scale bars E and F=1.0 mm.

FIG. 5 (A-F) show histological differences in the humeri of WT and Col 11A1-deficient mice. FIG. 5A, FIG. 5C, FIG. 5E show WT mice and FIG. 5B, FIG. 5D, FIG. 5F show Col 11A1-deficient mice. Trichrome staining was used to identify mineralized tissue (green), compared to cartilage tissue (blue). FIG. 5A and FIG. 5B show upper and lower hypertrophic and mineralized zone. FIG. 5C and FIG. 5D show proliferative and hypertrophic zone of the growth plate demonstrating altered cellular density, cell size, and organization within the cartilage. FIG. 5E and FIG. 5F show the mineralized zone immediately adjacent to growth plate from WT and Col 11A1-deficient mice. Scale bar A and B=0.5 mm; scale bars C, D, E, and F=0.1 mm.

FIG. 6 (A-D) are X-ray micro-CT comparison of humerus and femur longitudinal cross-section of WT and Col 11A1-deficient mice. FIG. 6A shows WT humerus. FIG. 6B shows WT femur. FIG. 6C shows mutant humerus. FIG. 6D shows mutant femur. Thin sections were created from X-ray micro-CT reconstructions. Mineralized tissue was assigned a color dependent upon three density ranges: a low density range (green), intermediate density (blue) and high density (white). Scale bar=1 mm.

FIG. 7 (A-D) are X-ray micro-CT images of forelimbs. FIG. 7A and FIG. 7B show Col 11A1-deficient mouse. FIG. 7C and FIG. 7D show WT mouse; FIG. 7A shows the radius, ulna and humerus of Col 11A1-deficient mouse. Bones were shorter and wider in the Col 11A1-deficient mice compared to FIG. 7C. WT littermates. Deltoid tuberosity was apparent in the WT humerus but absent in the Col 11A1-deficient mouse. FIG. 7B and FIG. 7D show longitudinal cross-sections of each forelimb, shown in FIG. 7A and FIG. 7C, respectively. Mineralized tissue was assigned a color dependent upon three density ranges: low density range (green), intermediate density (blue) and high density (white). Marrow space within the Col 11A1-deficient limb showed regions of higher bone density near the proximal growth plates and those of very low density near the distal growth plates when compared to analogous regions in the WT littermate. Scale bars=0.5 mm.

FIG. 8 (A-B) show cross-section of humeri at diaphysis, distal and proximal metaphyses. FIG. 8A shows WT mouse and FIG. 8B shows Col 11A1-deficient mouse. Col 11A1-deficient humerus was wider and more cylindrical than WT. Mineralized tissue was assigned a color dependent upon three density ranges: low density range (green), intermediate density (blue) and high density (white). Trabecular bone was more dense in Col 11A1-deficient mice compared to WT at proximal metaphysis. Trabecular bone is less dense in Col 11A1-deficient mice compared to WT at distal metaphysis. Bone collar is less dense but thicker in Col 11A1-deficient mice compared to WT littermate. Scale bars=0.5 mm.

FIG. 9 shows endochondral ossification. Endochondral ossification takes place within the growth plate and involves the differentiation of chondrocytes through resting, proliferative, prehypertrophic, and hypertrophic stages. In the adjacent tissues, preosteoblasts respond to local cues from nearby tissues to differentiate along the osteoblast lineage. New bone formation occurs adjacent to the hypertrophic and prehypertrophic zones as cells from the periosteum form the bone collar and newly differentiated osteoblasts from the primary trabeculae of the mineralized zone. PTHrP and BMP-2 are key players in the process of endochondral ossification. While PTHrP is known for its role in maintaining chondrocytes proliferation and inhibiting terminal differentiation, BMP-2 is an inducer of both chondrocyte hypertrophy and osteoblast differentiation via the canonical SMAD signaling pathway. Col 11A1 is an extracellular matrix molecule that undergoes alternative splicing to give rise to distinct splice forms that vary with respect to the inclusion and exclusion of exons 6A, 6B, 7, and 8. Splice forms that include exons 6A, 7, and 8 are synthesized by osteoblasts and may promote osteoblast differentiation, while splice forms that include exon 6B are synthesized only by prehypertrophic chondrocytes located within a restricted zone immediately adjacent to the perichondrium/periosteum and may form a boundary between cartilage and newly forming bone collar.

FIG. 10 shows the amino terminal domain of Col 11A1 undergoes alternative splicing that can result in several different splice forms. Four exons, 6A, 6B, 7, and 8, encode the variable region positioned between the amino propeptide (Npp), encoded by exons 2-5, and the minor triple helix. Each splice variant is expressed in a distinct spatial and temporal manner during endochondral ossification. The exact function of Col 11A1 alternative splicing is yet to be determined.

FIG. 11 (A-D) show longitudinal section of representative humeri and femurs. FIG. 11A and FIG. 11B show WT mouse and FIG. 11C and FIG. 11D show Col 11A1-deficient mouse. FIG. 11A and FIG. 11C are humeri; FIG. 11B and FIG. 11D are femurs. Mineralized tissue was analyzed by X-ray microCT. Density ranges are depicted by color: low density range (green), intermediate density (blue) and high density (white). Abnormalities are apparent in both trabecular bone and bone collar. Scale bar=1 mm.

FIG. 12 (A-H) show osteoblast differentiation marker expression compared to the minor fibrillary collagen XI alpha 1. Total RNA was isolated from pluripotent mesenchymal C2C12 cells treated with BMP-2 (300 ng/mL) for the indicated number of days. Relative expression is reported as 2^{-ACT}. All samples were normalized to housekeeping gene peptidylprolyl isomerase A (PPIA). FIG. 12A and FIG. 12B show day 6 change in morphology from mesenchymal spindle-shaped cells to osteoblastic cuboidal shape was observed. FIG. 12C, FIG. 12D, FIG. 12E, and FIG. 12F show day 2 expression levels of ALP, Runx2, and Col 1a1 were markedly increased in BMP-2-treated samples compared to control. ALP mRNA levels decreased after day 2 and Runx2 and Col 1a1 levels dropped after day 3. OCN mRNA levels increased persistently up to day 6. FIG. 12G shows expression levels of Col5a1 mRNA increased throughout the six-day experiment. FIG. 12H shows overall expression levels of Col 11A1 mRNA increased to day 2 and then decreased gradually on days 3 and 6. Results represent mean±SEM n=3 in each group. Scale bar=200 μm.

FIG. 13 (A-D) show BMP-2 regulates Col 11A1 mRNA levels and alternative splicing in a time-dependent manner. Total RNA was isolated from pluripotent mesenchymal C2C12 cells treated with BMP-2 (300 ng/mL) for the indicated number of days. Relative expression is reported as 2^{-ACT}. All samples were normalized to housekeeping gene peptidylprolyl isomerase A (PPIA). Upregulated expression of exon 6A peaked on day 3 and remained high up to day 6; whereas, exons 7 and 8 mRNA levels peaked on day 2 and remained upregulated until day 6. In contrast, BMP-2 induced a spike in exon 6B expression on day 3 that was followed by a decrease. Results represent mean±SEM n=3 in each group. FIG. 13A shows e6A, FIG. 13B shows e6B, FIG. 13C shows e7, FIG. 13D shows e8,

FIG. 14 (A-B) show expression of Col 11A1 was reduced significantly when SMAD 4 expression is knocked down. Cells were treated with siRNA targeting SMAD 4. FIG. 14A shows mRNA levels for SMAD 4 were quantified by real time PCR to assess the effectiveness of the technique. FIG. 14B shows expression of Col 11A1 was induced by treatment with BMP-2, and this effect was blocked by treatment with SMAD 4 siRNA. The splice form of Col 11A1 containing exons 6A-7-8 was reduced significantly in the absence of SMAD 4 compared to control C2C12 cells. Statistical significance was calculated using one-way ANOVA with Bonferroni's Multiple Comparison post hoc test. Results represent mean±SEM n=3 in each group. * indicates p<0.05, ** indicates p<0.01, *** indicates p<0.001, and **** indicates p<0.0001, and n.s. is not significant.

FIG. 15 (A-B) show PTHrP alters BMP-2-induced expression of Col 11A1 alternative exons. Pluripotent mesenchymal C2C12 cells were treated with BMP-2 (300 ng/mL) for five days and PTHrP (10⁻⁷ M) was added to specific samples for 24 h. As control, cells were incubated in the absence of growth factors or presence of PTHrP only. Col 11A1 exon expression was assessed by FIG. 15A semiquantitative and FIG. 15B quantitative real-time PCR using different sets of primers. Relative expression is reported as 2^{-ACT}. All samples were normalized to housekeeping gene peptidylprolyl isomerase A (PPIA). FIG. 15A and FIG. 15B show PTHrP alone did not induce any significant changes in Col 11A1 exon expression. In contrast, combined with BMP-2, PTHrP reduced BMP-2-stimulated expression of exons 6A and 7. Further, PTHrP was able to increase exon 6B expression with BMP-2, although this effect was statistically not significant. Statistical significance was calculated using two-way ANOVA with Bonferroni's Multiple Comparison post hoc test. Results represent mean±SEM n=3 in each group. * indicates p<0.05, ** indicates p<0.01, *** indicates p<0.001, and **** indicates p<0.0001.

FIG. 16 (A-F) show the effects of Col 11A1 knockdown on the expression of ALP, OCN, Runx2, and Col 1a1 is time-dependent. FIG. 16A and FIG. 16B show pluripotent C2C12 cells were transfected with either Col 11A1 or scramble siRNA. Cells were stimulated with BMP-2 (300 ng/mL) for 24 h and 72 h. Relative expression is reported as 2^{-ACT}. All samples were normalized to housekeeping gene peptidylprolyl isomerase A (PPIA). FIG. 16C show at 24 h BMP-2 stimulation, ALP expression was significantly decreased in Col 11A1 deficient cells as compared to control. In contrast, at 72 h BMP-2 stimulation, Col 11A1 deficient cells showed a marked increase in ALP expression as compared to control. FIG. 16D shows Runx2 mRNA levels were not significantly affected by Col 11A1 knockdown at 24 h of BMP-2 stimulation, however at 72 h, Runx2 mRNA levels were significantly higher in Col 11A1 deficient cells as compared to control. FIG. 16E shows OCN mRNA levels were not significantly different in Col 11A1-deficient cells as compared to control at 24 h BMP-2 treatment. By 72 h however, Col 11A1-deficient cells expressed lower levels of OCN as compared to control. FIG. 16F shows Col 1a1 mRNA levels were not significantly affected by Col 11A1 knockdown at 24 h BMP-2 stimulation; however at 72 h Col 1a1 mRNA levels were significantly higher in Col 11A1 deficient cells as compared to control. Statistical significance was calculated using two-way ANOVA with Bonferroni's Multiple Comparison post hoc test. Results are reported as the mean±SEM n=3 in each group. * indicates p<0.05, ** indicates p<0.01, *** indicates p<0.001, and **** indicates p<0.0001, and n.s. is not significant.

FIG. 17 (A-I) show Col 11A1 knockdown negatively affects canonical BMP-2 induced phosphorylation and nuclear localization of SMAD. C2C12 cells were plated on glass coverslips at 2×10⁴ cells/cm². Cells were transfected with either Col 11A1 or scramble siRNA for 24 h and then stimulated with BMP-2 for 30 minutes. Antibodies against phospho-SMAD1/5/8 were used to assess the effects of Col 11A1 knockdown on BMP-2 mediated SMAD1/5/8 phosphorylation. FIG. 17A, FIG. 17D, FIG. 17G show DAPI staining was used to identify the nuclei of cells. FIG. 17B, FIG. 17E, FIG. 17H show the antibody to phosphorylated SMAD identified background levels in the absence of BMP-2 FIG. 17B, an increased level upon treatment with BMP-2 as well as nuclear localization FIG. 17E, and a perinuclear staining pattern for phosphorylated SMAD under conditions of reduced Col 11A1 expression FIG. 17H. Overlay of DAPI and p-SMAD is shown in FIG. 17C, FIG. 17F, and FIG. 17I. No phospho-SMAD1/5/8 was detected in the nuclei of Col 11A1 deficient cells as compared to control.

FIG. 18 (A-B) show the quantification of SMAD1 and phospho-SMAD1/5/8 levels. FIG. 18A shows western blot analysis was used to measure the total SMAD1 level and phosphorylated SMAD 1/5/8 levels in Col 11a1-deficient C2C12 cells compared to controls. FIG. 18B shows a 40% decrease in phospho-SMAD1/5/8 levels was detected compared to control. In contrast, SMAD1 levels were 27% higher in Col 11A1 deficient cells as compared to control. Results represent mean±SEM n=3 in each group.

FIG. 19 (A-I) show Col 11A1 knockdown negatively affects canonical BMP-2 induced phosphorylation and nuclear localization of SMAD. Cells were plated and treated as described in FIG. 17. Antibodies against total SMAD 1 were used to assess the effects of Col 11A1 knockdown on BMP-2 mediated SMAD 1 localization to the nucleus. FIG. 19A, FIG. 19D, FIG. 19G show DAPI staining was used to identify the nuclei of cells. FIG. 19B, FIG. 19E, FIG. 19H show the antibody to total SMAD 1 identified pre-induction levels in the absence of BMP-2, FIG. 19B, an increased level upon treatment with BMP-2 as well as nuclear localization, FIG. 19E, and a perinuclear staining pattern for total SMAD 1 under conditions of reduced Col 11A1 expression, FIG. 19H. Overlay of DAPI and SMAD 1 is shown in FIG. 19C, FIG. 19F, and FIG. 19I. No SMAD 1 was detected in the nuclei of Col 11A1 deficient cells as compared to control.

FIG. 20 (A-D) show BMP-2-induced expression of ALP, OCN, Runx2, and Col1a1 is altered by recombinant Col 11A1 NTD fragments in a spliceform-specific manner. FIG. 20A shows BMP-2 unregulated ALP expression was significantly reduced upon incubation with recombinant Col 11A1 [p6B-7] and Col 11A1[p7-8] NTD fragments. FIG. 20B and FIG. 20C show OCN and Runx2 mRNA levels were markedly increased when recombinant Col 11A1 [p6B-7] and Col 11A1 [p7-8] NTD fragments were added to BMP-2 treated cells as compared to control. FIG. 20D shows Col 1a1 expression was enhanced by addition of recombinant Col 11A1 [p7-8] NTD fragment but reduced by recombinant Col 11a1 [p6B-7] NTD. Statistical significance was calculated using student's paired t-test. Results represent mean±SEM n=3 in each group. * indicates p<0.05, ** indicates p<0.01, *** indicates p<0.001, and **** indicates p<0.0001, and n.s. is not significant.

FIG. 21 shows recombinant Col 11A1[p6B-7] NTD fragment but not Col 11A1 [p7] NTD fragment reduces BMP-dependent luciferase activation in C2C12 cells. C2C12 cells were transfected with a BMP-responsive firefly luciferase reporter plasmid and a control pCMV-β-gal reporter plasmid. BMP-2 (300 ng/mL) and/or recombinant Col 11A1[p6B-7] NTD fragment and recombinant Col 11A1 [p7] NTD fragment were added to cultures. Cell lysates were analyzed for luciferase activity. Relative luciferase activity was calculated as the ratio of luciferase to β-galactosidase activity, to control for transfection efficiency, and is expressed as a multiple of the activity of unstimulated cells transfected with reporter alone (control). Recombinant Col 11A1 [p6B-7] NTD fragment significantly reduced BMP-2-induced relative luciferase activity. Similarly, Col 11A1 knockdown diminished BMP-2-induced luciferase activity. In contrast, recombinant Col 11A1[p7] NTD fragment neither enhanced nor reduced BMP-2-induced luciferase activity. Statistical significance was calculated using student's paired t-test. Results represent mean±SEM n=3 in each group. * indicates p<0.05, ** indicates p<0.01, *** indicates p<0.001, and **** indicates p<0.0001, and n.s. is not significant.

### DETAILED DESCRIPTION OF THE PREFERRED EMBODIMENTS

Bone is a composite matrix composed of mineralized and aligned collagen nanofibers. A combination of inorganic apatite nanocrystals and organic collagen fibers provides bone with unique mechanical and biological properties. The apatite nanocrystals provide osteoconductivity and compressive strength while the collagen fibers provide elasticity and a template for mineralization and maturation of osteoprogenitor cells. Unique factors that contribute to bone toughness are the aligned network of collagen fibers, apatite nanocrystals, and proteins in the bone extracellular matrix (ECM) that link the apatite crystals to the collagen fibers. On a larger scale, laminated multilayers of calcium phosphate (CaP)-deposited aligned fibers form the cortical bone that is composed of osteons having microtube-like structures surrounding a central micro-canal that provides nutrient/waste transport to and from the bone tissue.

Collagen is the main component of the extracellular matrix (ECM). The correct expression of the genes encoding the different types of collagen is necessary for the correct assembly of the ECM during embryonic development and for maintenance thereof in the adult organism. Collagen XI (COL11) is a type of collagen that has been studied very little but plays a fundamental role in regulating fibril networks in cartilaginous and non-cartilaginous matrices (Li, Y., et al., Cell, 1995, 80:423-430); these fiber networks are involved in different morphogenesis processes during embryonic development in vertebrates. Transcripts of collagen XI alpha 1 chain (Col 11A1) have been found during fetal development fetal in cartilaginous tissues and also in other tissues such as bone, kidney, skin, muscle, tongue, intestine, liver, ear, brain and lung (Sandberg, J. M., et al., Biochem. J., 1993, 294:595-602; Yoshioka, H., et al., Dev. Dyn., 1995, 204: 41-47). The ECM also plays an important role in certain biological processes, such as cell differentiation, proliferation and migration; therefore, the dysregulation of the expression of genes encoding the proteins making them up is associated with carcinogenic and metastatic processes (Boudreau, N., and Bissell, M. J., Curr. Opin. Cell Biol., 1998, 10:640-646; Stracke, M. L., et al., In vivo, 1994, 8:49-58). In the particular case of Col 11A1, stroma fibroblasts have been proven to have high Col 11A1 gene expression levels in sporadic colorectal carcinomas, whereas this gene is not expressed in healthy colon (Fischer, H., et al., Carcinogenesis, 2001, 22:875-878). Col 11A1 gene expression has also been associated with pancreatic, breast, colon, lung, head and neck cancer (Kim, H. et al., BMC Medical Genomics, 2010, 3:51; lacobuzio-Donahue, C., Am. J. Pathology, 2002, 160(4):1239-1249; Ellsworth, R. E., et al., Clin. Exp. Metastasis, 2009, 26: 205-13; Feng, Y., et al., Breast Cancer Res. Treat., 2007, 103(3):319-329; J.Gast.Liv.dis., 2008; Fischer, H., et al., BMC Cancer, 2001, 1:17-18; Fischer, H., et al., Carcinogenesis, 2001, 22:875-878; Suceveanu, A. I., et al., J. Gastrointestin. Liver Dis, 2009, 18(1):33-38; Chong, I W, et al., Oncol Rep, 2006, 16(5):981-988; Whan, K., Oncogene, 2002, 21:7598-7604; OncolRep, 2007; Schmalbach., C. E., et al., Arch. Otolaryngol. Head Neck Surg., 2004, 130(3):295-302) and bladder cancer (WO 2005/011619), and Col 11A1 protein expression has been associated with pancreatic and colon cancer (Pilarsky, C., et al., J. Cel. Mol. Med., 2008, 12(6B):2823-35; Erkan, M., et al., Mol. Cancer, 2010, 9:88-103; Bowen, K. B., et al., J. Hist. Cyt., 2008, 56(3):275-283).

Applicants have found that the Col 11A1 gene plays a role in bone mineralization and modulation of the same can provide increased or decrease bone mineralization as desired. In a non-limiting example, NTD fragments of Col 11A1 comprising exon 8 stimulate bone mineralizing properties. In contrast, NTD fragments of Col 11A1 lacking exon 8 and/or comprising exon 6B inhibit bone mineralizing properties.

### Col 11A1

The Col 11A1 gene encodes a protein with 1818 amino acids, whose triple-helical region is between amino acids 529 and 1542. It has two domains that are not always present in the mature protein, the C-terminal domain (amino acids 1564-1806) and the N-terminal domain (amino acids 37-511). It forms part of collagen type XI, which is formed in cartilage by three chains forming a triple helix, α1(XI), α2(XI) (encoded by the COL11A2 gene) and α3(XI) (generated by excessive glycosylation of α1(II)) which can be substituted with α1(V). It is a component of the hyaline cartilage ECM, although it is also expressed in non-cartilaginous tissues and in tumor or virus-transformed cell lines, but in this case the three chains of collagen type XI are not always co-expressed, which could mean that the fibers have a chain composition different from that of cartilage, being homotrimeric or heterotypic in these locations (Yoshioka, H., J Biol Chem, 1990; 265(11):6423-6426; Lui, LCH, Biochem J, 1995; 311:511-516). It is thought to participate in the fibrillogenesis, regulating lateral growth of collagen II fibers, serving as a support for said fibers and being located within the formed fiber (Weis, M A., J Biol Chem 2010; 285(4):2580-2590). It is synthesized like procollagen, which is proteolytically processed after secretion, terminal N peptide (37-511) and terminal C peptide (1564-1806) being removed (Halsted, K C., Mod Pathol 2008; 21(10):1246-1254). A TSP (38-229) or Npp (amino propeptide) region, which is also found in 7 other types of collagens, in laminin and in thrombospondin, is contained in the peptide amino terminal (NTD), and it contains a BMP-1 processing site (Warner, L., J Biol Chem 2006; 281(51):39507-39515, Gregory, K E., J Biol Chem 2000; 275(15): 11498-11506). In the case of α1 (XI), this region is not always removed, sometimes being exposed on the surface of the collagen fibers for a long time (Fallahi, A., Prot Sci 2005; 14:1526-1537). This region is very similar to the LNS domains, having potential binding sites for heparin and calcium, which could mean cell-ECM communication activity by binding to heparan sulfate proteoglycans (Warner, L., J Biol Chem 2006; 281(51):39507-39515, Fallahi, A., Prot Sci 2005; 14:1526-1537), even after being proteolyzed from the helical domain. Furthermore, the NTD covers a variable region which has different sequences and characteristics according to alternative splicing, combining exons 6-7-8 of the gene. These variants present tissue and temporal specificity (Warner, L., J Biol Chem 2006; 281(51):39507-39515) and at the same time affect Npp processing time.

According to the invention, Applicants have discovered that Col 11A1 plays an important role in bone mineralization. The present invention relates to the discovery that the collagen 11a1 (Col 11A1) protein, and/or fragments thereof, may be used to modulate bone mineralization. In some embodiments, bone mineralization is promoted by the addition of Col 11A1 or a fragment thereof, by pharmaceutical compositions that increase the presence of Col 11A1, and in some embodiments, bone mineralization may be inhibited by pharmaceutical compositions that interfere, impede, or inhibit Col 11A1.

Accordingly, the invention includes compositions including a Col 11A1 polypeptide, or fragment and compositions including a nucleic acid that encodes a Col 11A1 polypeptide or fragment. The invention also provides methods and kits for using such polypeptides and nucleic acids to treat bone mineralization disorders, and promote bone growth and wound healing.

Accordingly, the invention includes compositions including a Col 11A1 polypeptide, or fragment and compositions including a nucleic acid that encode a Col 11A1 polypeptide or fragment. The invention also provides methods and kits for using such polypeptides and nucleic acids to treat bone mineralization disorders, and promote bone growth and wound healing.

### Col 11A1 Polypeptides

The present disclosure provides Col 11A1 polypeptides, variants and fragments there of capable of stimulating bone mineralization. In one embodiment, the Col 11A1 is a Col 11A1 fragment of SEQ ID NO:1 or a sequence with 90% or greater identity thereto that is capable of stimulating bone mineralization. In an exemplary embodiment the Col 11A1 polypeptides, variants and fragments there of capable of stimulating bone mineralization comprise exon 6A and/or exon 8.

A variant Col 11A1 polypeptide can comprise an amino acid sequence having at least about 85%, at least about 90%, at least about 95%, at least about 98%, up to about 99%, amino acid sequence identity to the amino acid sequence set forth in SEQ ID NO:1, 2, or 3 and is capable of stimulating bone mineralization as determined by the methods and assays disclosed hereinafter and in the following examples.

In some embodiments, a Col 11A1 polypeptide comprises one or more modifications such as: 1) a poly(ethylene glycol) (PEG) moiety; 2) a saccharide moiety; 3) a carbohydrate moiety; 4) a myristyl group; 5) a lipid moiety; and the like.

In some embodiments, the Col 11a1 polypeptide comprises a protein transduction domain. “Protein Transduction Domain” or PTD refers to a polypeptide, polynucleotide, carbohydrate, or organic or inorganic compound that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of the polypeptide. In some embodiments, a PTD is covalently linked to the carboxyl terminus of the polypeptide.

A Col 11A1 polypeptide will in some embodiments be linked to (e.g., covalently or non-covalently linked) a fusion partner, e.g., a ligand; an epitope tag; a peptide; a protein other than the Col 11A1 polypeptide; and the like. Suitable fusion partners include peptides and polypeptides that confer enhanced stability in vivo (e.g., enhanced serum half-life); provide ease of purification, e.g., (His)ₙ, e.g., 6His, and the like; provide for secretion of the fusion protein from a cell; provide an epitope tag, e.g., GST, hemagglutinin, and the like; provide a detectable signal, e.g., an enzyme that generates a detectable product (e.g., β-galactosidase, luciferase), or a protein that is itself detectable, e.g., a green fluorescent protein, a red fluorescent protein, a yellow fluorescent protein, etc.; provides for multimerization, e.g., a multimerization domain such as an Fc portion of an immunoglobulin; and the like.

The Col 11A1 polypeptide can be made using any of a variety of established methods, e.g., conventional synthetic methods for protein synthesis; recombinant DNA methods; etc.

The present disclosure provides a composition comprising a Col 11A1 polypeptide. The composition can comprise, in addition to the Col 11A1 polypeptide, one or more of: a salt, e.g., NaCl, MgCl₂, KCl, MgSO₄, etc.; a buffering agent, e.g., a Tris buffer, N-(2-Hydroxyethyl) piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino) propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

### Nucleic Acids

The present disclosure provides a nucleic acid comprising a nucleotide sequence encoding a Col 11A1 polypeptide or fragment thereof. In some embodiments, the nucleic acid is an expression vector that, when introduced into a host cell, provides for production of a Col 11A1 polypeptide or fragment thereof. A nucleotide sequence encoding a Col 11A1 polypeptide or fragment thereof can be operably linked to one or more regulatory elements, such as a promoter and enhancer, that allow expression of the nucleotide sequence in the intended target cells (e.g., a cell that is genetically modified to synthesize the encoded Col 11A1 or fragment polypeptide).

Suitable promoter and enhancer elements are known in the art. For expression in a bacterial cell, suitable promoters include, but are not limited to, lad, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Large numbers of suitable vectors and promoters are known to those of skill in the art; many are commercially available for generating a subject recombinant construct. The following vectors are provided by way of example. Bacterial: pBs, phagescript, PsiX174, pBluescript SK, pBs KS, pNH8a, pNH16a, pNH18a, pNH46a (Stratagene, La Jolla, Calif, USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding a protein of interest. Suitable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

### Cells

The present disclosure provides isolated genetically modified host cells (e.g., in vitro cells) that are genetically modified with a subject nucleic acid. In some embodiments, a subject isolated genetically modified host cell can produce a Col 11A1 polypeptide or fragment thereof.

Suitable host cells include eukaryotic host cells, such as a mammalian cell, an insect host cell, a yeast cell; and prokaryotic cells, such as a bacterial cell. Introduction of a subject nucleic acid into the host cell can be effected, for example by calcium phosphate precipitation, DEAE dextran mediated transfection, liposome-mediated transfection, electroporation, or other known method.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Suitable yeast cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like.

Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed in the present invention include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some embodiments, the host cell is Escherichia coli.

### Pharmaceutical Compositions

The present disclosure provides pharmaceutical compositions comprising a Col 11A1 polypeptide or fragment thereof. In some embodiments, a subject composition comprises a Col 11A1 polypeptide or fragment thereof and a pharmaceutically acceptable carrier. The pharmaceutical composition can be administered to a host using any convenient means capable of resulting in the desired therapeutic effect. Thus, a Col 11A1 polypeptide or fragment thereof can be incorporated into a variety of formulations for therapeutic administration. More particularly, a Col 11A1 polypeptide or fragment thereof can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, and injections.

In pharmaceutical dosage forms, a Col 11A1 polypeptide or fragment thereof can be formulated alone or in appropriate association, as well as in combination, with other pharmaceutically active compounds. The following methods and excipients are merely exemplary and are in no way limiting.

For oral preparations, a Col 11A1 polypeptide or fragment thereof can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The pharmaceutical composition comprising a Col 11A1 polypeptide or fragment can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions comprising a Col 11A1 polypeptide or fragment thereof are prepared by mixing the polypeptide having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-Methylglucosamine, galactosamine, and neuraminic acid; and/or non-ionic surfactants such as Tween, Brij Pluronics, Triton-X or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration; see also Chen (1992) Drug Dev Ind Pharm 18, 1311-54.

Exemplary Col 11A1 concentrations in a subject pharmaceutical composition may range from about 1 mg/mL to about 200 mg/ml or from about 50 mg/mL to about 200 mg/mL, or from about 150 mg/mL to about 200 mg/mL.

An aqueous formulation of Col 11A1 polypeptide or fragment may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the Col 11A1 polypeptide or fragment formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 nM.

A surfactant may also be added to the Col 11A1 or fragment polypeptide formulation to reduce aggregation of the formulated polypeptide and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic™ F68 or Poloxamer 188™. Examples of suitable Polyoxyethylene alkyl ethers are those sold under the trademark Brij™. Exemplary concentrations of surfactant may range from about 0.001% to about 1% w/v.

A lyoprotectant may also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a subject formulation includes a Col 11A1 polypeptide or fragment thereof, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

Furthermore, a Col 11A1 polypeptide or fragment thereof can be made into suppositories by mixing with a variety of bases such as emulsifying bases or water-soluble bases. A Col 11A1 polypeptide or fragment thereof can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise a Col 11A1 polypeptide or fragment thereof in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of a Col 11A1 polypeptide or fragment thereof calculated in an amount sufficient to produce the desired effect in association with a pharmaceutically acceptable diluent, carrier or vehicle. The specifications for a Col 11A1 polypeptide or fragment thereof may depend on the particular polypeptide employed and the effect to be achieved, and the pharmacodynamics associated with each polypeptide in the host.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of polypeptide adequate to achieve the desired state in the subject being treated.

In some embodiments, a Col 11A1 polypeptide or fragment thereof is formulated in a controlled release formulation. Sustained-release preparations may be prepared using methods well known in the art. Suitable examples of sustained-release preparations include semipermeable matrices of solid hydrophobic polymers containing Col 11A1 polypeptide or fragment thereof in which the matrices are in the form of shaped articles, e.g. films or microcapsules. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(−)-3-hydroxybutyric acid. Possible loss of biological activity may be prevented or reduced by using appropriate additives, by controlling moisture content and by developing specific polymer matrix compositions.

A subject composition can include the Col 11A1 polypeptide or fragment thereof and may also include known antioxidants, buffering agents, and other agents such as coloring agents, flavorings, vitamins or minerals. For example, a subject formulation may also contain one or more of the following minerals: calcium citrate (15-350 mg); potassium gluconate (5-150 mg); magnesium citrate (5-15 mg); and chromium picollinate (5-200 μg). In addition, a variety of salts may be utilized, including calcium citrate, potassium gluconate, magnesium citrate and chromium picollinate. Thickening agents may be added to the compositions such as polyvinylpyrrolidone, polyethylene glycol or carboxymethylcellulose. Exemplary additional components of a subject formulation include assorted colorings or flavorings, vitamins, fiber, milk, fruit juices, enzymes and other nutrients. Exemplary sources of fiber include any of a variety of sources of fiber including, but not limited to: psyllium, rice bran, oat bran, corn bran, wheat bran, fruit fiber and the like. Dietary or supplementary enzymes such as lactase, amylase, glucanase, catalase, and the like can also be included. Chemicals used in the present compositions can be obtained from a variety of commercial sources, including, e.g., Spectrum Quality Products, Inc. (Gardena, Calif.), Sigma Chemicals (St. Louis, Mo.), Seltzer Chemicals, Inc., (Carlsbad, Calif.) and Jarchem Industries, Inc., (Newark, N.J.).

A subject formulation may also include a variety of carriers and/or binders. An exemplary carrier is micro-crystalline cellulose (MCC) added in an amount sufficient to complete dosage total weight. Carriers can be solid-based dry materials for formulations in tablet, capsule or powdered form, and can be liquid or gel-based materials for formulations in liquid or gel forms, which forms depend, in part, upon the routes of administration.

Exemplary carriers for dry formulations include, but are not limited to: trehalose, malto-dextrin, rice flour, micro-crystalline cellulose (MCC) magnesium sterate, inositol, fructo-oligosaccharide (FOS), gluco-oligosaccharide (GOS), dextrose, sucrose, and like carriers. Where the composition is dry and includes evaporated oils that produce a tendency for the composition to cake (adherence of the component spores, salts, powders and oils), dry fillers which distribute the components and prevent caking are included. Exemplary anti-caking agents include MCC, talc, diatomaceous earth, amorphous silica and the like, and are typically added in an amount of from approximately 1% to 95% by weight. It should also be noted that dry formulations which are subsequently rehydrated (e.g., liquid formula) or given in the dry state (e.g., chewable wafers, pellets, capsules, or tablets) can be used instead of initially hydrated formulations. Dry formulations (e.g., powders) may be added to supplement commercially available foods (e.g., liquid formulas, strained foods, or drinking water supplies). Similarly, the specific type of formulation depends upon the route of administration.

Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like).

Generally, water-based carriers possess a neutral pH value (e.g., pH 7.0+/−1.0 or 0.5 pH units). The compositions may also include natural or synthetic flavorings and food-quality coloring agents, all of which must be compatible with maintaining viability of the lactic acid-producing microorganism. Well-known thickening agents may also be added to the compositions such as corn starch, guar gum, xanthan gum, and the like.

A Col 11A1 polypeptide or fragment thereof can be formulated to be suitable for oral administration in a variety of ways, for example in a liquid, a powdered food supplement, a paste, a gel, a solid food, a packaged food, a wafer, a tablet, a lozenge, a capsule, and the like. Other formulations will be readily apparent to one skilled in the art.

### Methods of Increasing Bone Mineralization

The present disclosure provides methods of increasing bone mineralization. In some embodiments, the methods involve contacting a cell, tissue, or organ (in vitro, in vivo, or ex vivo) with an effective amount of a Col 11A1 polypeptide or fragment thereof.

An effective amount of a Col 11A1 polypeptide or fragment thereof is an amount that increases or reduces the level of Col 11a1 activity in a cell, tissue, or organ by at least about 10%, at least about 20%, at least about 25%, at least about 30%, at least about 40%, at least about 50%, at least about 60%, at least about 70%, at least about 80%, or more than 80%, compared to the level of Col 11A1 activity in the cell, tissue, or organ in the absence of a Col 11A1 polypeptide or fragment thereof.

An individual in need of a subject treatment method includes an individual in need of modulation of bone mineralization such as, for example after bone fracture, or perhaps after bone re-grating scaffolds have been put in place.

In some embodiments, the cells are in vitro. For example, the cells can be tissue culture cells. Alternatively, the cells can be obtained from a mammal.

Where a subject method involves administering an effective amount of a Col 11A1 polypeptide or fragment thereof to an individual, the polypeptide is administered to an individual using any available method and route suitable for drug delivery, including in vivo and ex vivo methods, as well as systemic and localized routes of administration. Conventional and pharmaceutically acceptable routes of administration include intranasal, intramuscular, intratracheal, subcutaneous, intradermal, topical application, intravenous, intraarterial, rectal, nasal, oral, and other enteral and parenteral routes of administration. Routes of administration may be combined, if desired, or adjusted depending upon the polypeptide and/or the desired effect. A polypeptide composition can be administered in a single dose or in multiple doses. In some embodiments, a Col 11A1 polypeptide or fragment thereof composition is administered orally. In some embodiments, a Col 11A1 polypeptide or fragment thereof composition is administered topically to the skin. In some embodiments, a Col 11A1 polypeptide or fragment thereof composition is administered locally. In some embodiments, a Col 11A1 polypeptide or fragment thereof composition is administered systemically.

### Screening Methods

The present disclosure also provides methods of identifying agents that increase or decrease the bone mineralizing properties (i.e, regulation of osteoblast diferentiaton) of a Col 11A1 polypeptide or fragment thereof. The methods generally involve contacting a Col 11A1 polypeptide or fragment with a test agent in the presence of a substrate for the Col 11A1 polypeptide or fragment; and determining the effect, if any, of the test agent on the activity of the Col 11A1 polypeptide or fragment. The method can be carried out in vitro in a cell-based assay system. Thus, the present disclosure provides an in vitro method for identifying an agent that increases or decreases the bone mineralizing properties of Col 11A1 polypeptide or fragment. As used herein, the term “determining” refers to both quantitative and qualitative determinations and as such, the term “determining” is used interchangeably herein with “assaying,” “measuring,” and the like. The terms “candidate agent,” “test agent,” “agent,” “substance,” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 and less than about 10,000 daltons, e.g., a candidate agent may have a molecular weight of from about 50 daltons to about 100 daltons, from about 100 daltons to about 150 daltons, from about 150 daltons to about 200 daltons, from about 200 daltons to about 500 daltons, from about 500 daltons to about 1000 daltons, from about 1,000 daltons to about 2500 daltons, from about 2500 daltons to about 5000 daltons, from about 5000 daltons to about 7500 daltons, or from about 7500 daltons to about 10,000 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Assays of the invention include controls, where suitable controls include a sample (e.g., a sample comprising the Col 11A1 polypeptide or fragment and the Col 11A1 polypeptide or fragment substrate in the absence of the test agent). Generally a plurality of assay mixtures is run in parallel with different agent concentrations to obtain a differential response to the various concentrations. Typically, one of these concentrations serves as a negative control, i.e. at zero concentration or below the level of detection.

A variety of other reagents may be included in the screening assay. These include reagents like salts, neutral proteins, e.g. albumin, detergents, etc., including agents that are used to facilitate optimal enzyme activity and/or reduce non-specific or background activity. Reagents that improve the efficiency of the assay, such as protease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity, but may also be optimized to facilitate rapid high-throughput screening. Typically between 0.1 hour and 1 hour will be sufficient.

A test agent that increases or decreases activity of the Col 11A1 polypeptide or fragment is a candidate agent for treating a disease or condition related to bone mineralization. For example, a test agent that increases or decreases activity of a Col 11A1 polypeptide or fragment polypeptide by at least about 20%, at least about 25%, at least about 50%, at least about 75%, at least about 2-fold, at least about 2.5-fold, at least about 5-fold, at least about 10-fold, or more than 10-fold, compared to the enzymatic activity of the Col 11A1 or fragment polypeptide in the absence of the test agent, is considered a candidate agent for treating a disease or condition related to oxidative stress and/or oxidative damage.

In some embodiments, a test compound of interest has an EC₅₀ of from about 1 nM to about 1 mM, e.g., from about 1 nM to about 10 nM, from about 10 nM to about 15 nM, from about 15 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, from about 450 nM to about 500 nM, from about 500 nM to about 750 nM, from about 750 nM to about 1 μM, from about 1 μM to about 10 μM, from about 10 μM to about 25 μM, from about 25 μM to about 50 μM, from about 50 μM to about 75 μM, from about 75 μM to about 100 μM, from about 100 μM to about 250 μM, from about 250 μM to about 500 μM, or from about 500 μM to about 1 mM.

In many embodiments, the screening method is carried out in vitro, in a cell-free assay. In some embodiments, the in vitro cell-free assay will employ a purified Col 11A1 or fragment thereof, where “purified” refers to free of contaminants or any other undesired components. Purified Col 11A1 or fragment thereof that is suitable for a subject screening method is at least about 50% pure, at least about 60% pure, at least about 70% pure, at least about 75% pure, at least about 80% pure, at least about 85% pure, at least about 90% pure, at least about 95% pure, at least about 98% pure, at least about 99% pure, or greater than 99% pure.

Purified Col 11A1 or fragment thereof polypeptide will in some embodiments be stabilized by addition of one or more stabilizing agents, to maintain enzymatic activity. In some embodiments, a solution of purified Col 11A1 or fragment thereof polypeptide comprises an aqueous solution comprising a Col 11A1 or fragment thereof polypeptide and from about 10% to about 50% glycerol, e.g., from about 10% to about 15%, from about 15% to about 20%, from about 20% to about 25%, from about 25% to about 30%, from about 30% to about 35%, from about 35% to about 40%, from about 40% to about 45%, or from about 45% to about 50% glycerol. In some embodiments, a solution comprising a Col 11A1 or fragment thereof polypeptide further comprises one or more of a chelating agent (e.g., EDTA or EGTA); salts such as NaCl, MgCl₂, KCl, and the like; buffers, such as a Tris buffer, phosphate-buffered saline, sodium pyrophosphate buffer, and the like; one or more protease inhibitors; and the like.

A Col 11A1 or fragment thereof polypeptide suitable for use in a subject screening method can comprise an amino acid sequence having at least about 85%, at least about 90%, at least about 95%, at least about 98%, at least about 99%, or 100%, amino acid sequence identity to the amino acid sequence of a Col 11A1 or fragment thereof polypeptide as disclosed herein, SEQ ID NOS 1, 2, or 3.

A Col 11A1 or fragment thereof suitable for use in a subject screening method can comprise an amino acid sequence having at least about 85%, at least about 90%, at least about 95%, at least about 98%, at least about 99%, or 100%, amino acid sequence identity to the amino acid sequence of a Col 11A1 polypeptide or fragment thereof.

A Col 11A1 or fragment thereof is readily prepared in a variety of host cells such as unicellular microorganisms, or cells of multicellular organisms grown in in vitro culture as unicellular entities. Suitable host cells include bacterial cells such as Escherichia coli; yeast cells such as Saccharomyces cerevisiae, Pichia pastoris, Hansenula polymorpha, Kluyveromyces lactis, Yarrowia lipolytica, Candida utilis, Schizosaccharomyces pombe, and the like; insect cells such as Drosophila melanogaster cells; amphibian cells such as Xenopus cells; mammalian cells, such as CHO cells, 3T3 cells, and the like.

In some embodiments, the in vitro cell-free assay will employ a fusion protein, comprising a Col 11A1 or fragment thereof fused in-frame to a fusion partner. In some embodiments, the fusion partner is attached to the amino terminus of the Col 11A1 or fragment thereof. In other embodiments, the fusion partner is attached to the carboxyl terminus of the Col 11A1 or fragment thereof. In other embodiments, the fusion partner is fused in-frame to the Col 11A1 or fragment thereof at a location internal to the Col 11A1 or fragment thereof. Suitable fusion partners include immunological tags such as epitope tags, including, but not limited to, hemagglutinin, FLAG, and the like; proteins that provide for a detectable signal, including, but not limited to, fluorescent proteins, enzymes (e.g., β-galactosidase, luciferase, horse radish peroxidase, etc.), and the like; polypeptides that facilitate purification or isolation of the fusion protein, e.g., metal ion binding polypeptides such as 6His tags (e.g., Col 11A1 or fragment/6His), glutathione-S-transferase, and the like; polypeptides that provide for subcellular localization; and polypeptides that provide for secretion from a cell.

In some embodiments, the fusion partner is an epitope tag. In some embodiments, the fusion partner is a metal chelating peptide. In some embodiments, the metal chelating peptide is a histidine multimer, e.g., (His)₆. In some embodiments, a (His)₆ multimer is fused to the amino terminus of a Col 11A1 or fragment thereof 2 polypeptide; in other embodiments, a (His)₆ multimer is fused to the carboxyl terminus of a Col 11A1 or fragment thereof. The (His)6-Col 11A1 or fragment fusion protein is purified using any of a variety of available nickel affinity columns (e.g. His-bind resin, Novagen).

In some embodiments, a subject screening method is carried out in vitro in a cell, e.g., a cell grown in cell culture as a unicellular entity. Suitable cells include, e.g., eukaryotic cells, e.g., mammalian cells such as CHO cells 293 cells, 3R3 cells, and the like.

### Decreasing Col 11A1 Activity

In some embodiments it may be desirable to decrease bone mineralization in a subject. In cases where mineralization must be controlled, for example in the healing of difficult bone fractures or in the case of large osteochondral defects that will not heal without intervention, agents that decrease expression or activity of specific isoforms or inclusion of specific domains of the Col 11A1 protein may be useful to optimize the mineralization and healing process. For example, a biomaterial scaffold for the repair of cartilage or blood vessels should not mineralize. Exemplary agents that decrease the expression and/or activity of Col 11A1 include inhibitory nucleic acids and inhibitory amino acids, as well as inhibitory molecules such as small molecules.

In one aspect, the agent that decreases the expression is an inhibitory nucleic acid molecule, wherein administration of the inhibitory nucleic acid molecule selectively decreases the expression of Col 11A1, for example, Col 11A1 comprising exon 6A and/or exon 8. The term “inhibitory nucleic acid molecule” means a single stranded or double-stranded RNA or DNA, specifically RNA, such as triplex oligonucleotides, ribozymes, aptamers, small interfering RNA including siRNA (short interfering RNA) and shRNA (short hairpin RNA), antisense RNA, or a portion thereof, or an analog or mimetic thereof, that is capable of reducing or inhibiting the expression of a target gene or sequence. Inhibitory nucleic acids can act by, for example, mediating the degradation or inhibiting the translation of mRNAs which are complementary to the interfering RNA sequence. An inhibitory nucleic acid, when administered to a mammalian cell, results in a decrease (e.g., by 5%, 10%, 25%, 50%, 75%, or even 90-100%) in the expression (e.g., transcription or translation) of a target sequence.

Typically, a nucleic acid inhibitor comprises or corresponds to at least a portion of a target nucleic acid molecule, or an ortholog thereof, or comprises at least a portion of the complementary strand of a target nucleic acid molecule. Inhibitory nucleic acids may have substantial or complete identity to the target gene or sequence, or may include a region of mismatch (i.e., a mismatch motif). The sequence of the inhibitory nucleic acid can correspond to the full-length target gene, or a subsequence thereof. In one aspect, the inhibitory nucleic acid molecules are chemically synthesized.

The specific sequence utilized in design of the inhibitory nucleic acids is a contiguous sequence of nucleotides contained within the expressed gene message of the target. Factors that govern a target site for the inhibitory nucleic acid sequence include the length of the nucleic acid, binding affinity, and accessibility of the target sequence. Sequences may be screened in vitro for potency of their inhibitory activity by measuring inhibition of target protein translation and target related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

One class of inhibitory nucleic acids includes antisense oligonucleotides. The antisense oligonucleotides may include oligonucleotides that are composed of naturally-occurring nucleobases, sugars and covalent internucleoside (backbone) linkages as well as oligonucleotides having non-naturally-occurring nucleobases, sugars and covalent internucleoside (backbone) linkages. Non-naturally-occurring portions of the antisense molecules may be preferred, as these portions may endow the antisense molecules with desirable properties such as, for example, enhanced affinity for nucleic acid target and increased stability in the presence of nucleases. Throughout the disclosure, a nucleotide having any non-naturally occurring portion is referred to as a modified nucleotide (and the term modified nucleotide is used for convenience, including when such modification alters the structure of the nucleotide so that is technically no longer a nucleotide, e.g., it is a nucleic acid or nucleoside).

Nucleosides are base-sugar combinations. Normally, the base portion of a nucleoside is a heterocyclic base, e.g., a purine or a pyrimidines base. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

In some embodiments, the antisense oligonucleotides of the present disclosure include oligonucleotides containing modified backbones or non-natural internucleoside linkages. In some embodiments, the oligonucleotides having modified backbones include those that retain a phosphorus atom in the backbone. In other embodiments, the oligonucleotides having modified backbones include those that do not have a phosphorus atom in the backbone.

In some embodiments, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In some embodiments of the present disclosure, the oligonucleotide backbone includes, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

In some embodiments, in modified oligonucleotide, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA nucleotides include, but are not limited to, U.S. Pat. Nos. 5,539,082; 5,714,331; and 5,719,262, each of which is herein incorporated by reference. Further teaching of PNA nucleotides can be found in Nielsen et al., Science, 1991, 254, 1497-1500.

In some embodiments of the present disclosure are oligonucleotides with phosphorothioate backbones and oligonucleosides with heteroatom backbones, such as —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P(═O)(OH)—O—CH₂—], and the amide backbones of the above referenced U.S. Pat. No. 5,602,240, or the morpholino backbone structures of the above-referenced U.S. Pat. No. 5,034,506.

Modified oligonucleotides may also contain one or more substituted sugar moieties. In some embodiments, the oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In particular embodiments, the oligonucleotides comprise O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Other embodiments include antisense molecules comprising 2′-dimethylaminooxyethoxy, i.e., a O(CH₂)₂ON(CH₃)₂ group, also known as 2′-DMAOE or 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

In some embodiments, the antisense oligonucleotides of the present disclosure include an alkoxyalkoxy group, e.g., 2′-methoxyethoxy (2′-O—C₂CH₂OCH₃, also known as 2′-O-(2-methoxyethyl) or 2′-MOE) (Martin et al., Helv. Chim. Acta, 1995, 78, 486-504). In one embodiment, the antisense oligonucleotides of the present disclosure include 2′-MOE. In some embodiments, the antisense oligonucleotides comprise 1-10 MOE nucleotides. In other embodiments, the antisense oligonucleotides comprise 2-7 MOE nucleotides. In other embodiments, the antisense oligonucleotides comprise 3-6 MOE nucleotides.

In some embodiments, the antisense oligonucleotides of the present disclosure include a nucleotide analog having a constrained furanose ring conformation, such as Locked Nucleic Acids (LNAs). In LNAs, a 2′-hydroxyl group is linked to the 3′ or 4′ carbon atom of the sugar ring thereby forming a bicyclic sugar moiety. In some embodiments, the linkage in the LNA is a methylene (—CH₂—)_{g}roup bridging the 2′ oxygen atom and the 4′ carbon atom wherein n is 1 or 2. LNAs and preparation thereof are described in WO 98/39352 and WO 99/14226. In some embodiments, the antisense oligonucleotides comprise 1-10 LNA nucleotides. In other embodiments, the antisense molecules comprise 2-7 LNA nucleotides. In other embodiments, the antisense molecules comprise 3-6 LNA nucleotides.

In other embodiments of the antisense oligonucleotides of the present disclosure, modifications to the antisense molecules include 2′-methoxy (2′-O—CH₃), 2′-aminopropoxy (2′-OCH₂CH₂CH₂NH₂), 2′-allyl(2′-CH₂—CH═CH₂), 2′-O-allyl(2′-O—CH₂—CH═CH₂) and 2′-fluoro (2′-F). The 2′-modification may be in the arabino (up) position or ribo (down) position. An example of a 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920.

The antisense oligonucleotides of the present disclosure may also include nucleobase (often referred to in the art simply as “base”) modifications or substitutions. An “unmodified” or “natural” nucleobase, as used herein, includes the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the disclosure. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941.

It is not necessary for all positions in a given compound to be uniformly modified, and in fact more than one of the aforementioned modifications may be incorporated in a single compound or even at a single nucleoside within an oligonucleotide. The present disclosure also includes antisense oligonucleotides which are chimeric compounds. “Chimeric” antisense compounds or “chimeras,” in the context of this disclosure, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense oligonucleotides of the disclosure may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

A “gapmer” is defined as an oligomeric compound, generally an oligonucleotide, having a 2′-deoxyoligonucleotide region flanked by non-deoxyoligonucleotide segments. The central region is referred to as the “gap.” The flanking segments are referred to as “wings.” While not wishing to be bound by theory, the gap of the gapmer presents a substrate recognizable by RNaseH when bound to the RNA target whereas the wings do not provide such a substrate but can confer other properties such as contributing to duplex stability or advantageous pharmacokinetic effects. Each wing can be one or more non-deoxyoligonucleotide monomers (if one of the wings has zero non-deoxyoligonucleotide monomers, a “hemimer” is described). In one embodiment, the gapmer is a ten deoxyribonucleotide gap flanked by five non-deoxyribonucleotide wings. This is referred to as a 5-10-5 gapmer. In other embodiments, the gapmer is an eight deoxyribonucleotide gap flanked by three non-deoxyribonucleotide wings. This is referred to as a 3-8-3 gapmer. In other embodiments, the gapmer is a ten deoxyribonucleotide gap flanked by three non-deoxyribonucleotide wings. This is referred to as a 3-10-3 gapmer. Other configurations are readily recognized by those skilled in the art, such as a 3-7-3 gapmer.

In some embodiments, the gapmer described above comprises LNA and MOE nucleotides. In some embodiments, the gapmer comprises 1-10 LNA and/or MOE nucleotides. In some embodiments, the gapmer comprises 2-7 LNA and/or MOE nucleotides. In other embodiments, the gapmer comprises 3-6 MOE and/or LNA nucleotides. In some embodiments the flanking blocks of ribonucleotides comprise LNA and/or MOE nucleotides.

In some embodiments, the gapmers described above induce RNase H degradation of the target RNA nucleotide. In other embodiments, the gapmers induce degradation of the target RNA nucleotide by means of an RNase H-independent pathway. In some embodiments, the gapmers prevents the binding of a protein, to a DNA or RNA sequence.

In some embodiments, the gapmers induce degradation of the target RNA molecule, and also sterically inhibit the binding of a protein.

In some embodiments, the antisense oligonucleotide is a gapmer that binds to expanded CUG repeats in an RNA molecule. In some embodiments, the gapmer comprises a sequence that is at least 60%, 65%, 70%, 80%, 85%, 90%, 91%, 92%, 93%, 94%, 95%, 96%, 97%, 98%, 99% or 100% identical to any one of SEQ ID NOs: 1-3.

In some embodiments, the antisense oligonucleotide is a morpholino molecule that sterically blocks the binding of a protein or nucleic acid to a target RNA or DNA sequence. In some embodiments, the morpholino also triggers degradation of the target RNA or DNA sequence. In some embodiments, the morpholino molecule binds to RNA and prevents the binding of Muscle blind like protein, (MBNL1) to the RNA molecule. In some embodiments, the MBNL1 protein that is prevented from binding to the RNA molecule is free to bind to other RNA molecule substrates. In some embodiments, the morpholino molecule comprises 20-30 nucleotides. In other embodiments, the morpholino molecule comprises 23-27 nucleotides. In other embodiments, the morpholino molecule comprises 25 nucleotides. In some embodiments, the morpholino binds CUG repeats in an RNA molecule. In particular embodiments, the morpholino binds to CUG repeats in a mutant RNA sequence.

In some embodiments, the antisense oligonucleotides of the present disclosure are molecules including 2′-O-methyl (2′-OMe) and/or phosphorothioate modifications and that specifically trigger the degradation of an RNA molecule. In some embodiments, these molecules include 2′-O-methyl (2′-OMe) and phosphorothioate modifications. In some embodiments, these molecules induce degradation of a target RNA sequence, by means an RNaseH mediated degradation or by other than RNase H degradation.

Representative modifications are depicted below. The disclosure contemplates antisense oligonucleotides comprising nucleotides modified, as depicted below, including antisense oligonucleotides including combinations of the depicted chemistries (e.g., antisense oligonucleotides including any one or more of the depicted modifications).

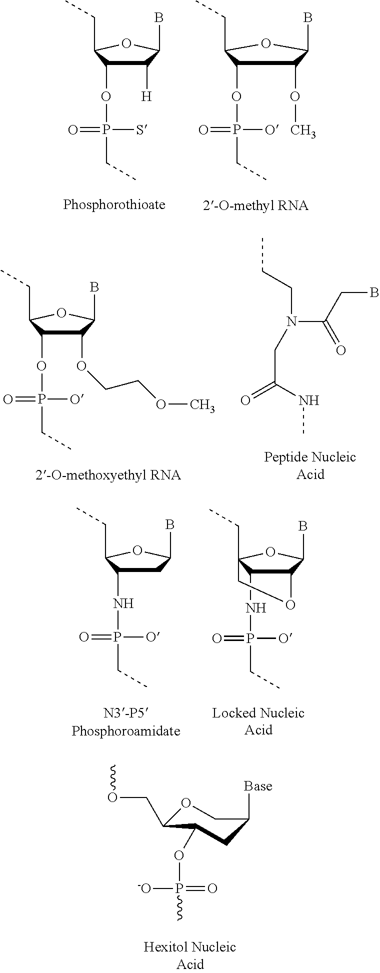

For all of the foregoing, it should be appreciated that certain antisense oligonucleotides promote RNaseH mediated degradation following hybridization to target. However, even for such antisense oligonucleotides, such capability does not mean or imply that this is the sole mechanism by which the antisense oligonucleotide functions.

In another embodiment, the inhibitory molecules may be short interfering RNA. Short interfering (si) RNA technology (also known as RNAi) generally involves degradation of an mRNA of a particular sequence induced by double-stranded RNA (dsRNA) that is homologous to that sequence, thereby “interfering” with expression of the corresponding gene. A selected gene may be repressed by introducing a dsRNA which corresponds to all or a substantial part of the mRNA for that gene. Without being held to theory, it is believed that when a long dsRNA is expressed, it is initially processed by a ribonuclease III into shorter dsRNA oligonucleotides of as few as 21 to 22 base pairs in length. Accordingly, siRNA may be affected by introduction or expression of relatively short homologous dsRNAs. Exemplary siRNAs have sense and antisense strands of about 21 nucleotides that form approximately 19 nucleotides of double stranded RNA with overhangs of two nucleotides at each 3′ end.

siRNA has proven to be an effective means of decreasing gene expression in a variety of cell types. siRNA typically decreases expression of a gene to lower levels than that achieved using antisense techniques, and frequently eliminates expression entirely. In mammalian cells, siRNAs are effective at concentrations that are several orders of magnitude below the concentrations typically used in antisense experiments.

The double stranded oligonucleotides used to effect RNAi are specifically less than 30 base pairs in length, for example, about 29, 28, 27, 26, 25, 24, 23, 22, 21, 20, 19, 18, or 17 base pairs or less in length, and contain a segment sufficiently complementary to the target mRNA to allow hybridization to the target mRNA. Optionally, the dsRNA oligonucleotide includes 3′ overhang ends. Exemplary 2-nucleotide 3′ overhangs are composed of ribonucleotide residues of any type and may be composed of 2′-deoxythymidine residues, which lowers the cost of RNA synthesis and may enhance nuclease resistance of siRNAs in the cell culture medium and within transfected cells. Exemplary dsRNAs are synthesized chemically or produced in vitro or in vivo using appropriate expression vectors. Longer RNAs may be transcribed from promoters, such as T7 RNA polymerase promoters, known in the art.

Longer dsRNAs of 50, 75, 100, or even 500 base pairs or more also may be utilized in certain embodiments. Exemplary concentrations of dsRNAs for effecting RNAi are about 0.05 nM, 0.1 nM, 0.5 nM, 1.0 nM, 1.5 nM, 25 nM, or 100 nM, although other concentrations may be utilized depending upon the nature of the cells treated, the gene target and other factors readily identifies by one of ordinary skill in the art.

Compared to siRNA, shRNA offers advantages in silencing longevity and delivery options. Vectors that produce shRNAs, which are processed intracellularly into short duplex RNAs having siRNA-like properties provide a renewable source of a gene-silencing reagent that can mediate persistent gene silencing after stable integration of the vector into the host-cell genome. Furthermore, the core silencing ‘hairpin’ cassette can be readily inserted into retroviral, lentiviral, or adenoviral vectors, facilitating delivery of shRNAs into a broad range of cell types.

A hairpin can be organized in either a left-handed hairpin (i.e., 5′-antisense-loop-sense-3′) or a right-handed hairpin (i.e., 5′-sense-loop-antisense-3′). The shRNA may also contain overhangs at either the 5′ or 3′ end of either the sense strand or the antisense strand, depending upon the organization of the hairpin. If there are any overhangs, they are specifically on the 3′ end of the hairpin and include 1 to 6 bases. The overhangs can be unmodified, or can contain one or more specificity or stabilizing modifications, such as a halogen or O-alkyl modification of the 2′ position, or internucleotide modifications such as phosphorothioate, phosphorodithioate, or methylphosphonate modifications. The overhangs can be ribonucleic acid, deoxyribonucleic acid, or a combination of ribonucleic acid and deoxyribonucleic acid.

Additionally, a hairpin can further comprise a phosphate group on the 5′-most nucleotide. The phosphorylation of the 5′-most nucleotide refers to the presence of one or more phosphate groups attached to the 5′ carbon of the sugar moiety of the 5′-terminal nucleotide. Specifically, there is only one phosphate group on the 5′ end of the region that will form the antisense strand following Dicer processing. In one exemplary embodiment, a right-handed hairpin can include a 5′ end (i.e., the free 5′ end of the sense region) that does not have a 5′ phosphate group, or can have the 5′ carbon of the free 5′-most nucleotide of the sense region being modified in such a way that prevents phosphorylation. This can be achieved by a variety of methods including, but not limited to, addition of a phosphorylation blocking group (e.g., a 5′-O-alkyl group), or elimination of the 5′-OH functional group (e.g., the 5′-most nucleotide is a 5′-deoxy nucleotide). In cases where the hairpin is a left-handed hairpin, preferably the 5′ carbon position of the 5′-most nucleotide is phosphorylated.

Hairpins that have stem lengths longer than 26 base pairs can be processed by Dicer such that some portions are not part of the resulting siRNA that facilitates mRNA degradation. Accordingly the first region, which may include sense nucleotides, and the second region, which may include antisense nucleotides, may also contain a stretch of nucleotides that are complementary (or at least substantially complementary to each other), but are or are not the same as or complementary to the target mRNA. While the stem of the shRNA can include complementary or partially complementary antisense and sense strands exclusive of overhangs, the shRNA can also include the following: (1) the portion of the molecule that is distal to the eventual Dicer cut site contains a region that is substantially complementary/homologous to the target mRNA; and (2) the region of the stem that is proximal to the Dicer cut site (i.e., the region adjacent to the loop) is unrelated or only partially related (e.g., complementary/homologous) to the target mRNA. The nucleotide content of this second region can be chosen based on a number of parameters including but not limited to thermodynamic traits or profiles.

Modified shRNAs can retain the modifications in the post-Dicer processed duplex. In exemplary embodiments, in cases in which the hairpin is a right handed hairpin (e.g., 5′-S-loop-AS-3′) containing 2-6 nucleotide overhangs on the 3′ end of the molecule, 2′-O-methyl modifications can be added to nucleotides at position 2, positions 1 and 2, or positions 1, 2, and 3 at the 5′ end of the hairpin. Also, Dicer processing of hairpins with this configuration can retain the 5′ end of the sense strand intact, thus preserving the pattern of chemical modification in the post-Dicer processed duplex. Presence of a 3′ overhang in this configuration can be particularly advantageous since blunt ended molecules containing the prescribed modification pattern can be further processed by Dicer in such a way that the nucleotides carrying the 2′ modifications are removed. In cases where the 3′ overhang is present/retained, the resulting duplex carrying the sense-modified nucleotides can have highly favorable traits with respect to silencing specificity and functionality. Examples of exemplary modification patterns are described in detail in U.S. Patent Publication No. 20050223427 and International Patent Publication Nos. WO 2004/090105 and WO 2005/078094, the disclosures of each of which are incorporated by reference herein in their entirety.

shRNA may comprise sequences that were selected at random, or according to a rational design selection procedure. For example, rational design algorithms are described in International Patent Publication No. WO 2004/045543 and U.S. Patent Publication No. 20050255487, the disclosures of which are incorporated herein by reference in their entireties. Additionally, it may be desirable to select sequences in whole or in part based on average internal stability profiles (“AISPs”) or regional internal stability profiles (“RISPs”) that may facilitate access or processing by cellular machinery.

Ribozymes are enzymatic RNA molecules capable of catalyzing specific cleavage of mRNA, thus preventing translation. The mechanism of ribozyme action involves sequence specific hybridization of the ribozyme molecule to complementary target RNA, followed by an endonucleolytic cleavage event. The ribozyme molecules specifically include (1) one or more sequences complementary to a target mRNA, and (2) the well-known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site-specific recognition sequences can be used to destroy target mRNAs, hammerhead ribozymes may alternatively be used. Hammerhead ribozymes cleave mRNAs at locations dictated by flanking regions that form complementary base pairs with the target mRNA. Specifically, the target mRNA has the following sequence of two bases: 5′-UG-3′. The construction and production of hammerhead ribozymes is well known in the art and is described more fully in U.S. Pat. No. 5,633,133, the contents of which are incorporated herein by reference.

Gene targeting ribozymes may contain a hybridizing region complementary to two regions of a target mRNA, each of which is at least 5, 6, 7, 8, 9, 10, 11, 12, 13, 14, 15, 16, 17, 18, 19, or 20 contiguous nucleotides (but which need not both be the same length).

Hammerhead ribozyme sequences can be embedded in a stable RNA such as a transfer RNA (tRNA) to increase cleavage efficiency in vivo. In particular, RNA polymerase III-mediated expression of tRNA fusion ribozymes is well known in the art. There are typically a number of potential hammerhead ribozyme cleavage sites within a given target cDNA sequence. Specifically, the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the target mRNA would allow the selective targeting of one or the other target genes.

Ribozymes also include RNA endoribonucleases (“Cech-type ribozymes”) such as the one which occurs naturally in Tetrahymena thermophile, described in International Patent Publication No. WO 88/04300. The Cech-type ribozymes have an eight base pair active site which hybridizes to a target RNA sequence where after cleavage of the target RNA takes place. In one embodiment, Cech-type ribozymes target eight base-pair active site sequences that are present in a target gene or nucleic acid sequence.

Ribozymes can be composed of modified oligonucleotides (e.g., for improved stability, targeting, etc.) and can be chemically synthesized or produced through an expression vector. Because ribozymes, unlike antisense molecules, are catalytic, a lower intracellular concentration is required for efficiency. Additionally, in certain embodiments, a ribozyme may be designed by first identifying a sequence portion sufficient to cause effective knockdown by RNAi. Portions of the same sequence may then be incorporated into a ribozyme.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body. Nucleic acid molecules to be used in triple helix formation for the inhibition of transcription are specifically single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the target sequences that can be targeted for triple helix formation may be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

Inhibitory nucleic acids can be administered directly or delivered to cells by transformation or transfection via a vector, including viral vectors or plasmids, into which has been placed DNA encoding the inhibitory oligonucleotide with the appropriate regulatory sequences, including a promoter, to result in expression of the inhibitory oligonucleotide in the desired cell. Known methods include standard transient transfection, stable transfection and delivery using viruses ranging from retroviruses to adenoviruses. Delivery of nucleic acid inhibitors by replicating or replication-deficient vectors is contemplated. Expression can also be driven by either constitutive or inducible promoter systems. In other embodiments, expression may be under the control of tissue or development-specific promoters.

Vectors may be introduced by transfection using carrier compositions such as Lipofectamine 2000 (Life Technologies) or Oligofectamine™ (Life Technologies). Transfection efficiency may be checked using fluorescence microscopy for mammalian cell lines.

The effectiveness of the inhibitory oligonucleotide may be assessed by any of a number of assays, including reverse transcriptase polymerase chain reaction or Northern blot analysis to determine the level of existing Col 11A1, or Western blot analysis using antibodies which recognize the Col 11A1, after sufficient time for turnover of the endogenous pool after new protein synthesis is repressed.

Further included are pharmaceutical compositions comprising a pharmaceutically acceptable carrier/excipient and a small interfering RNA, the small interfering RNA comprising 19 to 29 nucleotides that are substantially complementary to a sequence of 19 to 29 nucleotides of Col 11A1.

### Polypeptide Antagonist Agents

Methods of the present invention encompasses Col 11A1 antagonist agents that are polypeptides. In one embodiment, a polypeptide antagonist agent is a Col 11A1 antibody or fragment thereof that immunospecifically binds Col 11A1 and antagonizes Col 11A1. In another embodiment, a polypeptide antagonist agent is a Col 11A1 binding-partner or fragment thereof that is capable of binding Col 11A1 and antagonizing Col 11A1 (e.g., regulates osteoblast differentiation, bone mineralization, and/or decreases a pathology-causing phenotype).

### Antibodies as Polypeptide Antagonist Agents

In one embodiment, Col 11A1 antagonist agents of the invention encompass antibodies (preferably, monoclonal antibodies) or fragments thereof that immunospecifically bind to Col 11A1 and regulate Col 11A1 mediated osteoblast activity, decrease a pathology-causing phenotype (e.g., regulation of osteoblast differentiation, modulation of bone mineralization) and/or bind Col 11A1 with a K_{off} of less than 3×10⁻³ s⁻¹. In one embodiment, the antibody binds to the NTD of Col 11A1 (e.g., at an epitope either within or outside of the Col 11A1 variable region) and, preferably, also antagonize Col 11A1, e.g., regulates Col 11A1-mediated osteoblast differentiation and, preferably, regulates bone mineralization. In other embodiments, the antibodies inhibit or reduce a pathology-causing phenotype in the presence of another agent used in non-neoplastic hyperproliferative cell or excessive cell accumulation disorder therapy. In another embodiment, the antibody binds to the NTD of Col 11A1, preferably with a K_{off} of less than 1×10⁻³ s⁻¹, more preferably less than 3×10⁻³ s⁻¹. In other embodiments, the antibody binds to Col 11A1 with a K_{off} of less than 10⁻³ s⁻¹, less than 5×0⁻³ s⁻¹, less than 10⁻⁴ s.⁻¹, less than 5×10⁻⁴ s⁻¹, and the like.

In one embodiment, the antibody is commercially available from any of a number of sources including ORIGENE, and abcam.

Antibodies of the invention include, but are not limited to, synthetic antibodies, monoclonal antibodies, recombinantly produced antibodies, multispecific antibodies (including bi-specific), human antibodies, humanized antibodies, chimeric antibodies, synthetic antibodies, intrabodies, single-chain Fvs (scFv) (e.g., monospecific, bi-specific, etc.), Fab fragments, F(ab′) fragments, disulfide-linked Fvs (sdFv), and anti-idiotypic (anti-Id) antibodies, intrabodies, and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to Col 11A1 and is an antagonist of Col 11A1 and/or inhibits or reduces a pathology-causing cell phenotype and/or binds Col 11A1 with a K_{off} of less than 3×10⁻³ s⁻¹. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The present invention encompasses single domain antibodies, including camelized single domain antibodies (see e.g., Muyldermans et al., 2001, Trends Biochem. Sci. 26:230; Nuttall et al. 2000, Cur. Pharm. Biotech. 1; 253; Reichmann and Muyldermans, 1999, J. Immunol Meth. 231:25; International Patent Publication Nos. WO 94/04678 and WO 94/25591; U.S. Pat. No. 6,005,079; which are incorporated herein by reference in their entireties). In one embodiment, the present invention provides single domain antibodies comprising two V_{H} domains having the amino acid sequence of any of the V_{H} domains of the Col 11A1 antagonistic antibodies, or any other antagonistic antibody that increases Col 11A1 cytoplasmic tail phosphorylation, increases Col 11A1 autophosphorylation, reduces Col 11A1 activity (other than autophosphorylation), decreases a pathology-causing cell phenotype, or binds Col 11A1 with a low K_{off} rate) with modifications such that single domain antibodies are formed. In another embodiment, the present invention also provides single domain antibodies comprising two V_{H} domains comprising one or more of the V_{H} CDRs from any of the Col 11A1 antagonistic antibodies or any other antagonistic antibody that increases Col 11A1 cytoplasmic tail phosphorylation, increases Col 11A1 autophosphorylation, reduces Col 11A1 activity (other than autophosphorylation), decreases a pathology-causing cell phenotype, or binds Col 11A1 with a low K_{off} rate).

Antibodies of the invention include Col 11A1 intrabodies. Antibody antagonistic agents of the invention that are intrabodies immunospecifically bind Col 11A1 and agonize Col 11A1. In a more specific embodiment, an intrabody of the invention immunospecifically binds to the intracellular domain of Col 11A1 and causes Col 11A1 degradation. In another specific embodiment, the intrabody binds to the intracellular domain of Col 11A1 and decreases and/or slows cell proliferation, growth and/or survival of a Col 11A1-expressing cell. In another specific embodiment, the intrabody binds to the intracellular domain of Col 11A1 and maintains/reconstitutes the integrity of an epithelial cell layer.

The antibodies used in the methods of the invention may be from any animal origin including birds and mammals (e.g., human, murine, donkey, sheep, rabbit, goat, guinea pig, camel, horse, or chicken). In a most preferred embodiment, the antibody is human or has been humanized. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multispecificity. Multispecific antibodies may immunospecifically bind to different epitopes of a Col 11A1 polypeptide or may immunospecifically bind to both a Col 11A1 polypeptide as well as a heterologous epitope, such as a heterologous polypeptide or solid support material. See, e.g., International Patent Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Col 11A1 Regulates Bone Microarchitecture

### 1. Introduction

The skeleton forms by a combination of endochondral and intramembranous ossification. Fetal long bone formation proceeds by the process of endochondral ossification in which mesenchymal stem cells condense into an anlagen, or cartilage model, then subsequently undergo chondrogenesis. Chondrocytes secrete a cartilage-specific ECM and undergo longitudinal proliferation resulting in the elongation of long bones. Undifferentiated mesenchymal cells peripheral to the cartilage anlagen develop directly into the bony collar through the process of intramembranous bone formation that does not transition through a cartilage intermediate.

Chondrocytes at the diaphysis of the developing long bone undergo further maturation and hypertrophy, followed by an exit from the cell cycle.[1,2] Hypertrophic chondrocytes expressing collagen type X, alkaline phosphatase, Runx2, osteopontin, and osteocalcin stimulate the calcification of cartilage in the hypertrophic zone of the growth plate.[3,4] Ossification begins with invasion of the calcified hypertrophic cartilage by capillaries from the perichondrium, is followed by the apoptosis of terminal hypertrophic chondrocytes and the degradation of cartilage matrix; ossification ends with the deposition of bone matrix by osteoblasts on residual calcified cartilage matrix that gives rise to the trabeculae of the primary spongiosa.[5-7]

Periosteal bone collar intramembranous ossification precedes the advancing front of endochondral ossification and is carried out by osteoblasts that arise from the mesenchymal cells surrounding the cartilaginous core. Appositional bone growth leads to an increase in diaphyseal diameter due to the deposition of new bone beneath the fibrous layer of the periosteum. The periosteal bone collar extends longitudinally toward both epiphyses, proximally and distally. Bone growth is accompanied by the enlargement of the marrow cavity due to the destruction of bone tissue by osteoclasts [8,9] which dissolve the bone matrix.[10,11] The remodeling of bone matrix by osteoclasts supports the formation of a marrow cavity filled with vessels and hematopoietic cells.

Collagen type XI is a quantitatively minor but essential component of the ECM.[12] Collagen type XI nucleates the formation and regulates the diameter of heterotypic fibrils.[13-15] Col 11A1, Col 11a2 and Col2a1 form the triple helical collagen XI in cartilage [16] while alternative combinations are formed in bone, which include the minor fibrillar collagen alpha chains of types V and XI. Minor fibrillar collagens play essential roles in many tissues including heart valve, muscle, tendon, placenta, eye, and skin.[17-24]

Structurally, a triple helix is flanked by noncollagenous amino and carboxy terminal domains. Structural diversity arises in the amino terminal domains of the alpha chains of collagen type XI, Col 11A1, Col 11a2 and Col2a1, due to alternative splicing of the mRNA encoding each of the constituent alpha chains.[25-28] Col2a1 exists in one of two splice variants,[29] while numerous splice variants have been reported for Col 11a2.[19] In Col 11A1, alternative splicing of exons may generate up to eight possible protein isoforms, which are differentially expressed both temporally and spatially during development.[30] Col 11A1 [p6B] isoform is restricted to the cartilage periphery underlying the diaphyseal perichondrium during long bone development while the Col 11A1[p6A-7-8] isoform is associated with early chondrocyte differentiation through prechondrogenic mesenchyme and is later restricted to the articular surface.[26,30]

The importance of collagen XI in development is evident from the Col 11A1 functional knockout, the chondrodystrophic mouse (cho), which displays an autosomal recessive chondrodysplasia as a result of a point mutation in the Col 11A1 gene that causes a reading frame shift and results in a premature stop codon and mRNA instability; a functional knockout of Col 11A1 (Col 11A1^{-/-}).[31,32] In the absence of Col 11A1, an alternate triple helical molecule forms, consisting of Col 11a2 and Col5a1, which is unable to compensate for the functional deficiency caused by an absence of Col 11A1.[33]

The Col 11A1^{-/-} cartilage phenotype was previously characterized with deficiencies in chondrogenesis, epiphyseal cartilage structure, collagen fibrils, cleft palate, and auditory function.[34-39] Here we extend previous analysis and provide information on the mineralized skeleton and bone formation by histology and X-ray microtomography (micro-CT) to specifically assess bone formation in the absence of Col 11A1. The data presented here show that Col 11A1 depletion resulted in alteration to both trabecular and cortical bone. Characterization of the Col 11A1^{-/-} mouse mineralized tissue extends our previous in vitro osteoblast work to further explain the consequences of a loss of Col 11A1, influencing osteoblast differentiation and mineralization. These results provide new information on bone development and increase our understanding of human conditions involving a mutation of Col 11A1, including Stickler syndrome, Marshall syndrome, Wagner syndrome, and Fibrochondrogenesis.

### 2. Materials and Methods

### 2.1. Mice.

The embryos used in this study were provided by Dr. Robert Seegmiller (Brigham Young University). The mice were housed and euthanized as approved by the Institute of Animal Care and Use Committee of Brigham Young University. All embryos used in this study were at embryonic day 17.5. A total of six wildtype (WT) (+/+) and three homozygous cho (−/−) on a C57B16 background were analyzed.

### 2.2. Micro-CT Analysis.

Embryos were scanned with a SkyScan 1172 high resolution micro-CT scanner (MicroPhotonics, Aartselaar, Belgium) to generate data sets with a 1.7 μm³ isotropic voxel size using an acquisition protocol that consisted of X-ray tube settings of 60 kV and 250 μA, exposure time of 0.147 seconds, six-frame averaging, a rotation step of 0.300 degrees, and associated scan times were approximately 7 hours. Following scanning, a two-dimensional reconstruction stage was used to produce 6000 serial 4000×4000 pixel cross-sectional images. Three-dimensional models were reconstructed using a fixed threshold to analyze the mineralized bone phase using ImageVis 3D software (University of Utah, Center for Integrative Biomedical Computing, Salt Lake City, Utah). A light Gaussian filter (σ=1.0, kernel=3) to remove high frequency noise followed by an adaptive threshold was used to segment the 3D images, which were visually checked to confirm inclusion of complete volume of interest.

Gross geometric measurements were performed using Skyscan CT Analyzer (CTAn) software (MicroPhotonics, Aartselaar, Belgium). Comparisons of shape and cross-sectional area were conducted for long bones, ribs and spine. CTAn was used to determine trabecular thickness (Tb.Th), trabecular number (Tb.N), trabecular separation (Tb.Sp), degree of anisotropy (DA) and structure model index (SMI).[40-43] Trabecular thickness, number and separation measurements were performed on three-dimensional whole bone models of vertebrae, vertebral bodies and long bones in CTAn. Bone volume (BV) and bone surface (BS) were calculated based on the hexahedral marching cubes volume model of the binarized objects within the volume of interest and the faceted surface of the marching cubes volume model, respectively.[43] Total tissue volume (TV) was defined as the volume-of-interest, which in this case refers to the entire scanned sample. Trabecular bone volume fraction (BV/TV) was calculated from BV and TV values. The degree of anisotropy (DA) and structure model index (SMI) were calculated for long bones. Cross-sectional reconstructions were color-coded according to three density ranges: high density range (white), intermediate density range (blue), and low density range (green).

2.3. Trichrome stain.

Embryos were fixed in Bouin's solution [44] for 5 days and transferred to 70% ethanol for an additional 3 days Ribs and limbs were excised from mice, embedded in paraffin, and sectioned at 6 microns. The sections were stained according to Gomori's tri-chrome procedure, where aldehyde fuschin stained cartilage purple, fast green stained bone green, and phloxine B stained blood cells reddish pink.[45] Digital images were obtained with an Olympus BX51 photomicroscope.

### 2.4. Data Analysis.

Confidence intervals were determined at 95%. Differences between Col 11A1-deficient and WT embryos were identified as those for which the value for the Col 11A1-deficient embryo fell outside of the 95% confidence interval for the WT group.

### 3. Results

### 3.1. Changes to Embryonic Skeleton in the Absence of Col 11A1 Expression.

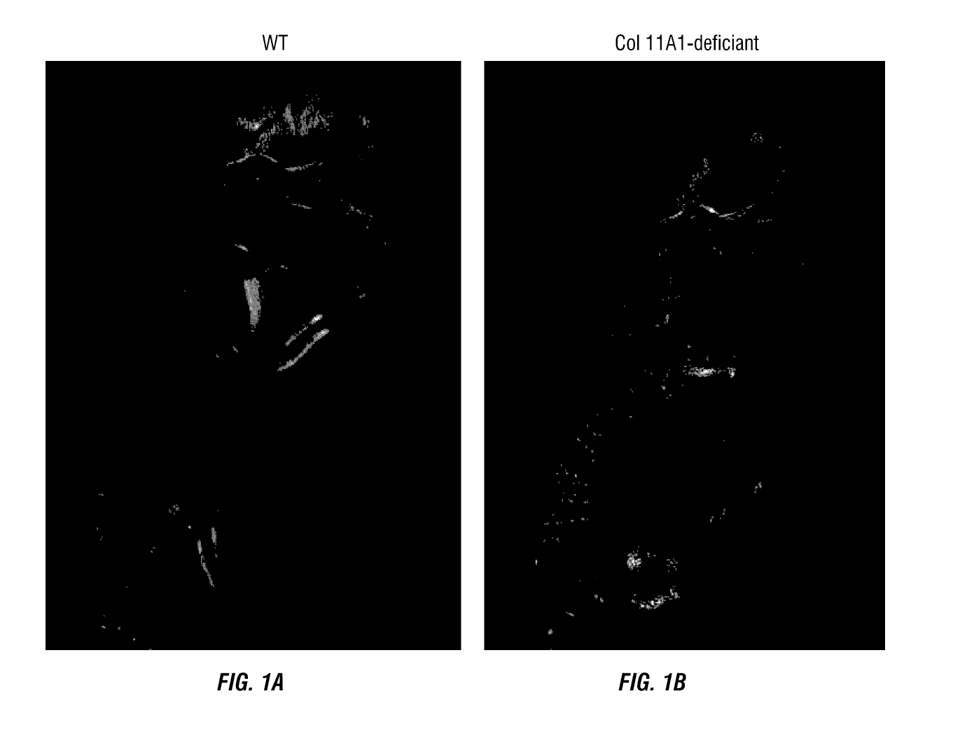

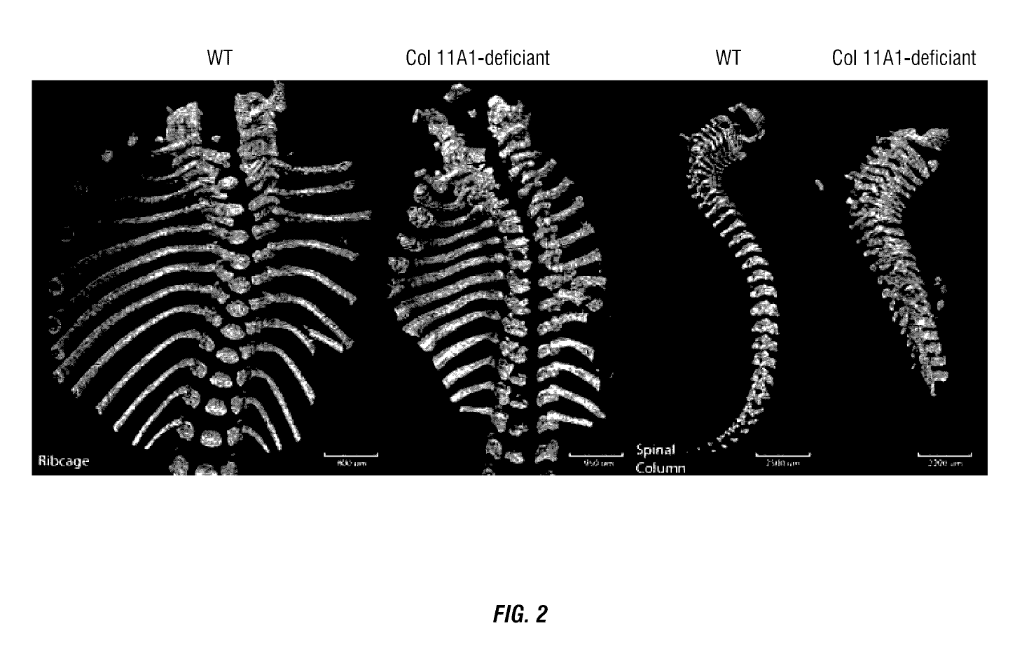

Micro-CT data was collected and three-dimensional models of mineralized skeleton were constructed for six WT and three Col 11A1-deficient mice. Overall anatomical features observed were consistent with those previously shown.[37] The skeletal deformities characteristic of the Col 11A1-deficient mouse included shortened, wider limb bones, shortened snout, small thoracic cage, and shortened spine. These were apparent in the three-dimensional reconstructions of mineralized skeleton (FIG. 1). To analyze the shortened spine and vertebrae in more detail, a reconstruction of the spine and ribs was made and is shown in FIG. 2. Analysis of three-dimensional reconstructions from X-ray micro-CT data revealed a decrease in separation between the vertebrae and an increase in the height of individual vertebrae in the Col 11A1-deficient mice compared to WT littermates. The extent of mineralization was reduced in the lower thoracic and lumbar vertebrae in the absence of Col 11A1. Mineralization of the lumbar vertebrae from the Col 11A1-deficient mice was below the limit of detection, and therefore was not visible in FIG. 2.

### 3.2 Analysis of Vertebrae.

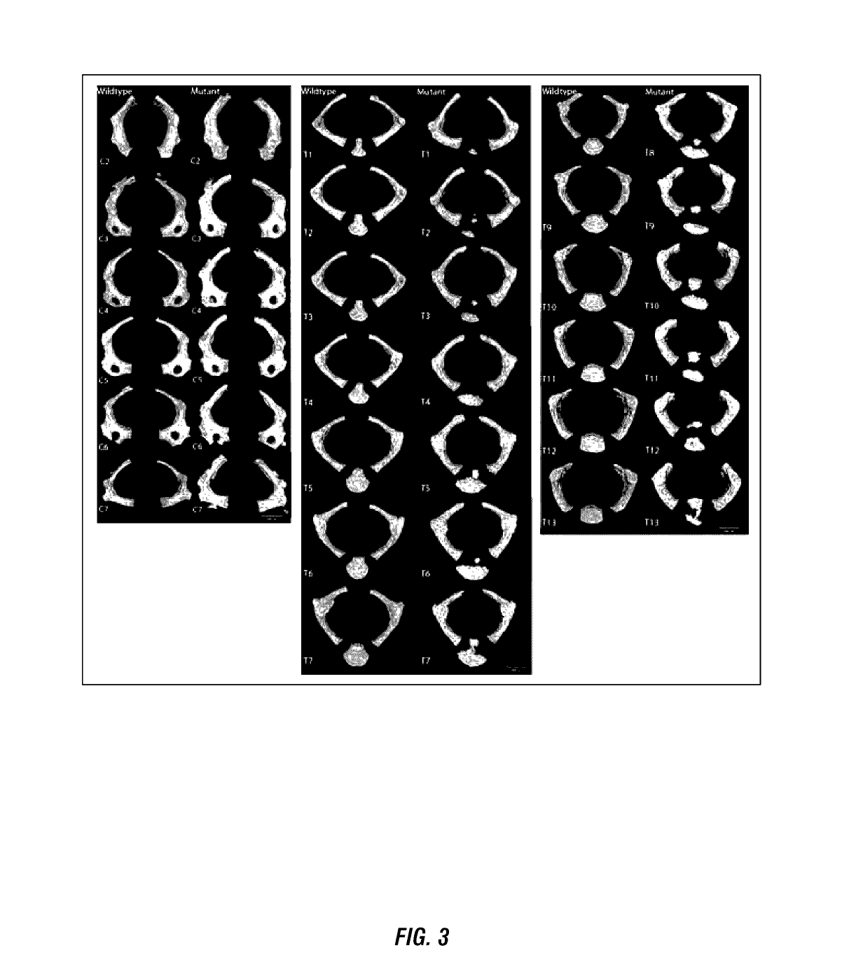

The gross morphology of each vertebra was compared among littermates. In the absence of Col 11A1, the vertebral arches exhibited a more rounded shape, in contrast to the ovoid shape of the vertebrae from control mice (FIG. 3). All vertebral bodies in the Col 11A1-deficient mice were reduced in size, appeared to have an altered shape and incomplete mineralization. Further, in contrast to WT, which exhibited a single mineralized component that comprised the vertebral body, multiple smaller mineralization foci and a lack of mineralization along the midline of the vertebral bodies was observed in the Col 11A1-deficient mice. The morphological changes in vertebral body formation were consistent with changes that lead to congenital spinal deformities which contribute to scoliosis and kyphosis.[46]

### 3.2. Bone Microarchitectural Parameters Dependent Upon the Expression of Col 11A1.

Quantitative changes to bone density of the vertebrae T1-T13 were identified in the Col 11A1-deficient mice compared to WT littermates. Microarchitectural parameters were determined for the thoracic vertebral arches and bodies, T1-T13 (Tables 1 and 2); indices describing trabecular thickness, (Tb.Th), trabecular number, (Tb.N), trabecular separation and trabecular percent bone volume were determined. In the vertebral arches, the trabecular thickness and percent bone volume were greater in the Col 11A1-deficient mice compared to WT littermates (31.7% and 32.8% increase, respectively). While trabecular spacing and number of Col 11A1-deficient mice showed differences when compared to WT, these differences were small and not statistically significant. Trabecular thickness and percent bone volume were greater in the vertebral bodies of the Col 11A1-deficient mice compared to WT littermates (80.4% and 67.2% increase, respectively) and trabecular spacing decreased in the Col 11A1-deficient mice compared to WT littermates (a decrease of 17%). As with the vertebral arches, a difference in trabecular number was observed, but the difference was not significant. A trend was observed in the percent bone volume of the vertebrae for both WT and Col 11A1-deficient mice and that was that the vertebrae, descending from anterior to posterior were less mineralized compared to the more anterior vertebrae.

### 3.2 Col 11A1-Dependent Changes in the Ribs.

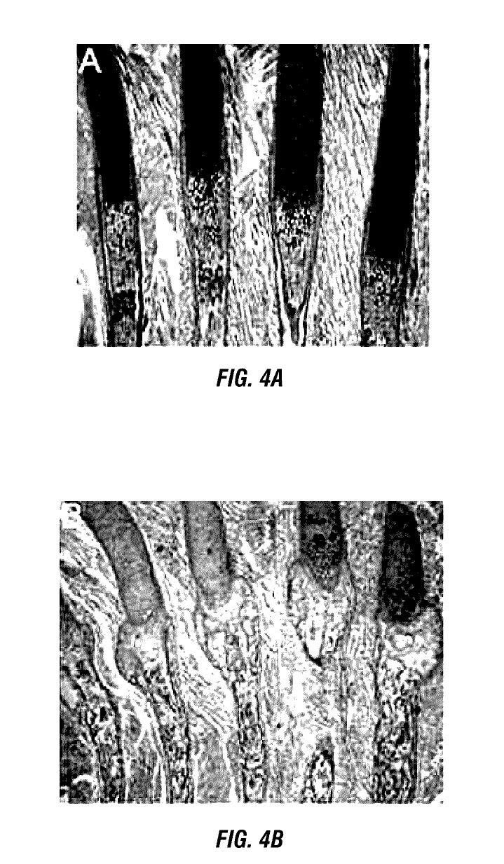

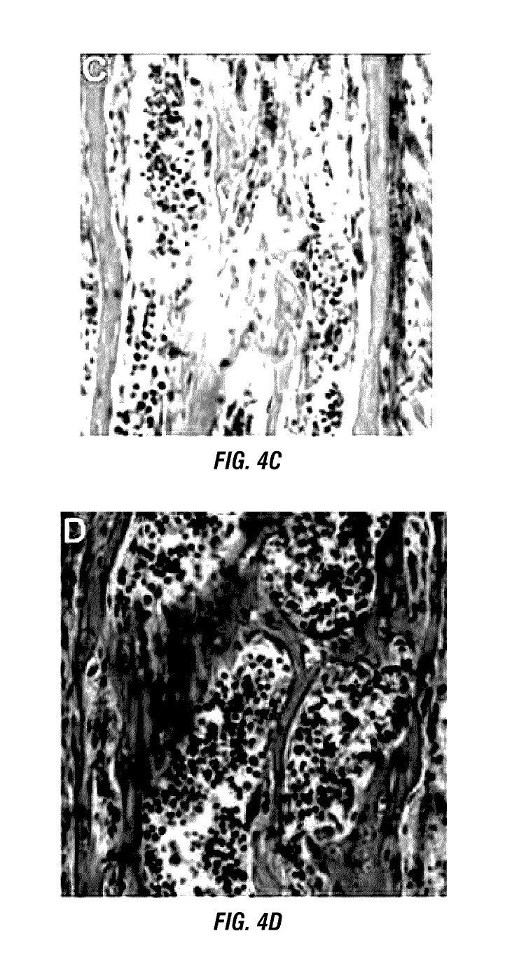

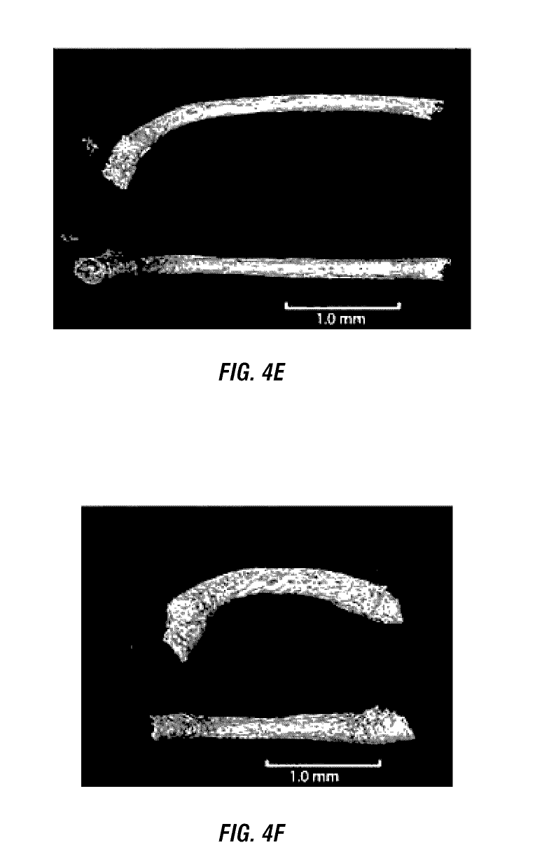

In the absence of Col 11A1, ribs developed a more severe curvature at the proximal end, near the point of attachment of the head and tubercle of the rib to the costal demifacet and transverse costal facet of the vertebrae respectively, apparent in FIG. 1 and FIG. 2. Histologic sections demonstrated an increase in mineralization and a more abrupt transition from growth plate cartilage to the mineralized zone, with excessive mineralized tissue in the ribs of Col 11A1-deficient mice (FIG. 4). The ribs of Col 11A1-deficient mice were shorter and thicker than WT controls. Overall, mineralization of ribs was more extensive in the Col 11A1-deficient mice compared to WT littermates (FIG. 4).

### 3.3. Histological Analysis of Embryonic Long Bone Formation.

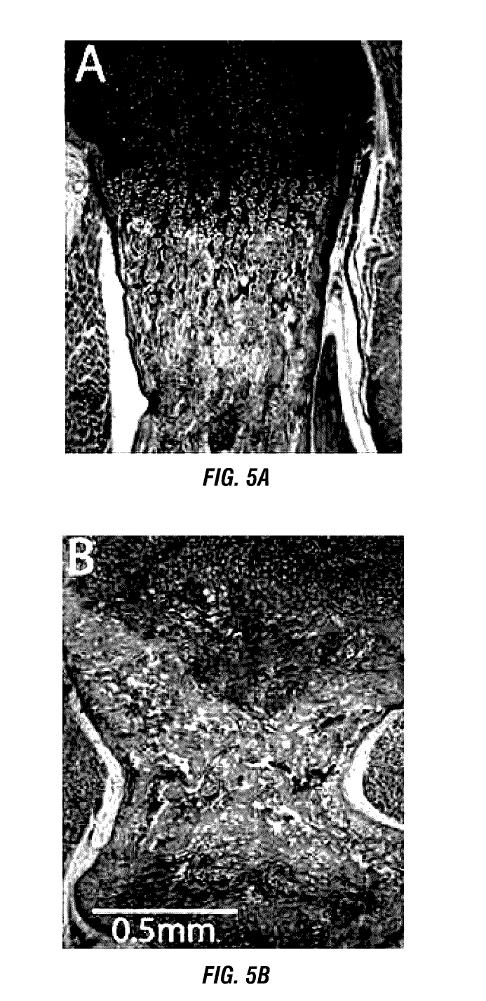

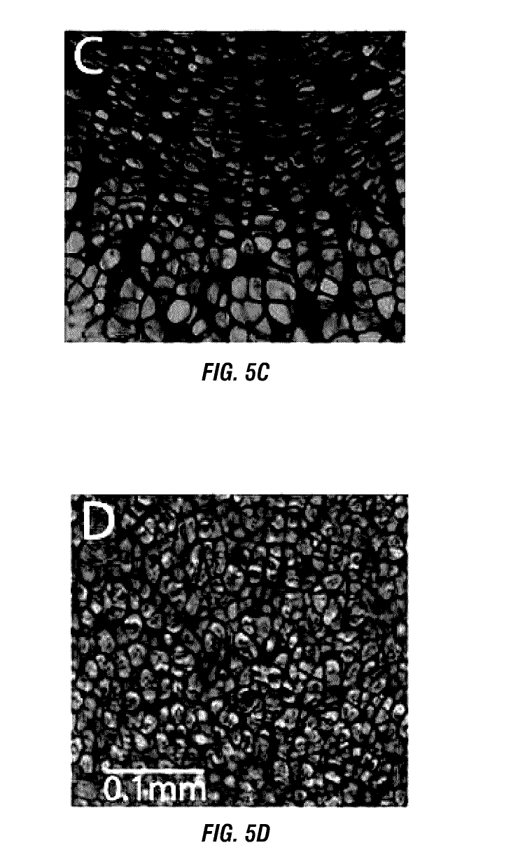

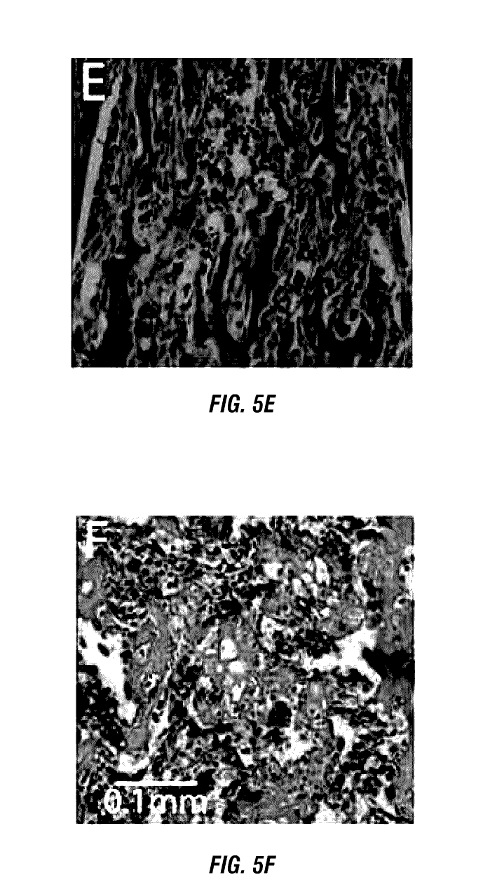

Trichrome stain was used to analyze mineralization in the long bones including femur, tibia, humerus, radius, and ulna of WT and Col 11A1-deficient mice. FIG. 5 demonstrates histological differences in the humerus. An increase in mineralized tissue was observed immediately adjacent to the lower hypertrophic zone of the growth plates. An increase in mineralized tissue was also observed at the periosteal surface of the newly formed bone collar, although the intensity of fast green staining for mineralized tissue was lower than that observed in the WT mice. Analysis of this data indicated a defect in perichondrial bone formation in the absence of Col 11A1.

### 3.4. Metaphyses, Diaphysis, and Cross-Sectional Area of the Col 11A1-Deficient Forelimbs.

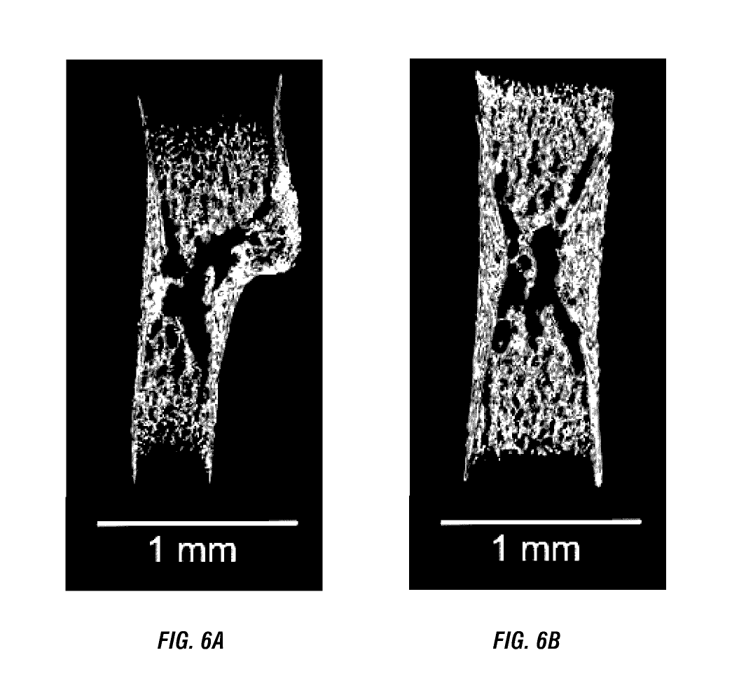

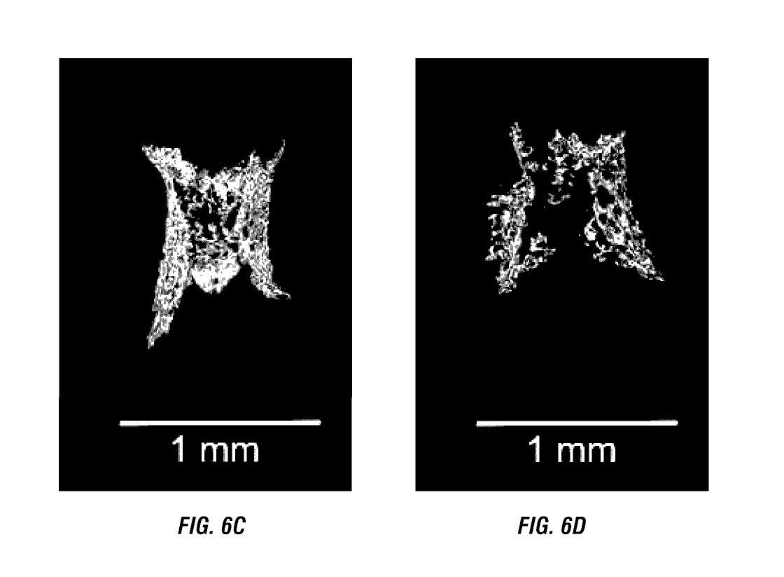

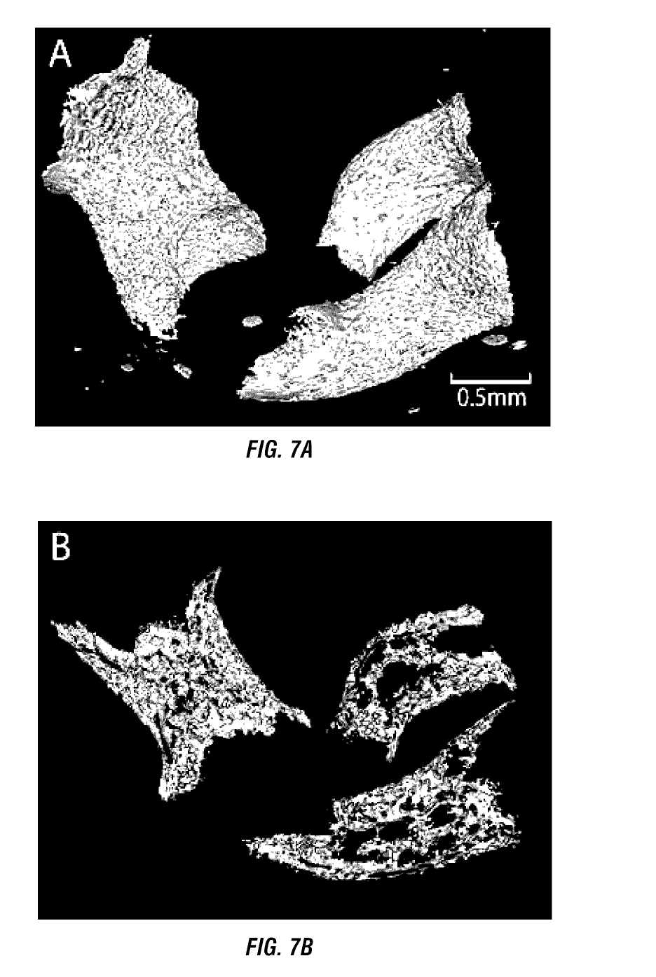

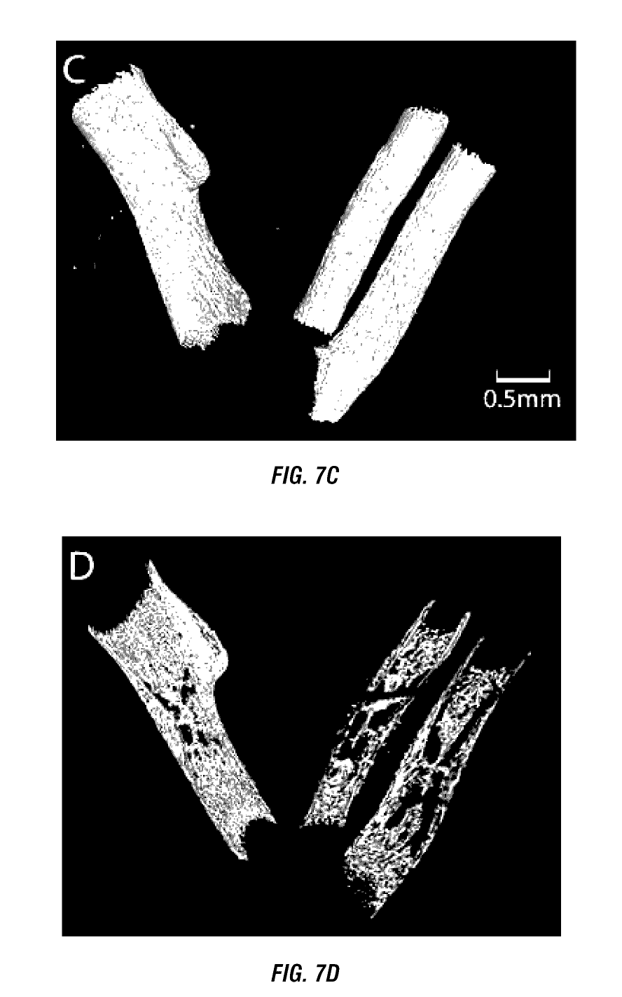

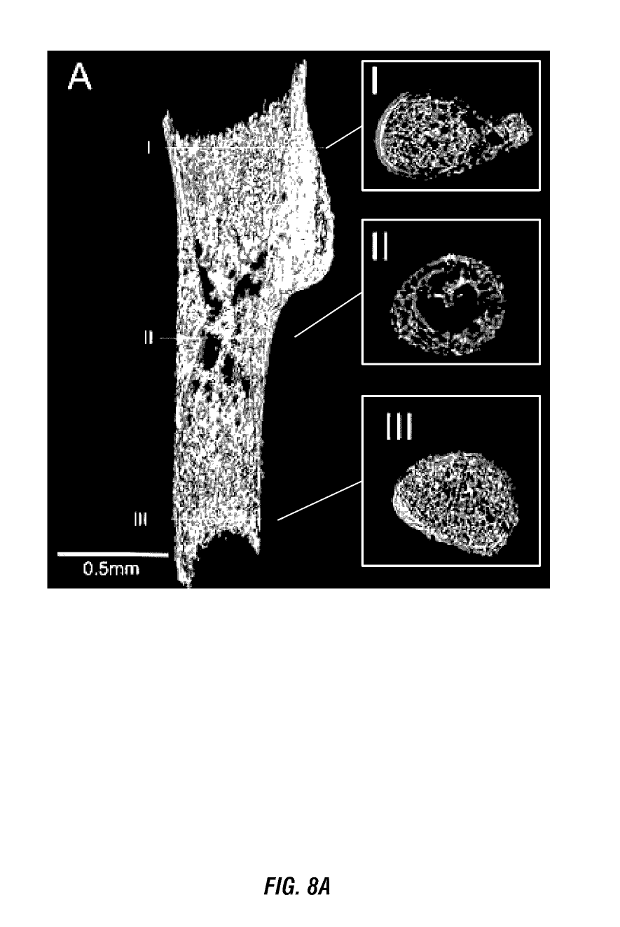

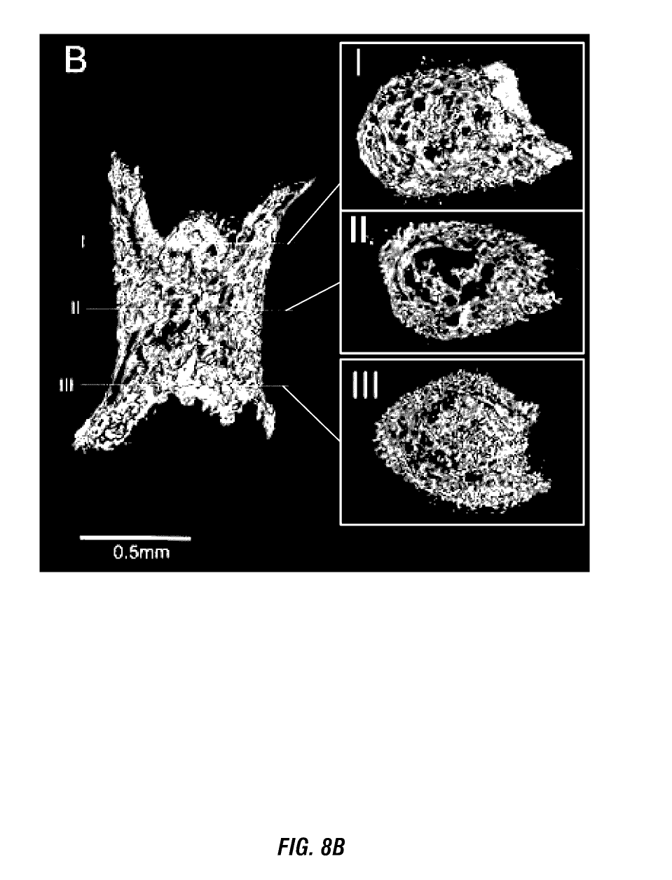

Col 11A1-deficient mice long bones were an average of 41% shorter than the WT humerus and femur (FIG. 6). The Col 11A1-deficient mice humeri exhibited an abnormally cylindrical shape atypical of a normal developing humerus, and lacked the deltoid tuberosity seen in the WT littermates (FIG. 7). The bones of the Col 11A1-deficient mice appeared wider at all points along the length of the bone (FIG. 8) and on average were 24% wider at the diaphysis, 15% wider at the proximal metaphysis, and 47% wider at the distal metaphysis (Table 3). Average cross-sectional area was found to be 80% greater at the diaphysis, 56% greater at the proximal metaphysis, and 26% greater at the distal metaphysis in the absence of Col 11A1. Interestingly, the Col 11A1-deficient long bones displayed an increase in mineralized tissue at the proximal metaphysis and a decrease of mineralized trabecular bone at the distal metaphysis.

Trabecular thickness, trabecular separation and trabecular percent bone volume were increased in the forelimb bones of the Col 11A1-deficient mice. Analysis of microarchitectural indices at the proximal metaphysis of the humerus showed differences in trabecular thickness (93% increase in Tb.Th), trabecular separation (17% increase in Tb.Sp), and trabecular percent bone volume (73% increase in BV/TV) in the absence of Col 11A1 expression. While consistently decreased in samples, the difference in trabecular number did not fall outside the 95% confidence interval for WT values (Table 4). No significant difference was detected for isotropy values or structure model index indicating similar relative prevalence of rods and plates in the three-dimensional structure of the trabecular bone for WT and Col 11A1-deficient mice (Table 4).

### 4. Discussion and Conclusions

Three-dimensional models were created from X-ray micro-CT images of skeletons from Col 11A1-deficient mice and these were compared to WT littermates. Relative to WT littermates, the percent bone volume was increased in the absence of Col 11A1 gene expression. Trabecular thickness and number were increased while trabecular separation was decreased in the Col 11A1-deficient mice. This study provides quantitative information on the microarchitecture of the skeleton and the role that Col 11A1 plays in bone development.

Differences in skeletal development were observed in the deltoid tuberosity of the humerus. The deltoid tuberosity was not formed in the absence of Col 11A1 expression. Periosteal bone thickness was greater in the absence of Col 11A1 expression compared to WT littermates, and this increase in bone thickness may be due to excessive appositional growth and mineralization within the periosteum, resulting in an increase in radial growth at the perichondrium relative to that of the control littermates. This finding may indicate a lack of regulation in bone collar formation in the absence of the Col 11A1 gene product and may indicate that Col 11A1 plays an essential role in the formation of the bone collar.

While the function of Col 11A1 is best characterized in the context of cartilage, Col 11A1 is also expressed in many other tissues, including bone. Recently, a role for Col 11A1 in osteoblast function was identified in a study in which osteoblast maturation was accelerated in the absence of specific Col 11A1 isoforms and inhibited in the presence of a recombinant fragment of Col 11 A1.[47] Thus, recent findings indicate a direct role in osteoblast function and differentiation which is distinct from the previously reported role in the assembly of the extracellular matrix synthesized by chondrocytes.

Phenotypic overlap between the Col 11A1 mutation and that of other structural molecules of ECM may indicate a shared function or a direct molecular interaction between the two constituents within the matrix. Candidate molecules for which a phenotypic overlap with Col 11A1 exists include Col2a1, link protein, chondroitin sulfate sulfotransferase 1, PTHrP, Indian hedgehog, and FGFRs.[48-52] Mice overexpressing BMP4 in cartilage have widened bones containing thick trabeculae, possibly because of expansion of cartilage anlagen.[53] Thickened trabeculae were also observed in a Col 11a2-BMP4 transgenic mouse at 18.5dpc. In the Col 11a2-BMP4 mouse, the epiphyseal cartilages of the humeri were widened compared to WT. Additionally, the diaphyses undergoing mineralization were also widened, accompanied by the observation of thickened trabecular bone in the marrow cavities. When Noggin expression was placed under the control of the Col 11a1 promoter in transgenic mice, micro-CT analysis revealed a greater volume of trabecular bone during embryonic stage 17.5 dpc to 3 weeks after birth, when compared to WT.[53]

It is possible that the changes in bone microarchitecture observed in the absence of the Col 11A1 gene product may be explained by primary changes to the structure of the cartilage anlagen during endochondral ossification, leading to subsequent changes in bone microarchitecture secondarily. A wider cartilaginous anlagen may result in the production of a widened bone structure. Additionally, altered properties of the cartilaginous anlagen due to the absence of Col 11A1 may result in changes to distribution and delivery of cell signaling molecules that control bone growth and the spatial and temporal control of bone mineralization. Future studies are needed to focus on potential mechanisms of Col 11A1's effect on mineralization, directly and indirectly.

Mutations in the genes encoding collagen type XI alpha chains result in a number of spondylo-epiphyseal dysplasias.[48] Among these conditions, are the human chondrodysplasias, Stickler syndrome, Marshall syndrome, Wagner syndrome, and Fibrochondrogenesis.[49,55,56] Collagen type XI-related syndromes present a number of clinical skeletal symptoms, including abnormal epiphyseal development, irregularity of the margins of the vertebral bodies, thick calvaria, short stature, and intracranial calcifications (OMIM: 154780, 108300, 143200).

Overall, the changes observed in this study suggest that the absence of Col 11A1 gene expression in developing bone resulted in thickened trabecular bone and reduction in endosteal bone turnover, contributing to alterations in marrow cavity formation and an increase in periosteal bone apposition leading to a defect in primary spongiosa formation and a thicker bone collar. These data suggest that Col 11A1 may be a regulator of osteogenesis and mineralization of the skeleton during endochondral ossification. The changes to the bone collar observed in these studies suggest a role for Col 11A1 in intramembranous bone formation. Future investigations from our laboratory will focus on determining the molecular mechanism of Col 11A1 involvement in chondrogenic and osteoblastic differentiation during endochondral and intramembranous ossification.

The impact of a Col 11A1-deficiency on the formation of vertebral bodies was an unexpected result. A review of the literature indicated that hemivertebrae formation can be associated with two different types of defects, one that occurs during the prechondral stage of vertebral body formation and one that occurs at the ossification stage. It is interesting to note that Col 11A1 mutations have been identified by genome-wide association studies for lower back pain and lumbar disc degeneration in some populations.[57]

### REFERENCES

[1] Jacenko O, Roberts D W, Campbell M R, McManus P M, Gress C J, Tao Z. Linking hematopoiesis to endochondral skeletogenesis through analysis of mice transgenic for collagen X. Am J Pathol. 2002; 160:2019-34.
[2] Horton W A. The biology of bone growth. Growth Genet Horm. 1990; 6: 21-3.
[3] Kronenberg H M. Developmental regulation of the growth plate. Nature. 2003; 423:332-6.
[4] Ornitz D M, Marie P J. FGF signaling pathways in endochondral and intramembranous bone development and human genetic disease. Genes Dev. 2002; 16:1446-65.
[5] Olsen B R, Reginate A M, Wang W. Bone development. Annual Rev Cell Dev Biol. 2000; 16:191-220.
[6] Bruder S P, Caplan A I. Cellular and molecular events during embryonic bone development. Connect Tissue Res. 1989; 20:65-71.
[7] Hatori M, Klatte K J, Teixeira C C, Shapiro I M. End labeling studies of fragmented DNA in the avian growth plate: evidence of apoptosis in terminally differentiated chondrocytes. J Bone Miner Res. 1995; 10:1960-8.
[8] Kahn, A J, Simmons, D J. Investigation of cell lineage in bone using a chimera of chick and quail embryonic tissue. Nature. 1975; 258:258-325.
[9] Manolagas S C, Jilka R L. Bone marrow, cytokines, and bone remodelling. Emerging insights into the pathophysiology of osteoporosis. New Engl J Med. 1995; 332:305-11.
[10] Ash, P, Loutit, J F, Townsend, K M S. Osteoclasts derived from haemoatopoietic stem cells. Nature. 1980; 283:669-70.
[11] Blair, H C, Kahn, A J, Crouch, E C, Jeffrey, J J, Teitelbaum, S L. Isolated osteoclasts resorb the organic and inorganic components of bone. J Cell Biol. 1986; 102(4): 1164-72.
[12] Van der Rest M, Garrone R. Collagen family of proteins. FASEB J. 1991; 5:2814-23.
[13] Holmes D F, Kadler K E. The 10+4 microfibril structure of thin cartilage fibrils. PNAS. 2006; 103:17429-254.
[14] Blaschke U K, Eikenberry E F, Hulmes D J, Galla H J, Bruckner P. Collagen XI nucleates self-assembly and limits lateral growth of cartilage fibrils. J Biol Chem. 2000; 275:10370-8.
[15] Gregory K E, Oxford J T, Chen, Y, et al. Structural organization of distinct domains within the non-collagenous N-terminal region of collagen type XI. J Biol Chem. 2000; 275:11498-506.
[16] Morris N P, Bachinger H P. Type XI collagen is a heterotrimers with the composition (1 alpha, 2 alpha, 3 alpha) retaining non-triple-helical domains. J Biol Chem. 1987; 262:11345-50.
[17] Fisher H, Stenling R, Rubio C, Lindblom A. Colorectal carcinogenesis is associated with stromal expression of Col 11A1 and COL5A2. Carcinogenesis. 2001; 22:875-78.
[18] Niyibizi C, Eyre D R. Identification of the cartilage α1(XI) chain in type V collagen form bovine bone. FEBS Lett. 1989; 242:314-18.
[19] Mayne R, Brewton R G, Mayne P M, Baker J R. Isolation and characterization of the chains of type V/type XI collagen present in bovine vitreous. J Biol Chem. 1993; 268:9381-6.
[20] Sandberg M M, Hirvonen H E, Elima K J M, Vuorio E L Co-expression of collagens II and XI and alternative splicing of exon 2 of collagen II in several developing human tissues. Biochem J. 1993; 294:595-602.
[21] Brown K E, Lawrence R, Sonenshein G E. Concerted modulation of α1(XI) and α2(V) collagen mRNAs in bovine vascular smooth muscle cells. J Biol Chem. 1991; 266:23268-73.
[22] Yamazaki M, Majeska R J, Yoshioka H, Moriya H, Einhorn T A. Spatial and temporal expression of fibril-forming minor collagen genes (types V and XI) during fracture healing. J Orthop Res. 1997; 15:757-64.
[23] Yoshioka H, Ramirez F. Pro-α1 (XI) collagen Structure of the amino-terminal propeptide and expression of the gene in tumor cell lines. J Biol Chem. 1990; 265:6423-6.
[24] Kleman J P, Hartmann D J, Ramirez F, van der Rest M. The human rhabdomyosarcoma cell line A204 lays down a highly insoluble matrix composed mainly of α1 type-XI and α2 type-V collagen chains. Eur J Biochem. 1992; 210:329-35.
[25] Medeck R J, Sosa S, Morris N P, Oxford J T. BMP-1-mediated proteolytic processing of alternatively spliced isoforms of collagen type XI. Biochem J. 2003; 376:361-8.
[26] Davies G B M, Oxford J R T, Hausafus L C, Smoody B F, Morris N P. Temporal and spatial expression of spliceforms of the α1(XI) collagen gene in fetal rat cartilage. Dev Dynam. 1998; 213:13-26.
[27] Zhidkova N I, Justice S K, Mayne R. Alternative mRNA processing occurs in the variable region of the pro-alpha 1(XI) and pro-alpha 2(XI) collagen chains. J Biol Chem. 1995; 270:9486-93.
[28] Oxford J T, Doege K J, Morris N P. Alternative exon splicing within the amino terminal-nonhelical domain of the rat pro α1(XI) collagen chain generates multiple forms of the mRNA transcript which exhibit tissue dependent variation. J Biol Chem. 1995; 270:9478-85.
[29] Ryan M C, Sandell L J. Differential expression of a cysteine-rich domain in the amino-terminal propeptide of Type II (cartilage) procollagen by alternative splicing of mRNA. J Biol Chem. 1990; 265:10334-9.
[30] Morris N P, Oxford J T, Davies G B, Smoody B F, Keene D R. Developmentally regulated alternative splicing of the α1(XI) collagen chain: spatial and temporal segregation of isoforms in the cartilage of fetal rat long bones. J Histochem Cytochem. 2000; 48:725-41.
[31] Li Y, Lacerda D A, Warman M L, et al. A fibrillar collagen gene Col 11A1 is essential for skeletal morphogenesis. Cell. 1995; 80:423-30.
[32] Seegmiller R E, Fraser F C, Sheldon H. A new chondrodystrophic mutant in mice. J Cell Biol. 1971; 48:580-93.
[33] Fernandes R J, Weis M, Scott M A, Seegmiller R E, Eyre D R. Collagen XI chain misassembly in cartilage of the chondrodysplasia (cho) mouse. Matrix Biol. 2007; 26:597-603.
[34] Seegmiller R E, Cooper C A, Houghton M J, Carey J C. Pulmonary hypoplasia in chondrodystrophic mice. Teratology. 1986; 33:339-47.
[35] Hepworth W B, Seegmiller R E, Carey J C. Thoracic volume reduction as a mechanism for pulmonary hypoplasia in chondrodystrophic mice. Pediatr Pathol. 1990; 10:919-29.
[36] Monson C B, Seegmiller R E. Ultrastructural studies of cartilage matrix in mice homozygous for chondrodysplasia. J Bone Joint Surg. [Am] 1981; 63:637-44.
[37] Seegmiller R E, Myers R A, Dorfman A, Horwitz A L. Structure and associative properties of cartilage matrix constituents in mice with hereditary chondrodysplasia. Connect Tissue Res. 1981; 9:69-77.
[38] Szymko-Bennett Y M, Kurima K, Olsen B, Seegmiller R, Griffith A J. Auditory function associated with Col 11A1 haploinsufficiency in chondrodysplasia (cho) mice. Hearing Research. 2003; 175:178-82.
[39] Stephens T D, Seegmiller R E. Normal production of cartilage glycosaminoglycan in mice homozygous for the chondrodysplasia gene. Teratology. 1976; 13:317-26.
[40] Parfitt A M, Drezner M K, Glorieux F H, et al. Bone Histomorphometry: standardization of nomenclature, symbols and units. J Bone Miner Res. 1987; 2:595-610.
[41] Ulrich D, van Rietbergen B, Laib A, Rüegsegger P. The ability of three-dimensional structural indices to reflect mechanical aspects of trabecular bone. Bone. 1999; 25:55-60.
[42] Hildebrand T, Ruegsegger P. A new method for the model independent assessment of thickness in three dimensional images. J Microsc. 1997; 185:67-75.
[43] Lorensen W E, Cline H E. Marching cubes: a high resolution 3d surface construction algorithm. Computer Graphic.s 1987; 21:163-9.
[44] Carson, F L, Hladak C. Histotechnology: A Self-Instructed Text. 3rd ed. Hong Kong: American Society for Clinical Pathology Press. 2009
[45] Gomori, G. A rapid one-step trichrome stain. Am.J.Clin.Path. 1950; 20; 661-4.
[46] Goldstein I, Makhoul I R, Weissman A, Drugan A. Hemivertebra: Prenatal diagnosis, incidence and characteristics. Fetal Diag and Therapy. 2005; 20:121-6.
[47] Kahler R A, Yingst S M, Hoeppner L H, et al. Collagen 11a1 is indirectly activated by lymphocyte enhancer-binding factor 1(lef1) and negatively regulated osteoblast maturation. Matrix Biology. 2008; 27(4):330-8.
[48] Spranger J. The type XI collagenopathies. Pediatr Radiol. 1998; 28:745-50.
[49] Watanabe H, Yamada Y. Chondrodysplasia of gene knockout mice for aggrecan and link protein. Glycoconjugate J. 2003; 19:269-73.
[50] Klüppel M, Wight T N, Chan C, Hinek A, Wrana J L. Maintenance of chondroitin sulfonation balance by Chondroitin-4-sulfotransferase-1 is required for chondrocyte development and growth factor signaling during cartilage morphogenesis. Development. 2005; 132:3989-4003.
[51] Razzaque M S, Soegiarto D W, Chang D, Long F, Lanske B. Conditional deletion of Indian hedgehog from collagen type 2alpha1-expressing cells results in abnormal endochondral bone formation. J Pathology. 2005; 207:453-61.
[52] Delezoide A L, Benoist-Lasselin C, Legeai-Mallet L, Le Merrer M, Munnich A, Vekemans M, Bonaventure J. Spatio-temoral expression of FGFR 1, 2, 3 genes during human embryo-fetal ossification. Mech Dev. 1998; 77:19-30.
[53] Okamoto M, Murai J, Yoshikawa H, Tsumaki N. Bone morphogenetic proteins in bone stimulate osteoclasts and osteoblasts during bone development. J Bone Mineral Res. 2006; 21:1022-33.
[54] Seegmiller, R E, Monson, C B. Scanning electron microscopy of cartilage in mice with hereditary chondrodysplasia. SEM. 1982; 111:1259-67.
[55] Tompson S W, Bacino C A, Safina, N P, et al. Fibrochondrogenesis Results from Mutations in the Col 11A1 Type XI Collagen Gene. Am J Human Genetics. 2010; 87:708-12.
[56] Akawi N A, Al-Gazali L, Ali B R. Clinical and molecular analysis of UAE fibrochondrogenesis patients expands the phenotype and reveals two Col 11A1 homozygous null mutations. Clin Genet. 2012; 82:147-56.
[57] MioF, Kazuhiro C, Yuichiro H, et al. A functional polymorphism in Col 11A1, which encodes the alpha 1 chain of type XI collagen, is associated with susceptibility to lumbar disc degeneration. Am. J. Hum Genet. 2007; 81(6):1271-7.

TABLE 1
Densitometric indices for the vertebral arches (mean % difference ±
SD) between WTand Col 11A1^{-/-}

| | BV/TV (%) | Tb. Th (μm) | Tb. N (1/mm) | Tb. Sp (μm) |
|---|---|---|---|---|
| % difference | 31.7 | 32.8 | −1.01 | 0.03 |
| SD | 3.9 | 3.4 | 1 | 1 |
| | p < 0.05 | p < 0.05 | ns | Ns |

BV/TV, Tb. Th, Tb. N, and Tb. Sp are reported as percent difference between Col 11A1-deficient mice compared to WT littermates, reported as mean with SD. Statistical differences are reported as p values unless determined to be not significant (ns). Control mice (n = 6), Col 11A1-deficient mice (n = 3).

TABLE 2
Densitometric indices for the vertebral bodies (mean % difference ±
SD) between WT and Col 11A1^{-/-}

| | BV/TV (%) | Tb. Th (μm) | Tb. N (1/mm) | Tb. Sp (μm) |
|---|---|---|---|---|
| % difference | 80.4 | 67.2 | 8.26 | −17 |
| SD | 8.5 | 7.9 | 1 | 2 |
| | p < 0.05 | p < 0.05 | ns | p < 0.05 |

BV/TV, Tb. Th, Tb. N and Tb. Sp are reported as percent difference between Col 11A1-deficient mice compared to WT littermates, reported as mean with SD. Statistical differences are reported as p values unless determined to be not significant (ns). Control mice (n = 6), Col 11A1-deficient mice (n = 3).

TABLE 3
Structural indices for humeri are reported as mean +
SD for WT and Col 11A1^{-/-}

| | | Proximal | | Distal |
|---|---|---|---|---|
| | | Metaphysis | Diaphysis | Metaphysis |
| | Length | diameter | diameter | diameter |
| Genotype | (μm) | (μm) | (μm) | (μm) |
| WT | 2409 ± 33.6 | 806 + 16.9 | 592 ± 11.6 | 528 ± 15.2 |
| Col 11A1^{-/-} | 1422 ± 65.5 | 930 + 38.4 | 735 ± 37.4 | 778 ± 86.1 |
| % difference | 41.0 | 15.4 | 24.2 | 47.4 |
| | p < 0.0001 | p < 0.05 | p < 0.05 | p < 0.05 |

Values are reported as mean ± SD. Statistical differences are reported as p values unless determined to be not significant (ns). Control mice (n = 6), Col 11A1-deficient mice (n = 3).

TABLE 4
Densitometric indices for the humeri (mean ±
SD) WT and Col 11A1^{-/-}

| | BV/TV | | | Tb. Th | Tb. N | Tb. Sp |
|---|---|---|---|---|---|---|
| Genotype | (%) | SMI | DA | (μm) | (1/mm) | (μm) |
| WT | 25.3 ± | 2.1 ± | 0.90 ± | 18.32 ± | 13.8 ± | 33.2 ± |
| | 2.9 | 0.17 | 0.025 | 0.22 | 1.5 | 1.81 |
| Col | 43.7 ± | 1.87 ± | 0.84 ± | 35.3 ± | 12.4 ± | 38.9 ± |
| 11A1^{-/-} | 4.1 | 0.033 | 0.082 | 3.7 | 0.81 | 1.19 |
| % differ- | 72.7 | 11.0 | 0.1 | 91.6 | 10.2 | 17.2 |
| ence | p < | ns | ns | p < | ns | p < |
| | 0.05 | | | 0.05 | | 0.05 |

BV/TV, Tb. Th, Tb. N, and Tb. Sp, are reported as mean ± SD. Statistical differences are reported as p values unless determined to be not significant (ns). Isotropy values (DA) range from 0 (total isotropy) to 1 (total anisotropy). Structure model index (SMI) indicating relative prevalence of rods and plates in 3 dimensional structure range from 0 (plate-like) to 3 (rod-like). Control mice (n = 6), Col 11A1-deficient mice (n = 3).

### Example 2

### Alternatively Spliced Isoforms of Col 11A1 Regulate Osteoblast Differentiation Through the BMP-2 Signaling Pathway

### Introduction

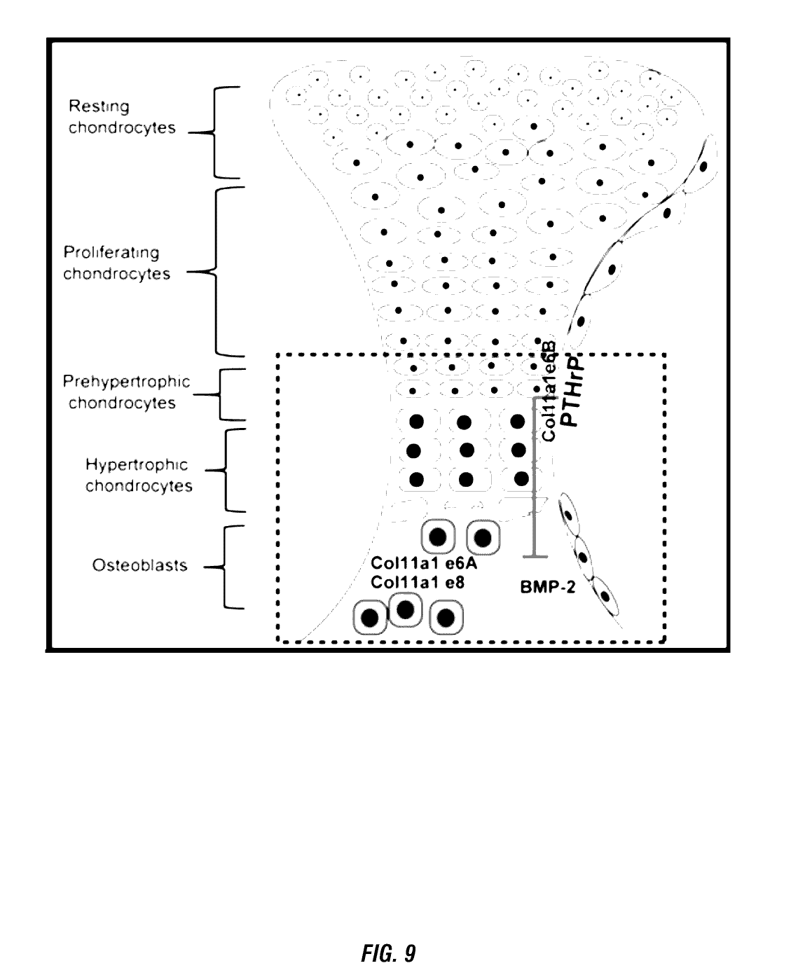

The skeleton develops via processes of endochondral and intramembranous ossification. Long bones elongate by means of interstitial growth and widen via appositional growth. Interstitial growth of long bones occurs via endochondral ossification in which cartilage is replaced by bone; whereas appositional growth occurs as progenitor cells of the periosteum differentiate into osteoblasts and deposit bone. The process of bone development starts during fetal life and persists until puberty when growth ceases. In a typical long bone, endochondral ossification occurs at the growth plate, which consists of specific zones (FIG. 9) (1).

The resting zone contains pre-chondrocytes that differentiate into mature chondrocytes forming the proliferative zone. Proliferating chondrocytes then align into columns, terminally differentiate into hypertrophic chondrocytes, and undergo apoptosis. As hypertrophic chondrocytes undergo apoptosis, pre-osteoblast progenitor cells of the periosteum differentiate into osteoblasts, the newly formed bone tissue is innervated, blood vessels infiltrate, and calcification occurs. The highly dynamic environment of the growth plate involves a complex network of hormones, paracrine molecules, extracellular matrix molecules, and growth factors that work together to facilitate processes of cell proliferation and differentiation that lead to proper tissue development.

Collagens are abundant extracellular matrix proteins that maintain tissue structure and control the environment in which cells find themselves (2). Mutations in collagen XI have been linked with rare and detrimental human diseases including autosomal dominant Marshall's and Stickler syndromes, the more severe autosomal-recessive fibrochondrogenesis, and otospondylomegaepiphyseal dysplasia (OSMED) (3-8). In developing cartilage, mature collagen XI protein consists of three different alpha chains—Col 11A1, Col 11a2, and Col 11a3 (an over-glycosylated form of Col 2a1). Later in development, Col5a1 can replace the Col 11a2 chain in articular cartilage (9). However, in bone, a collagen V/XI hybrid molecule constitutes the minor fibrillar collagen, consisting of Col5a1, Col5a2, and Col 11A1 (10, 11). Targeted mutations of Col 11A1 in mice closely reflect characteristics of the human diseases. Mice lacking Col 11A1 die neonatally and exhibit a phenotype that includes facial dysmorphism and wider but shorter metaphyses in the long bones, suggesting a role for Col 11A1 in proper growth plate development (12-14). While a direct effect on growth plate cartilage has been defined, the consequences of absent or reduced levels of Col 11A1 expressed by osteoblasts have not been fully investigated. Col 11a2 null mice also exhibit a cartilage phenotype, but it is milder and similar to OSMED patients (15, 16).

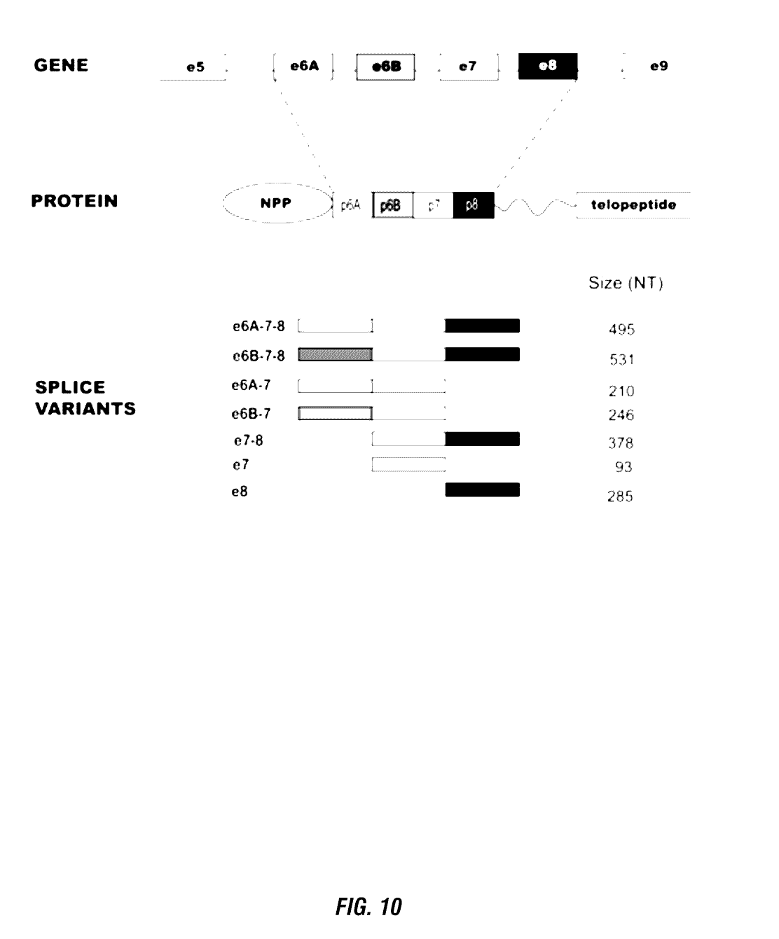

The Col 11A1 alpha chain of collagen XI contains a non-collagenous amino terminal domain (NTD) composed of an amino propeptide (Npp) and a variable region (VR) (FIG. 10) (17, 18). The NTD is found on the surface of heterotypic collagen fibrils and is thought to sterically hinder further addition of collagen molecules, thus regulating fibril diameter (19, 20). Interestingly, alpha chains of collagen XI undergo alternative splicing. Col 11a2 splice variants quickly converge to produce a single splice isoform during development (21). Conversely, the variable region of Col 11A1 undergoes alternative splicing in a spatiotemporal manner and the different splice forms persist as mature proteins in the ECM, suggesting a role in maintaining or directing cellular and matrix events in addition to mediating fibrillogenesis, perhaps by interaction with other extracellular matrix molecules (22-25) and cells.

Alternative splicing of exons 6A, 6B, 8, and most recently 7 within the variable region of Col 11A1 can produce more than eight different isoforms that show distinct spatiotemporal expression patterns in the growth plate (FIG. 10) (17, 24, 26). In chondrocytes, the timing of Col 11A1 splicing correlates well with Col2a1 splicing at the onset of chondrogenesis (22). Collagen type II exon 2 is retained in prechondrocytes forming the longer IIA isoform; however as cells undergo differentiation and become mature chondrocytes, exon 2 is spliced out, forming the shorter IIB isoform. Alternative splicing of collagen type II is regulated by bone morphogenetic protein-2 (BMP-2) during chondrogenesis (27, 28). However, a role for BMP-2 in Col 11A1 alternative splicing has not been described.

BMPs were first identified from demineralized bone matrix and are able to induce ectopic bone formation (29, 30). BMP-2 specifically plays a key role in the development of cartilage and bone in many species (31). In the growth plate, BMP-2 promotes chondrocyte hypertrophy in part by inducing collagen type X expression and is also important in periosteum-mediated bone formation (32-35). The periosteal bone collar serves as a reservoir for progenitor cells that are capable of differentiating into chondrocytes as well as osteoblasts, depending on the local cues from the environment (36). In vitro, mouse C2C12 cells have been used as a model for cells of the periosteum due to their myo-chondro-osteogenic potential (37-39). Further, C2C12 cells are commonly used to study the mechanisms underlying BMP-2 mediated osteoblast differentiation (38, 40, 41). C2C12 cells treated with BMP-2 readily differentiate into osteoblasts and express osteoblast markers including Runx2, OCN, Col1a1, and ALP. In the canonical pathway, BMP-2 binds to its receptor BMPRII, which then dimerizes with and phosphorylates BMPRI. Activated BMPRI phosphorylates SMAD 1/5/8 proteins, which together with co-SMAD 4 enter the nucleus and regulate the transcription of target genes (42). BMP-2-induced expression of osteoblast markers Runx2 and Osterix in C2C12 cells can be partially prevented by parathyroid hormone related peptide (PTHrP) (40). Further, BMP-2 treatment of C2C12 cells downregulates PTHrP expression in these cells, while upregulating PTH1R, a marker for osteoblast differentiation (40, 43). PTHrP is secreted by perichondrial and particular cells and binds to its receptor PTH1R expressed by proliferative chondrocytes to maintain chondrocytes in a proliferative state and suppress their terminal differentiation to hypertrophy (44). The most well-known mechanism by which PTHrP exerts its functions in the growth plate is via a negative feedback loop with Indian Hedgehog (45). Although PTHrP has a well-defined role in chondrocytes of the growth plate, its role in osteoblast proliferation and differentiation is disputed. Osteoblasts express high levels of PTH1R, and PTHrP haploinsufficient mice exhibit osteopenia suggesting a role for PTHrP signaling in bone formation (46, 47). Currently, it is known that intermittent PTHrP exposure increases bone mass, while continuous exposure exerts the opposite effect and decreases bone mass (48-51). The effects of PTHrP on osteoblast differentiation have been controversial with several studies showing pro-osteogenic effects (52-54), while others have shown anti-osteogenic effects (55-58). The opposing effects of PTHrP on osteoblast differentiation have been attributed to differences among cell types, culture condition, dosage, and method of delivery.

We have previously reported that a cartilage specific Col 11A1 recombinant protein fragment containing the region encoded by exon 6B inhibited ALP expression in C2C12 and MC3T3 osteoblasts (59). Considering the results from previous studies, we hypothesized that Col 11A1 would affect periosteal bone architecture and further, that Col 11A1 could regulate osteoblast differentiation in a BMP-dependent manner. Interestingly, more recent studies have shown a significant increase in overall Col 11A1 expression during osteoblast differentiation (60-62). In addition, recent genome-wide association studies (GWAS) have suggested that Col 11A1 acts in the growth plate to regulate height (63). Such findings led us to ask whether the expression and alternative splicing of Col 11A1 is regulated by PTHrP and BMP-2 during osteoblast differentiation and whether Col 11A1 regulates activity of BMP-2.

Here, we investigated a role for BMP-2 and PTHrP in Col 11A1 expression and alternative splicing in pluripotent mesenchymal C2C12 cells. Further, we studied the effects of both Col 11A1 knockdown and the addition of exogenous recombinant fragments of Col 11A1 on BMP-2 mediated osteoblast differentiation. Our results demonstrated that BMP-2 treatment increased Col 11A1 expression levels in C2C12 cells and led to the inclusion of exons 6A, 7, and 8, while PTHrP alone did not affect Col 11A1 expression. However, PTHrP diminished BMP-2-induced changes in the expression levels of specific exons of Col 11A1. Col 11A1 knockdown was found to initially reduce BMP-2-stimulated changes in osteoblast marker expression during the first 24 hours, but later, correlated to an increase osteoblast marker expression. The addition of Col 11A1[p6B-7] NTD protein fragment inhibited BMP-2-induced expression of osteoblast markers ALP and Col 1a1. Addition of the Col 11A1 [p7-8] NTD fragment significantly promoted BMP-2-induced expression of osteoblast markers OCN and Col 1a1. Overall, our study introduces Col 11A1 as a novel marker for osteoblast differentiation that functions in a splice form-specific manner, thus coupling endochondral ossification to bone collar formation and osteoblasts differentiation.

Researchers have investigated the mechanism of growth plate maturation for decades, however, the molecular interactions between growth factors, the extracellular matrix, and the resident cells are not fully understood. An understanding is essential if we are to be able to devise treatments for regeneration and repair of complex structures such as the growth plate. Here, we demonstrate the role of BMP-2 in the regulation of Col 11A1 alternative splicing and expression, and in a reciprocal manner, the role of Col 11A1 in regulating BMP-2 activity during osteoblast differentiation in a splice variant-specific manner. Col 11A1 possesses the characteristics of a coupling factor for the coordination of endochondral ossification with the formation of the bone collar of developing bones.

### Materials and Methods

### Micro-CT Analysis

Wildtype and Col 11A1^{-/-} littermates at embryonic day 17.5 were scanned with a SkyScan 1172 high-resolution micro-CT scanner (MicroPhotonics, Aartselaar, Belgium) to generate data sets with a 1.7 μm³ isotropic voxel size using an acquisition protocol that consisted of X-ray tub settings of 60 kV and 250 μA, exposure time of 0.147 seconds, six-frame averaging, a rotation step of 0.300 degrees, and associated scan times were approximately 7 hours. Following scanning, two-dimensional reconstructions were used to produce 6000 serial 4000×4000 pixel cross-sectional images.

### Cell Culture and Differentiation

The mouse pluripotent cell line, C2C12 was purchased from ATCC and maintained in DMEM (Sigma Chem. Co., St. Louis, Mo.) supplemented with 10% fetal bovine serum (FBS) (GIBCO, Grand Island, N.Y.) and antibiotics (100 units/mL of penicillin-G and 100 μg/mL streptomycin). Cells were kept at 37° C. in a humidified atmosphere of 5% CO₂ in air. To differentiate C2C12 cells into osteoblasts, cells were plated at 2×10⁴ cells/cm² in DMEM supplemented with 5% FBS, 100 U/mL penicillin and 100 μg/mL streptomycin and recombinant human BMP-2 (300 ng/mL) (R&D Systems, Minneapolis, Minn.). Control cells were kept in DMEM supplemented with 5% FBS and antibiotics. Medium was changed every 72 hours and supplemented with fresh BMP-2. For PTHrP experiments, cells were treated with BMP-2 for five days after which PTHrP (10⁻⁷M) (Sigma Aldrich; St. Louis, Mo.) was added on day 5 for 24 hours, and RNA was harvested on day 6. For recombinant Col 11A1 experiments, DNA encoding fragments reflecting alternatively spliced products were amplified, ligated into expression vectors and expressed as previously described (64). Recombinant proteins were purified, refolded, and characterized (20, 64). Recombinant Col 11A1 amino terminal domain (NTD) fragments were added to C2C12 cells in culture at a concentration of 30 μg/mL.

Transfection of Cells with Small Interfering RNA

C2C12 cells were grown to 70-80% confluency. Media was then removed and cells were rinsed with phosphate buffered saline (PBS) twice and 2 mL of serum-free DMEM was added into each well of a 6-well plate. Col 11A1 or SMAD 4 siRNA and scrambled control siRNA (10 μM) (Life Technologies, Carlsbad, Calif.) were diluted in 300 μL serum free OPTIMEM (Life Technologies, Carlsbad, Calif.) and mixed with nine microliters of RNAiMAX Lipofectamine (Life Technologies, Carlsbad, Calif.) diluted in 300 μL serum-free OPTIMEM. The mixture was incubated at room temperature for 15 min and then 250 μL of mixture was added into each well of a 6-well plate that already contained 1.75 mL of serum-free DMEM. Cells were incubated with siRNA-containing medium for 24 hours at 37° C. in a humidified chamber containing 5% CO₂. The final concentration of the siRNA was 15 nM. A fluorescent siRNA oligonucleotide (siGLO, Dharmacon, Lafayette, Colo.) was used to confirm efficient delivery of siRNA to 71%±5.1 (SD) of the cells. After treatment, RNA was extracted as described below to confirm Col 11a1 knockdown and to determine the expression levels of osteoblast markers. Scramble control siRNA experiments were carried out in an identical manner to account for any non-specific effects. Cells were treated with BMP-2 (300 ng/mL) for 24 h and 72 h after the 24 h transfection to analyze the effect of diminished Col 11A1 levels on BMP-2 signaling activity. For BMP-2 treatment, after the 24 h Col 11A1 siRNA transfection, media was replaced with DMEM containing 5% FBS and BMP-2 (300 ng/mL) and incubated for 24 h and 72 h. RNA was extracted as described below.

### Semi-Quantitative Polymerase Chain Reaction

Total RNA was extracted from cells using TriZol (Gibco-BRL; Grand Island, N.Y.) and 2 μg of RNA was used to synthesize cDNA using High-Capacity cDNA Reverse Transcript Kit with RNase inhibitor (Life Technologies, Carlsbad, Calif.). Twenty-five microliter PCR reactions were prepared using 12.5 μL GoTaq Colorless Master Mix (Promega, Madison, Wis.), 1 μL of each forward and reverse primer (10 μM), 3 μL, undiluted cDNA and 7.5 μL, nuclease-free water. Samples were amplified for 32 cycles, with denaturation at 95° C. (3 min), annealing at 57° C. (1 min) and extension at 72° C. (30 sec). Col 11A1 forward primer was designed to anneal within exon 5 and the reverse to anneal within exon 9, flanking the variable region: 5′-CAG GAG CCG CAC ATA GAT GAG-3′ (forward), 5′-TTT CTC TCC ATA TGC GCC AT-3′ (reverse), generating a defined set of PCR products reflecting the alternative splicing patterns of the specific cell type. To account for any difference in the amount of RNA, glyceraldehyde 3-phosphate dehydrogenase (GAPDH) was chosen as an endogenous control. The amplification products were separated by electrophoresis through 4% Nusieve 3:1 Agarose (Lonza, Basel, Switzerland) gels according to manufacturer's instructions and visualized under UV light after staining with ethidium bromide.

### Primer Design and Quantitative Real-Time PCR

Primers were designed using Primer Blast (NCBI) (Table 5). Real-time PCR was performed using SYBR Green PCR Master Mix (Applied Biosystems, Life Technologies; Carlsbad, Calif.). Each 20 μL PCR reaction consisted of 10 μL SYBR Green PCR Master Mix, 500 nM of each forward and reverse primer, and 1 μL of undiluted template cDNA plus nuclease-free water. Targets were amplified using an Eppendorf real time PCR Mastercycler as following: 50° C. (2 min), 95° C. (10 min) followed by 40 cycles of 95° C. (15 sec) and 60° C. (1 min) followed by one cycle of 72° C. (1 min). Expression levels were quantified relative to housekeeping gene levels of expression and presented as 2^{-ACT} values to reflect the ratio of expression level of the gene of interest to that of the housekeeping gene. PCR products were separated by electrophoresis through 4% Nusieve 3:1 Agarose (Lonza, Basel, Switzerland) to verify that the primer pair produced a single product of the expected size. A ‘no-template’ control was also included in the assay substituting water for template as well as a control omitting reverse transcriptase to control for the possibility of genomic DNA contamination.

### Western Blot Analysis

To confirm SMAD phosphorylation upon BMP-2 treatment, C2C12 cells were plated at 2×10⁴ cells/cm² and grown overnight. The next day, medium was changed to DMEM containing 5% FBS and BMP-2, and incubated for 30 minutes. Cells were then rinsed twice with cold PBS and lysed using 250 μL of cold M-PER lysis buffer (Pierce, Rockford, Ill.) containing protease and phosphatase inhibitors (Halt Protease Inhibitor™, Pierce, Rockford, Ill.). A 25 μl aliquot was used to determine protein concentration using a BCA assay (Bio Rad; Hercules, Calif.). Twenty micrograms of protein samples were combined with NuPAGE LDS sample buffer and β-mercaptoethanol (Life Technologies; Carlsbad, Calif.) and incubated at 70° C. for 10 minutes. Proteins were then separated by 4-12% SDS-PAGE gradient gel and transferred to nitrocellulose membranes using iBlot® 7-minute iblotting system (Life Technologies; Carlsbad, Calif.). Membranes were blocked for 1 hr at room temperature using SuperBlock solution (Life Technologies, Carlsbad, Calif.), washed 3×5 min with 10 mM Tris, pH 8 containing 150 mM NaCl and 0.05% Tween-20 (TBST) while gently rocking and probed with antibodies as described (Table 6) overnight at 4° C. After 3×5 min washes with TBST, horseradish peroxidase-conjugated donkey anti-rabbit IgG (1:5000 dilution in blocking buffer, Cell Signaling, Danvers, Mass.) was added for 1 hr at room temperature. Blots were then washed 3×5 min with TBST and Incubated in SuperSignal West Femto Maximum Sensitivity Substrate™ (Pierce, Rockford, Ill.) for 1 min and exposed for 5 min on a Kodak Imager 4000R imaging system. Constant substrate concentration was achieved by acquiring images while the nitrocellulose membrane was completely submerged in ECL solution. ECL images were taken in the native format using the system's standard software KODAK and exported to 16-bit TIFF format. Analysis of ECL images was performed using the public domain Image) program (developed at the National Institutes of Health and available at http://rsb.info.nih.gov.libproxy.boisestate.edu/ij/), using the “Gel Analysis” functions. Background correction was done using “background subtractor” tool. Integrated Density Value (IDV) of each band was used to quantify the signals.

### Immunofluorescence

C2C12 cells were plated on sterile glass coverslips in the presence and absence of BMP-2 (300 ng/mL). Media was removed and cells were rinsed twice with PBS. Cells were then fixed with 1:1 ice-cold acetone:methanol for 15 min and rinsed twice with PBS. Coverslips were then washed 3×5 min with PBS while gently rocking and then blocked with 2% BSA for 1 hour at room temperature. Antibodies specific for SMAD 1 and phospho-SMAD 1/5/8 were used at the indicated dilutions (Table 6) in 0.5% BSA in PBS (PBB) and coverslips were incubated at 4° C. overnight while gently rocking. Cells were subsequently washed 5×5 min in PBB. A Rhodamine (TRITC)-conjugated AffiniPure Donkey Anti-rabbit IgG (2.5 μg/mL) (Jackson ImmunoResearch Laboratories) in PBB was added to the cells and incubated at room temperature in the dark for 1 hour. Coverslips were then washed 5×5 min with PBB and mounted on slides using ProLong® with DAPI (Life Technologies; Carlsbad, Calif.). Images were acquired using an LSM Meta 510 scanning confocal microscope (Zeiss, Germany) and ZEN 2009 imaging software (Carl Zeiss, Inc., Thornwood, N.Y.). A pinhole of 1.5 Airy units and objective of 63× oil (NA 1.4) was used. Excitation at 405 nm for DAPI and 543 nm for Rhodamine red-X allowed visualization of nuclei, SMAD 1 and phospho-SMAD 1/5/8. Confocal stacks of 12 optical sections with an optical section separation (z-interval) of 1.18 μm were acquired at 512×512 pixels. Equivalent settings were used for all images.

### Luciferase Reporter Assays

To determine the effect of Col 11A1 on BMP activity, cells were plated at 6.5×10⁴ cells/well of a 24-well plate and incubated overnight. The following day, cells were washed twice with PBS and transfected with LT1 liposomes (Mirus, Madison, Wis.) containing 10 ng/well of CMV β-galactosidase control plasmid and 300 ng/well of BMP reporter element luciferase plasmid (a kind gift from Dr. Allan Albig, Boise, Id.) for 24 hours. In designated experiments, cells were also transfected with 14 ng/well of Silencer Select Col 11A1 siRNA (Life Technologies; Carlsbad, Calif.). The next day, cells were washed with PBS and medium was changed to DMEM supplemented with 5% FBS and treated with Col 11A1 recombinant protein fragments and BMP-2 (300 ng/mL) for 24 h. The following day, C2C12-BRE-luc cells were lysed in Reporter Lysis Buffer (Promega, Madison, Wis.) and frozen overnight. Lysates were then collected and enzyme activity was measured using the Luciferase Assay System and β-Galactosidase Enzyme Assay System (Promega, Madison, Wis.). Enzyme activities were measured in a microplate luminometer. The ratio of luciferase to β-galactosidase activity was calculated, and fold-induction was determined relative to control. Each data point represents the mean of results from three independent transfections.

### Results

### Col 11A1-Deficient Mice Exhibit Disruption in the Mineralization of Primary Trabeculae and Bone Collar.

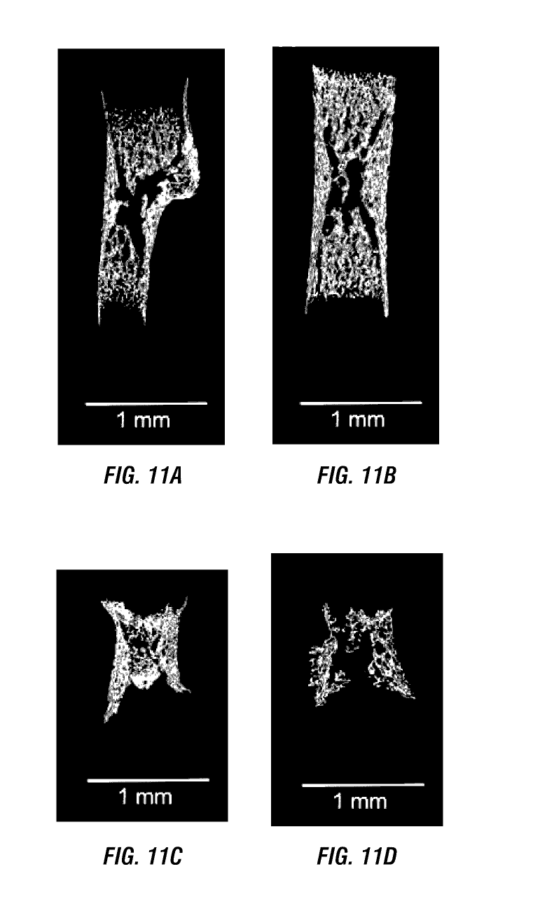

The Col 11A1-deficient long bones displayed an overgrowth of mineralized tissue on the outer surface of the bone collar as well as within the mineralized zone adjacent to the hypertrophic zone (FIG. 11). Trabecular thickness, trabecular separation and percent bone volume were increased in the bones of the Col 11A1-deficient mice compared to wildtype. (Hafez et al. in press).

### BMP-2 Induces the Expression of Col 11A1 During Osteoblast Differentiation in a SMAD-Dependent Manner.

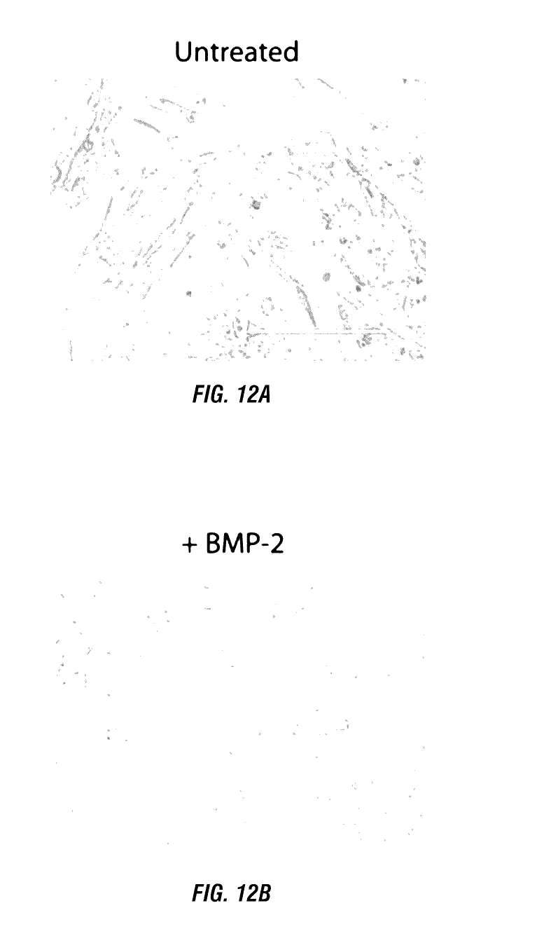

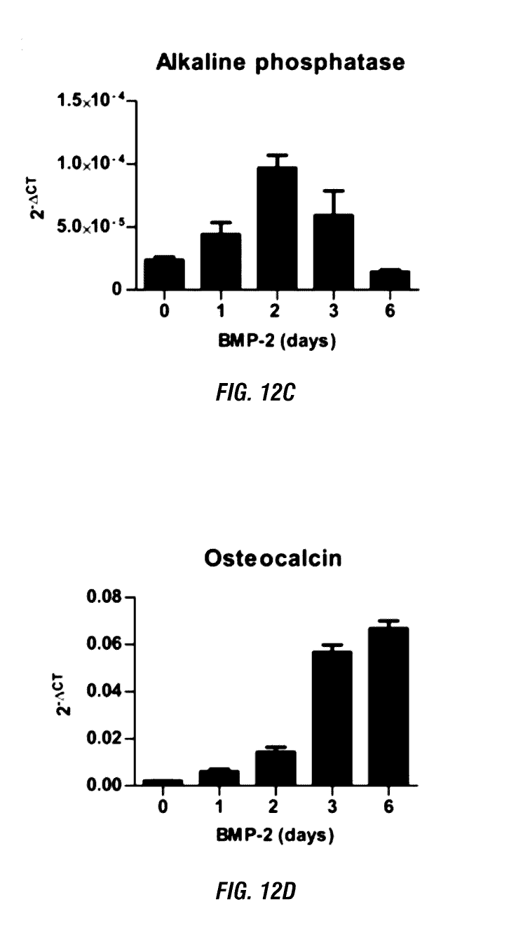

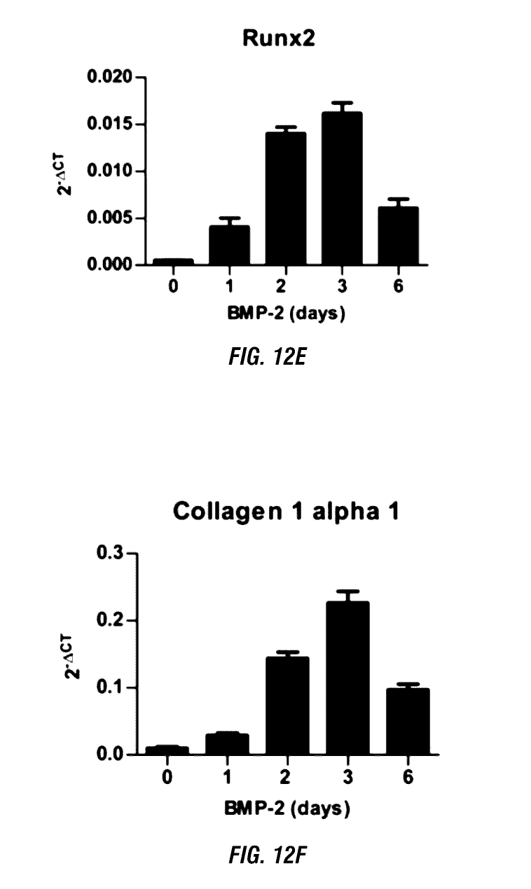

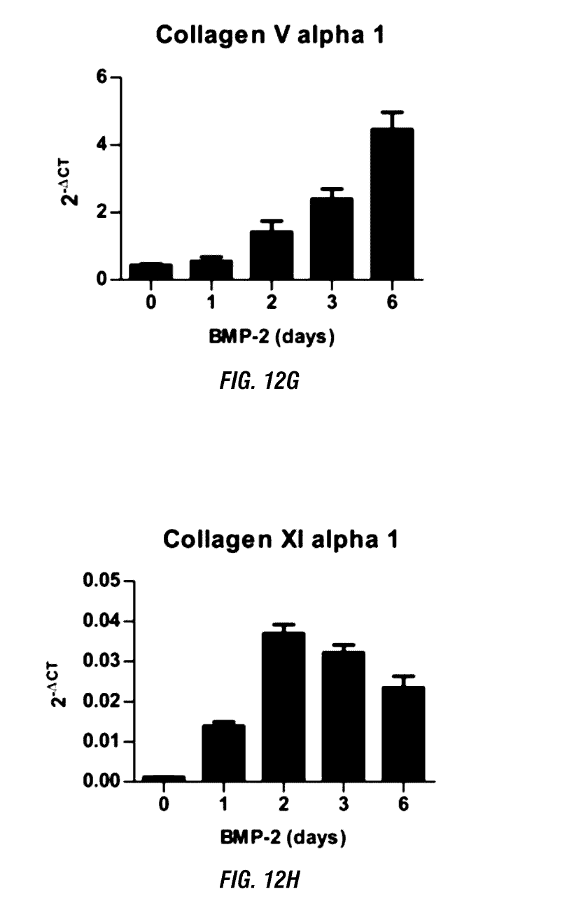

BMP-2 regulates periosteal bone formation during development and we have previously shown that Col 11A1 plays a role in alkaline phosphatase (ALP) expression (33, 59). To evaluate a potential role for BMP-2 in the regulation of Col 11A1 expression during osteoblast differentiation, we used the pluripotent mesenchymal C2C12 cell line. When treated with BMP-2, C2C12 cells changed their morphology from spindle to cuboidal-shaped cells (FIG. 12A and FIG. 12B). To confirm that BMP-2 induced osteoblast differentiation under our culture conditions, we assessed the expression of well-established osteoblast markers ALP, osteocalcin (OCN), runt-related transcription factor 2 (Runx2), and collagen I alpha 1 (Col 1a1). Our results showed an increase in early osteoblast differentiation marker ALP up to day 2 (FIG. 12C). Similarly, Runx2 and Col 1a1 expression increased up to day 3 followed by a decrease on day 6, while late osteoblast marker OCN levels persistently increased reaching maximum expression on day 6 (FIG. 12D, FIG. 12E, and FIG. 12F). Further, BMP-2 induced the expression of Col 5a1 in a time-dependent manner during osteoblast differentiation (FIG. 12G).

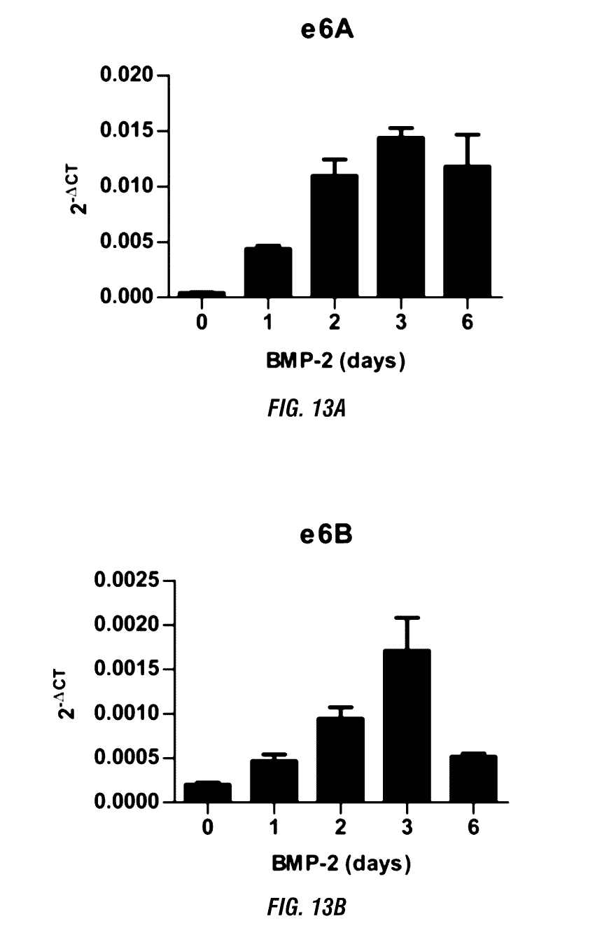

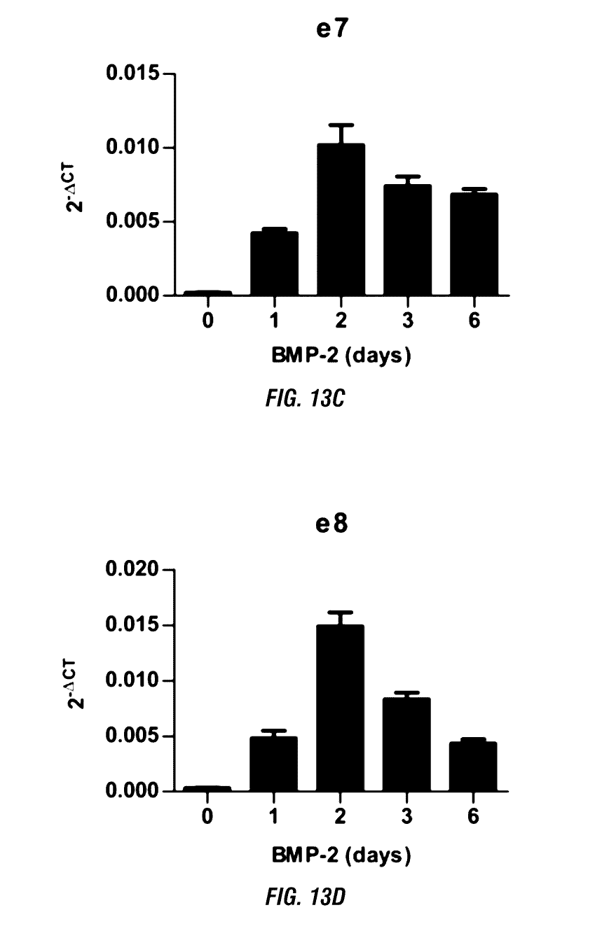

To determine if BMP-2 induced Col 11A1 expression in C2C12 cells during osteoblast differentiation, we treated cells with BMP-2 over a time course of 6 days. We analyzed expression levels of Col 11A1 as well as alternative exon expression on days 1, 2, 3, and 6 by quantitative real-time PCR. Our results demonstrated an induction of Col 11A1 expression by BMP-2 (FIG. 12H). An early increase was observed for exon 6A and this increase persisted up to day 6 (FIG. 13A). The expression of exons 7 and 8 increased up to 2-3 days and then decreased or leveled off (FIG. 13C and FIG. 13D). Exon 6B expression was very low compared to other exons and only showed a slight increase on day 3 (FIG. 13B).

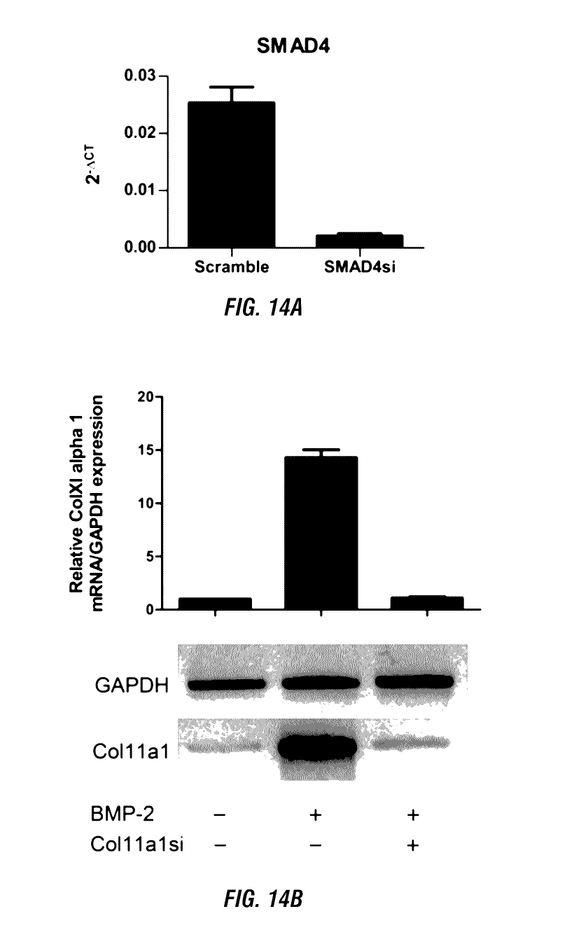

To elucidate whether induction of Col 11A1 gene expression by BMP-2 was dependent upon the canonical SMAD signaling pathway, we knocked down SMAD 4 in C2C12 cells using SMAD 4 siRNA and Lipofectamine (FIG. 14A). SMAD 4 knockdown eliminated the ability of BMP-2 to induce Col 11A1 gene expression (FIG. 14B) demonstrating that regulation of Col 11A1 expression by BMP-2 was dependent on the canonical SMAD 1/5/8 signaling pathway.

### PTHrP Modulates BMP-2-Induced Changes in Col 11A1 Expression.

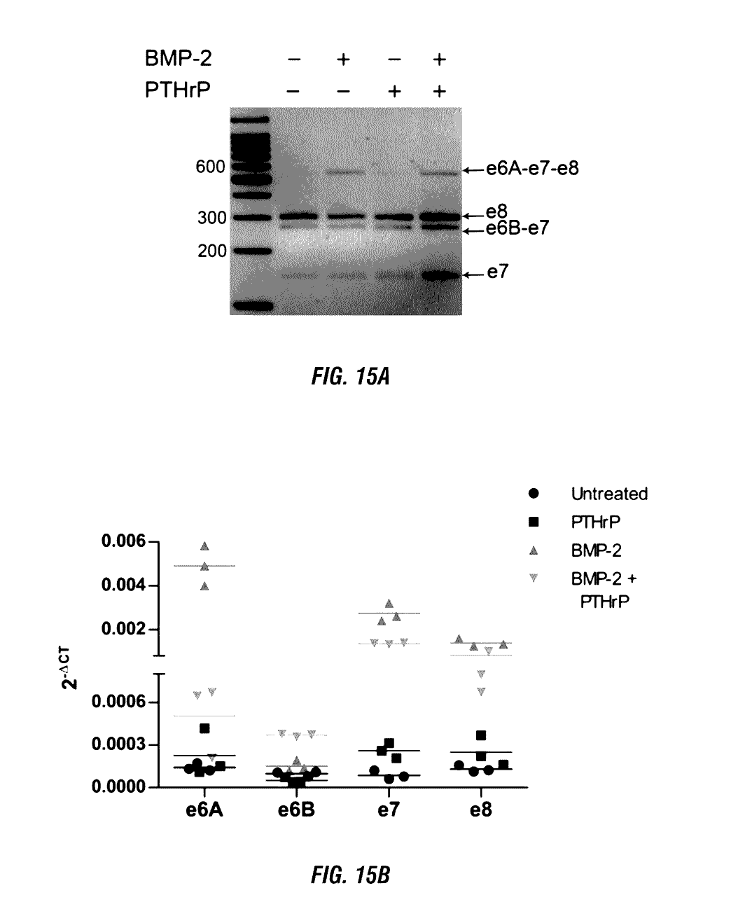

Previous studies demonstrated an inhibitory role for PTHrP (1-36) on BMP-2-induced osteoblast marker expression (40), and thus we sought to investigate the effects of PTHrP on BMP-2-induced Col 11A1 expression. Differentiating osteoblasts expressed four predominant splice forms containing variable region exons in the following combinations: 1) e6A-e7-e8, 2) e8, 3) 6B-e7, and 4) e7 (FIG. 15A). Quantitative real time PCR demonstrated that PTHrP attenuated the BMP-2-induced inclusion of exons 6A, 8, and 7 (FIG. 15B). PTHrP alone did not have significant effects on Col 11A1 splice form expression at 24 h continuous treatment.

### Col 11A1 is Required for BMP-2 Induction of Osteoblast Markers During Early Osteoblastogenesis.

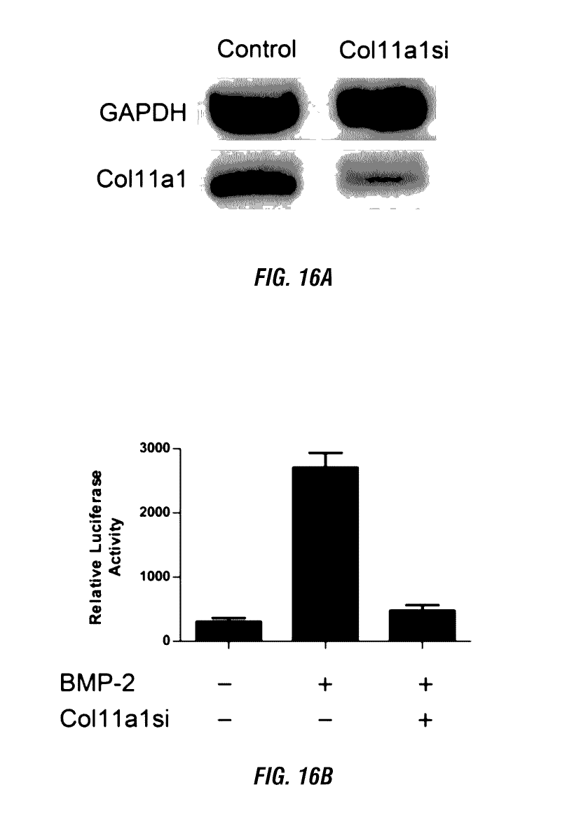

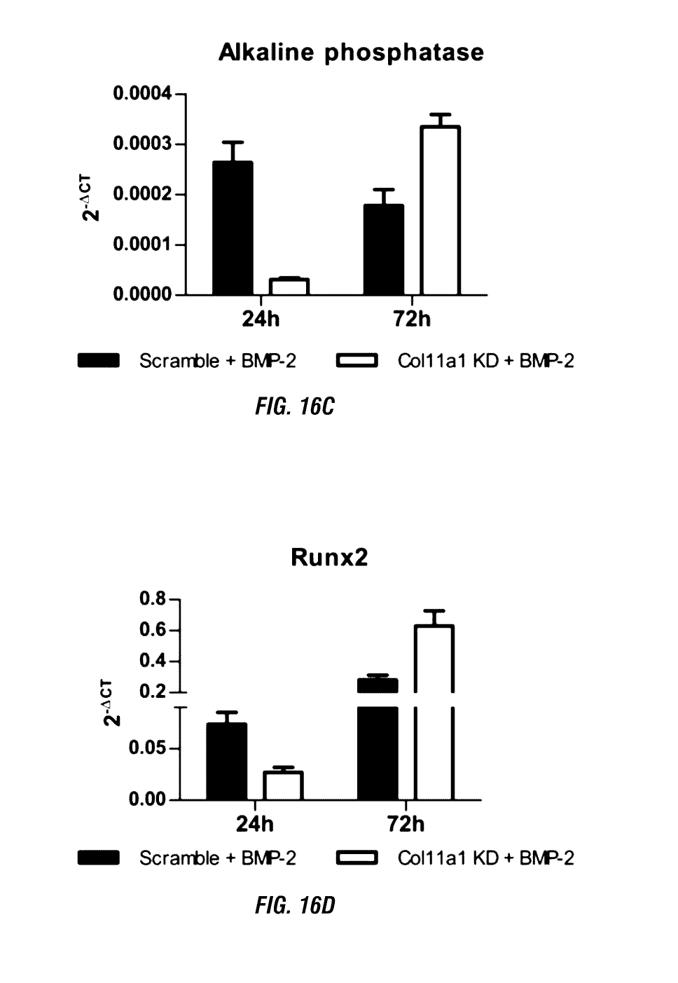

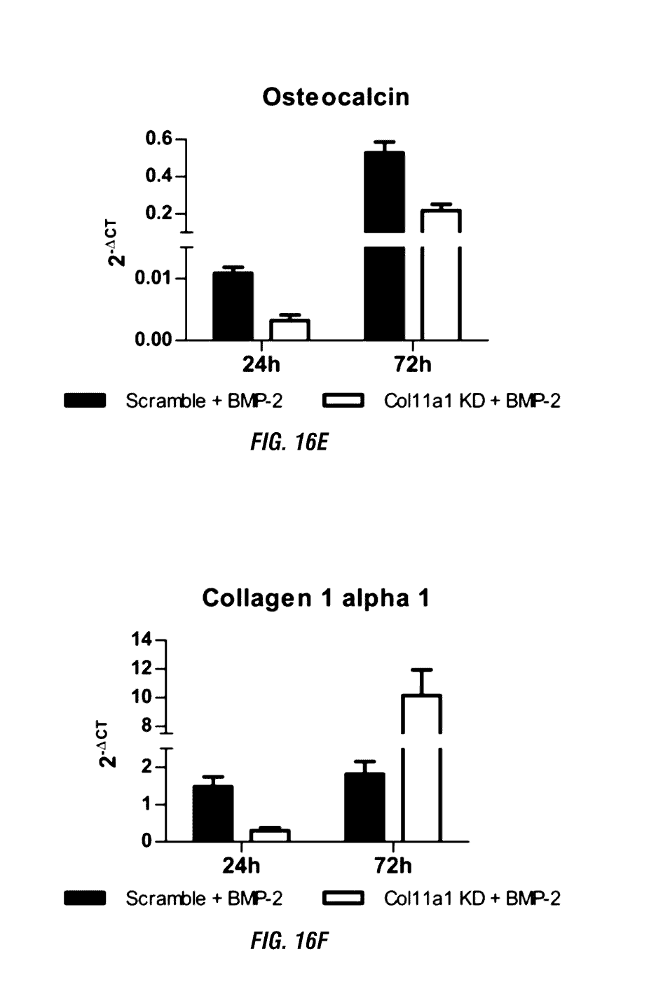

To further elucidate the biological relevance of Col 11A1 during osteoblast differentiation, we assessed the effects of Col 11a1 knockdown on BMP-2-induced expression of osteoblast markers ALP, OCN, Runx2, and Col 1a1. Upon transfecting C2C12 cells with either Col 11A1 siRNA or control scramble siRNA, we confirmed our knockdown by RT-PCR (FIG. 16A). We tested the effects of Col 11A1 on BMP signaling, using a luciferase BMP-response element reporter construct. Treatment of C2C12 cells with BMP-2 induced an increase in luciferase activity, indicating activation of BMP signaling compared to untreated control cells. Activation of BMP-2 signaling was dependent upon Col 11A1, as Col 11A1 siRNA reduced the BMP activity by 82% (p-value 0.0161) compared to controls in C2C12 cells during the first 24 hours (FIG. 16B). We then determined the expression levels of osteoblast markers using quantitative real-time PCR. Our results demonstrated that Col 11A1 knockdown caused a decrease in 24 h BMP-2-stimulated osteoblast marker expression including OCN, Col 1a1, Runx2, and most markedly for ALP (FIG. 16 (C-F)). In contrast, this effect was reversed later at 72 h osteoblast differentiation. After 72 hours of BMP-2 treatment, Col 11A1 siRNA increased the expression of ALP, Runx2, and Col 1a1 genes by 1.8 fold (p-value <0.01), 2.2 fold (p-value <0.01), and 5.6 fold (p-value <0.001), respectively compared to scramble siRNA (FIG. 16C, FIG. 16D, and FIG. 16F). Interestingly, OCN expression level remained suppressed in the presence of Col 11A1 siRNA compared to scramble siRNA at 72 h, similar to the effect seen at 24 hours (FIG. 16E).

### Col 11A1 is Required for BMP-2-Induced SMAD1/5/8 Phosphorylation.

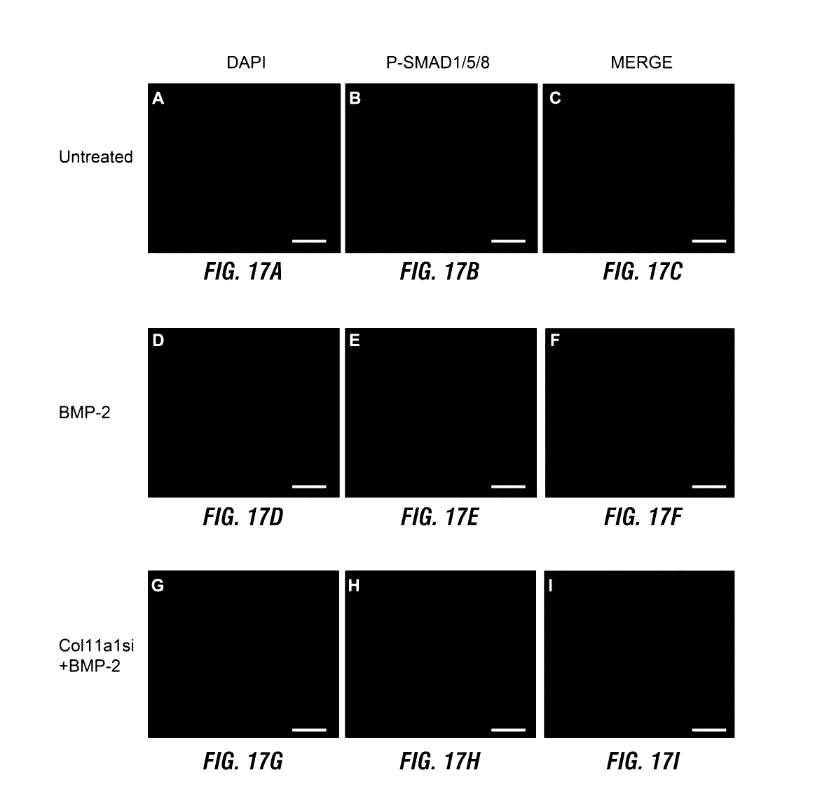

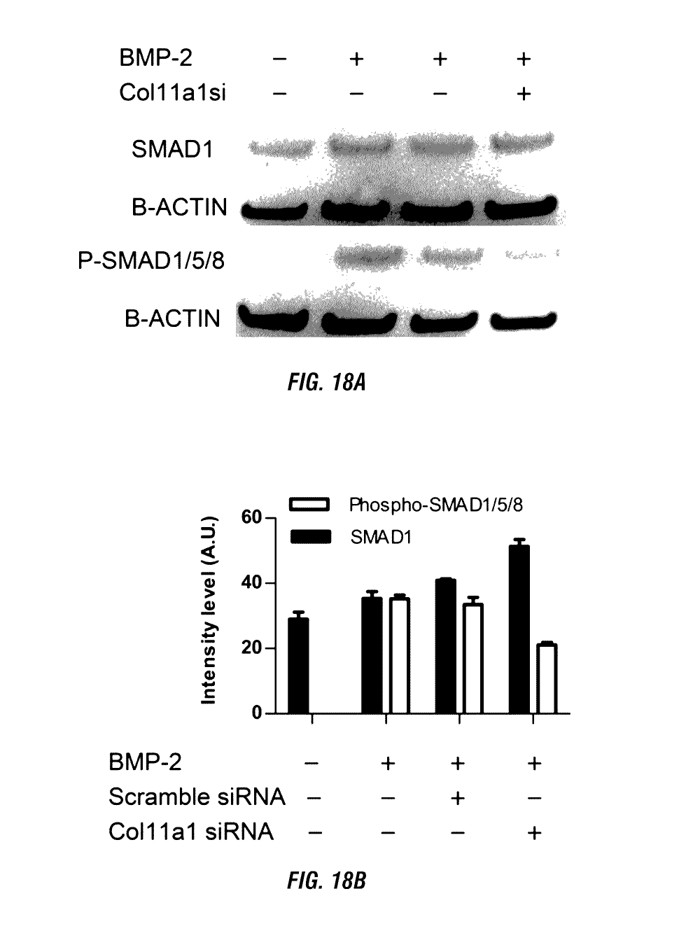

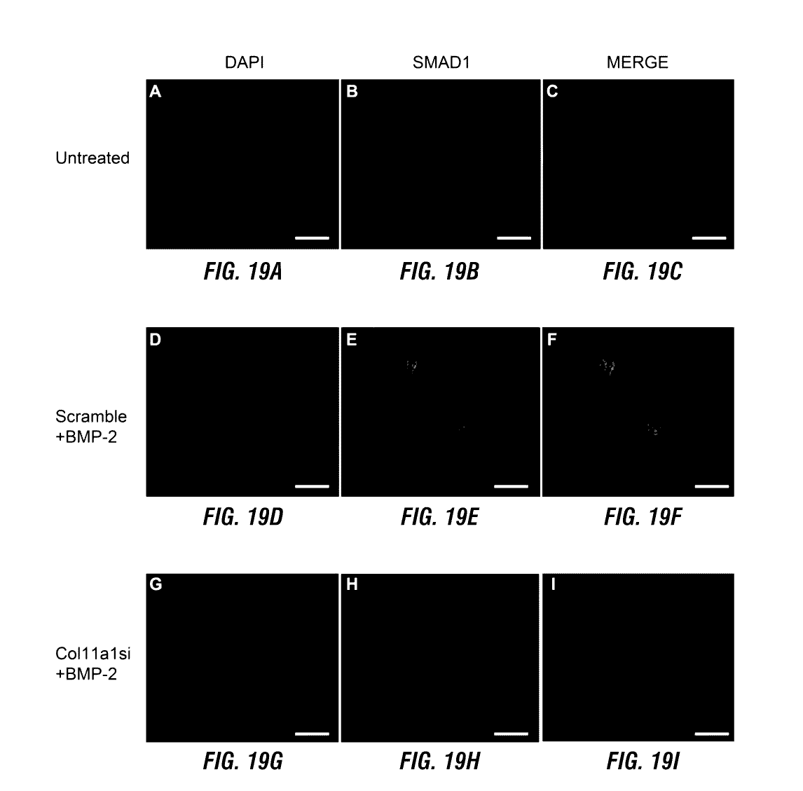

To elucidate the mechanism by which Col 11A1 regulates BMP-2-induced expression of osteoblast markers, we investigated the role of Col 11A1 in SMAD 1/5/8 phosphorylation and nuclear translocation in C2C12 cells by western blot and immunocytochemistry. Treatment with BMP-2 induced SMAD 1/5/8 phosphorylation, which was not detected in control cells (FIG. 17 (A-F)). Further, phospho-SMAD 1/5/8 co-localized with DAPI, indicating its presence in the nucleus (FIG. 17F). Pre-treatment with Col 11A1 siRNA for 24 hours inhibited the translocation to the nucleus of phospho-SMAD 1/5/8 (FIG. 17 (G-I)). Pretreatment with Col 11A1 siRNA for 24 hours resulted in a reduction of phospho-SMAD 1/5/8 by approximately 50% (p-value <0.001) (FIG. 18A and FIG. 18B). Total SMAD 1/5/8 was also observed to change dependent upon the presence of Col 11a1 (FIG. 19 (A-I)). Treatment with BMP-2 resulted in an increase of total SMAD 1 detected within the cells (FIG. 19B compared to FIG. 19E), and much of the signal was found to co-localize with the nuclear stain DAPI (FIG. 19F). The increase in total SMAD 1 and the nuclear localization was prevented in the absence of Col 11A1 expression (FIG. 19 (G-I)). Instead, perinuclear accumulation was observed for the total SMAD 1 present in the cell in the absence of Col 11A1 (FIG. 19I). A 45% (p-value <0.01) increase in the ratio of total SMAD 1 protein to β-actin protein was observed in the Col 11A1-deficient cells compared to cells treated with the control scramble siRNA (FIG. 18B).

Recombinant Col 11A1 [p7-8] NTD Fragment Enhances BMP-2-Induction of Specific Osteoblast Markers at 24 Hours.

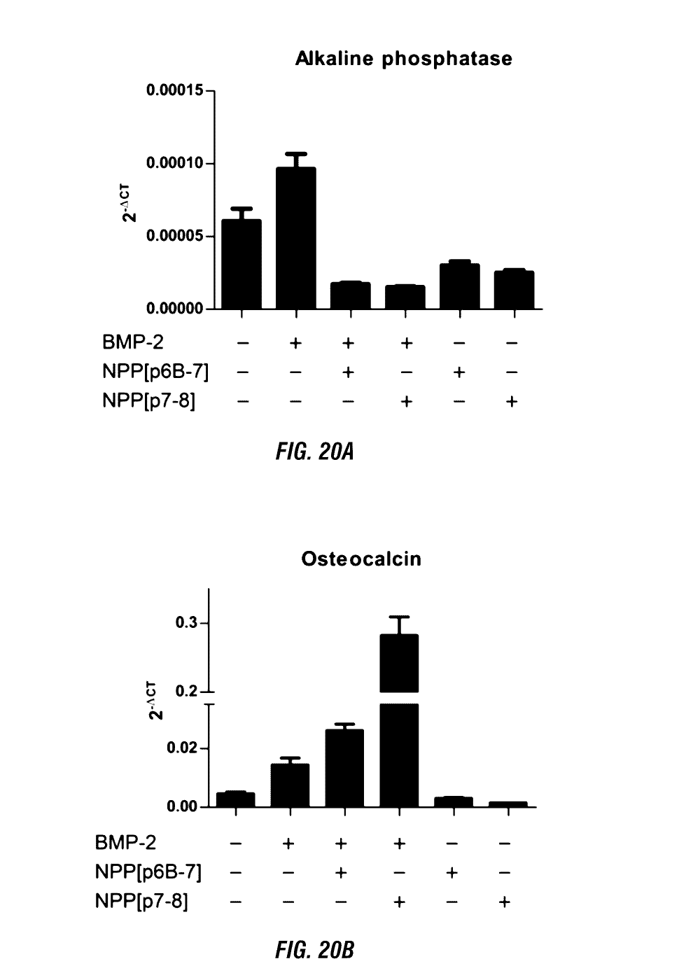

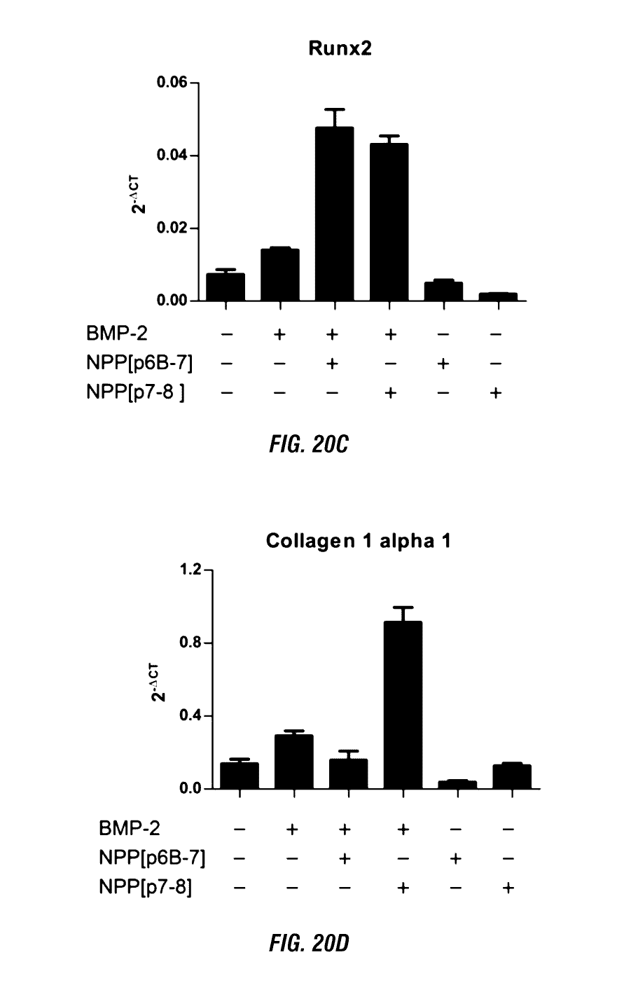

An essential role for Col 11A1 during the first 24 hours of BMP-2 treatment was further demonstrated by treating cells with recombinant Col 11A1 [p7-8] NTD fragment, representing a predominant splice form synthesize by C2C12 cells that includes exons 7 and 8 but omits exons 6A and 6B, as shown above. Treatment with this recombinant fragment of Col 11A1 resulted in a decrease in ALP expression by 6.3 fold (p-value 0.0143) (FIG. 20A), similar to results observed for recombinant Col 11A1 [p6B-7] NTD fragment verified here as control and shown previously (65). In contrast however, treatment with recombinant Col 11A1 [p7-8] NTD fragment resulted in increased levels of expression for OCN (19.5 fold, p-value 0.0103), Runx2 (3.0 fold, p-value 0.0099), and Col 1a1 (3.1 fold, p-value 0.0284) (FIG. 20 (B-D)). An isoform-specific effect for recombinant Col 11A1 [p7-8] NTD fragment on BMP-2 induction was observed for the expression of Col 1a1 and for OCN, compared to the recombinant Col 11A1[p6B-7] NTD fragment. This difference between Col 11A1 isoforms may reflect the spatiotemporal regulation of alternative splicing during endochondral ossification and bone collar formation, as Col 11A1[p6B-7] is restricted to the region immediately adjacent to the new forming bone collar, forming a boundary between the cartilage growth plate and the surrounding bone collar. As expected, recombinant Col 11A1[p6B-7] NTD fragment decreased the BMP-2-induced ALP expression by 5.5 fold (p-value 0.0132) and Col 1a1 expression by 1.8 fold (p-value 0.032) consistent with an inhibitory role for early osteoblast marker expression (FIG. 20A and FIG. 20D). Conversely, the addition of recombinant Col 11A1[p6B-7] NTD fragment promoted the expression of OCN by 1.8 fold (p-value 0.017) and Runx2 by 3.4 fold (p-value 0.022) in BMP-2-induced cells consistent with a positive regulatory role for late osteoblast differentiation (FIG. 20B and FIG. 20C).

### Col 11A1[p6B-7] NTD Fragment Inhibits BMP-2 Activity in C2C12 Cells.

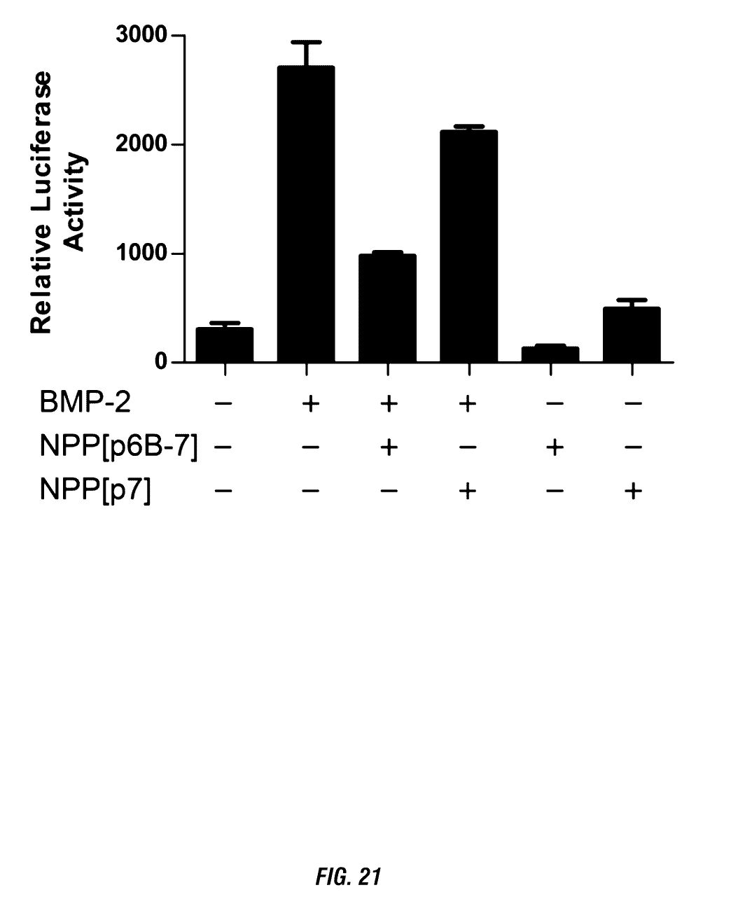

Col 11A1[p6B-7] is specifically expressed by chondrocytes located immediately adjacent to the developing bone collar during endochondral ossification (24). To investigate the potential role of Col 11A1[p6B-7] on BMP-2 activity in osteoblasts, we used a luciferase BMP-response element reporter construct. As expected, treatment with BMP-2 increased luciferase activity compared to control untreated cells (FIG. 21). Treatment with recombinant Col 11A1[p6B-7] NTD fragment inhibited the BMP-2 induction of luciferase activity by 64% (p-value 0.0136).

Conversely, this effect was not observed when cells were treated with a recombinant Col 11A1 [p7] NTD fragment, demonstrating an exon 6B-specific effect. These two splice forms differ only by the absence or presence of 51 amino acids encoded by exon 6B, indicating a specific function in the regulation of growth plate maturation, specifically BMP-2 induced activity in neighboring osteoblasts.

### Discussion

These findings indicate that Col 11A1 splice forms play specific roles in the regulation of C2C12 osteoblast differentiation. The effects of individual splice forms may be in opposition with respect to the other splice forms, which gives Col 11A1 a unique ability to balance the progression of bone collar formation in coordination with endochondral ossification based on the regulation of alternative splicing. We propose Col 11A1 as a coupling factor between endochondral ossification and bone collar formation.

Col 11A1 is an extracellular matrix protein that is essential for proper skeletal development. Marshall and Stickler syndrome patients carry heterozygous mutations in Col 11A1 and suffer from short stature and skeletal abnormalities. Previous groups have shown that Col 11A1-deficient mice exhibit increased skeletal mineralization compared to their wildtype littermates (12, 14). Based on recent microarray studies showing increased Col 11A1 expression during differentiation, we hypothesized that Col 11A1 would affect periosteal bone architecture and further alter osteoblast differentiation in a BMP-dependent manner.

Our findings support a role for Col 11A1 in bone formation consistent with alterations to the bone collar during skeletal development. Further, we have presented information that suggests intersection between BMP-2 signaling and Col 11A1-regulation of bone formation. Our results indicate that BMP-2 regulates Col 11A1 transcription and alternative splicing of pre-mRNA characterized by the expression of exons 6A, 7, and 8 in osteoblasts. BMP-2-stimulated expression of exon 6B on day 3 coincided with a significant decrease in the expression levels of osteoblast markers ALP, Runx2, and Col 1a1. We also showed that exogenous treatment of cells with recombinant Col 11A1 [p6B-7] NTD fragment significantly reduced BMP-2 activity. One possible explanation for reduced BMP-2 activity upon exposure to recombinant Col 11A1 [p6B-7] NTD fragment is its direct binding between BMP-2 and Col 11A1 or an indirect interaction via heparan sulfate on the surface of osteoblasts, which are responsible for trapping and internalizing BMP-2 into the cell (18, 66, 67). Our laboratory has previously shown that Col 11A1 binds specifically to heparan sulfate and heparan sulfate proteoglycans via the p6B region and Npp domain (18).

Treatment of C2C12 cells with recombinant Col 11A1[p6B-7] NTD fragment resulted in a significant decrease in ALP and Col 1a1 expression but not OCN and Runx2. Instead, recombinant Col 11A1 [p6B-7] NTD fragment induced increased expression of OCN and Runx2. This finding is intriguing because although OCN is up-regulated by BMP-2 and is essential for osteoblast differentiation, OCN-deficient mice exhibit increased bone formation suggesting a limiting role for OCN in bone formation (68). Furthermore, previous studies have shown that osteocalcin is under direct regulation of Runx2 in osteoblasts (69). Thus, it is possible that Col 11A1[p6B] acts upstream of Runx2 and induces the expression of Runx2 and OCN to limit bone formation. Alternatively, since Runx2 is a shared target of BMP and TGF-β signaling pathways (70), Col 11A1 [p6B]-mediated inhibition of BMP signaling at the receptor level may signal the cells to increase TGF-β-induced expression of Runx2 and subsequently OCN. Since BMP-2 increased the inclusion and expression of exon 8, we postulate a positive regulatory role for regions of the protein encoded by exons 6A, 7, and 8 during osteoblast differentiation. Indeed, when we treated BMP-2-stimulated cells with recombinant Col 11A1 [p7-8] NTD fragment, mRNA levels of OCN, Runx2, and Col 1a1 increased significantly. Surprisingly though, ALP expression was decreased by the addition of recombinant Col 11A1 [p7-8] NTD fragment.

Upon knocking down Col 11A1, we initially observed a decrease in the expression of osteoblast markers, suggesting that Col 11A1 was required for BMP-2 mediated induction of osteoblast marker expression. However, by 72 h, we noted the opposite trend and detected higher levels of osteoblast marker expression (except for OCN) in Col 11A1-deficient cells as compared to control. This difference in the trend may be due to 1) compensatory mechanism used by cells to increase their expression of osteoblast markers and overcome the Col 11A1 knockdown and/or 2) early induction of additional genes that have yet to be identified, perhaps other BMPs or receptors.

Continuous treatment with PTHrP has been shown to inhibit osteoblast differentiation (57). Previous teams have shown a feedback mechanism between BMP-2 and PTHrP during osteoblast differentiation, in which PTHrP attenuates BMP-2-induced increases in osteoblast markers Runx2 and Osterix (40). Our findings indicate a regulatory role for PTHrP in BMP-2-induced alternative splicing of Col 11A1, further supporting our postulation of exons 6A, 7, and 8 as newly identified markers of osteoblast differentiation and positive regulators of BMP-2 mediated osteoblast differentiation. We also reported an increase in Col 11A1 exon 6B expression when PTHrP was added to BMP-2 treated cells, although this effect did not reach statistical significance.

Canonical BMP signaling requires phosphorylation of SMAD 1/5/8 and thus we wanted to test whether Col 11A1 knockdown affects this process. Our results clearly show that Col 11A1-deficient cells exhibit lower levels of phospho-SMAD 1/5/8 as compared to control cells.

Furthermore, phospho-SMAD 1/5/8 proteins in Col 11A1-deficient cells are primarily localized to the cytoplasm and not localized to the nucleus.

In conclusion, our study clearly demonstrates a splice form-specific novel function for Col 11A1 as a regulator of BMP-2-induced osteoblast signaling and osteoblast differentiation. These findings are consistent with our earlier studies suggesting that Col 11A1[p6B-7] acts to inhibit ALP expression during osteoblast differentiation (59), and further extends our previous studies in a significant way to yield additional mechanistic details. Based on our observations, we propose a model, in which BMP-2 regulates the alternative splicing of Col 11A1, and in response, specific splice forms of Col 11A1 act to either inhibit or enhance BMP-2 signaling and downstream expression of osteoblast markers correlated with the spatial and temporal expression patterns within the developing long bone.

Future studies will address the role of Col 11A1 in cell proliferation, direct binding interactions between different collagen splice forms and BMP-2 and the effects of Col 11A1 on integrin-mediated BMP-2 induction of osteoblast differentiation (71). These additional studies will offer insight into how Col 11A1 couples endochondral ossification to bone collar formation during bone development.

### REFERENCES

1. Kronenberg H M (2006) PTHrP and skeletal development. Ann N Y Acad Sci 1068:1-13.
2. Ricard-Blum S (2011) The collagen family. Cold Spring Harb Perspect Biol 3(1):a004978.
3. Annunen S, et al. (1999) Splicing mutations of 54-bp exons in the COL11A1 gene cause Marshall syndrome, but other mutations cause overlapping Marshall/Stickler phenotypes. Am J Hum Genet 65(4):974-83.
4. Tompson S W, et al. (2012) Dominant and recessive forms of fibrochondrogenesis resulting from mutations at a second locus, COL11A2. Am J Med Genet A 158A(2):309-14.
5. Vijzelaar R, et al. (2013) Deletions within COL11A1 in Type 2 stickler syndrome detected by multiplex ligation-dependent probe amplification (MLPA). BMC Med Genet 14:48.
6. Akawi N a. Ali B R, Al-Gazali L (2013) A response to Dr. Alzahrani's letter to the editor regarding the mechanism underlying fibrochondrogenesis. Gene 528(2):367-8.
7. Acke F R, et al. (2014) Novel pathogenic COL11A1/COL11A2 variants in Stickier syndrome detected by targeted NGS and exome sequencing. MOl Genet Metab 113(3):230-235.
8. Griffith A J, Gebarski S S, Shepard N T, Kileny P R (2000) Audiovestibular phenotype associated with a COL11A1 mutation in Marshall syndrome. Arch Otolaryngol Head Neck Surg 126:891-894.
9. Wu J-J, Weis M A, Kim L S, Carter B G, Eyre D R (2009) Differences in chain usage and cross-linking specificities of cartilage type V/XI collagen isoforms with age and tissue. J Biol Chem 284(9):5539-45.
10. Niyibizi C, Eyre D R (1994) Structural characteristics of cross-linking sites in type V collagen of bone. Chain specificities and heterotypic links to type I collagen. Eur J Biochem 224(3):943-50.
11. Niyibizi C, Eyre D R (1989) Identification of the cartilage alpha 1(XI) chain in type V collagen from bovine bone. FEBS Lett 242(2):314-8.
12. Seegmiller R, Fraser F C, Sheldon H (1971) A new chondrodystrophic mutant in mice. Electron microscopy of normal and abnormal chondrogenesis. J Cell Biol 48(3):580-93.
13. Fernandes R J, Weis M, Scott M a, Seegmiller R E, Eyre D R (2007) Collagen XI chain misassembly in cartilage of the chondrodysplasia (cho) mouse. Matrix Biol 26(8):597-603.
14. Li Y, et al. (1995) A Fibrillar Collagen Gene, Col11a 1, Is Essential for Skeletal Morphogenesis. Cell 80:423-430.
15. McGuirt W T, et al. (1999) Mutations in COL11A2 cause non-syndromic hearing loss (DFNA13). Nat Genet 23:413-419.
16. Li S W, et al. (2001) Targeted disruption of Col11a2 produces a mild cartilage phenotype in transgenic mice: Comparison with the human disorder otospondylomegaepiphyseal dysplasia (OSMED). Dev Dyn 222:141-152.
17. Ryan M C, Sandell L J (1990) Differential expression of a cysteine-rich domain in the amino-terminal propeptide of type II (cartilage) procollagen by alternative splicing of mRNA. J Biol Chem 265:10334-10339.
18. Warner L R, Brown R J, Yingst S M C, Oxford J T (2006) Isoform-specific heparan sulfate binding within the amino-terminal noncollagenous domain of collagen alpha1(XI). J Biol Chem 281(51):39507-16.
19. Keene D R, Oxford J T, Morris N P (1995) Ultrastructural localization of collagen types II, IX, and XI in the growth plate of human rib and fetal bovine epiphyseal cartilage: type XI collagen is restricted to thin fibrils. J Histochem Cytochem 43(10):967-979.
20. Gregory K E (2000) Structural Organization of Distinct Domains within the Non-collagenous N-terminal Region of Collagen Type XI. J Biol Chem 275(15):11498-11506.
21. Tsumaki N, Kimura T (1995) Differential expression of an acidic domain in the amino-terminal propeptide of mouse pro-??2(XI) collagen by complex alternative splicing. J Biol Chem 270:2372-2378.
22. Davies G B, Oxford J T, Hausafus L C, Smoody B F, Morris N P (1998) Temporal and spatial expression of alternative splice-forms of the alpha1(XI) collagen gene in fetal rat cartilage. Dev Dyn 213(1):12-26.
23. Moradi-Améli M, De Chassey B, Farjanel J, Van Der Rest M (1998) Different splice variants of cartilage α1(XI) collagen chain undergo uniform amino-terminal processing. Matrix Biol 17:393-396.
24. Morris N P, Oxford J T, Davies G B, Smoody B F, Keene D R (2000) Developmentally regulated alternative splicing of the alpha1(XI) collagen chain: spatial and temporal segregation of isoforms in the cartilage of fetal rat long bones. J Histochem Cytochem 48(6):725-41.
25. Brown R J, Mallory C, McDougal O M, Oxford J T (2011) Proteomic analysis of Col11a1-associated protein complexes. Proteomics 11(24):4660-76.
26. Oxford J T, Doege K J, Morris N P (1995) Alternative Exon Splicing within the Amino-terminal Nontriple-helical Domain of the Rat Pro-alpha1(XI) collagen chain generates multiple forms of the mRNA transcript which exhibit tissue-dependent variation. J Biol. Chem 270(21):9478-9485.
27. Valcourt U, Gouttenoire J, Aubert-Foucher E, Herbage D, Mallein-Gerin F (2003) Alternative splicing of type procollagen pre-mRNA in chondrocytes is oppositely regulated by BMP-2 and TGF-β1. FEBS Lett 545(2-3):115-119.
28. McAlinden A, Shim K, Wirthlin L, Ravindran S, Hering T M (2012) Quantification of type II procollagen splice forms using alternative transcript-qPCR (AT-qPCR). Matrix Biol 31(7-8):412-20.
29. Urist M R, Mikulski a, Lietze a (1979) Solubilized and insolubilized bone morphogenetic protein. Proc Natl. Acad Sci USA 76(4):1828-1832.
30. Urist M R, DeLange R J, Finerman G A (1983) Bone cell differentiation and growth factors. Science (80-) 220:680-686.
31. Katagiri T, et al. (2014) Bone morphogenetic protein-induced heterotopic bone formation: What have we learned from the history of a half century? Jpn Dent Sci Rev 51(2):42-50.
32. Caron M M J, et al. (2013) Hypertrophic differentiation during chondrogenic differentiation of progenitor cells is stimulated by BMP-2 but suppressed by BMP-7. Osteoarthr Cartil 21:604-613.
33. Wang Q, Huang C, Xue M, Zhang X (2011) Expression of endogenous BMP-2 periosteal progenitor cells is essential for bone healing. Bone 48:524-532.
34. Samee M, et al, (2008) Bone morphogenetic protein-2 (BMP-2) and vascular endothelial growth factor (VEGF) transfection to human periosteal cells enhances osteoblast differentiation and bone formation. J Pharmacol Sci 108:18-31.
35. Reilly G C, Golden E B, Grasso-Knight G, Leboy P S (2005) Differential effects of ERK and p38 signaling in BMP-2 stimulated hypertrophy of cultured chick sternal chondrocytes. Cell Commun Signal 3:3.
36. Nakahara H, Goldberg V M, Caplan A I (1991) Culture-expanded human periosteal-derived cells exhibit osteochondral potential in vivo. J Orthop Res 9:465-476.
37. Yaffe D, Saxel O (1977) A myogenic cell line with altered serum requirements for differentiation, Differentiation 7:159-166.
38. Katagiri T, et al. (1994) Bone Morphogenetic Protein-2 Converts the Differentiation on Pathway of C2C12 Myoblasts into the Osteoblast Lineage. J Cell Biol 127(6):1-5.
39. Li G, et al. (2005) Differential Effect of BMP4 on NIH/3T3 and C2C12 Cells: Implications for Endochondral Bone Formation. J Bone Miner Res 20(9):1611-1623.
40. Susperregui ARG, et al. (2008) BMP-2 regulation of PTHrP and osteoclastogenic factors during osteoblast differentiation of C2C12 cells. J Cell Physiol 216(1):144-52.
41. Lagunas A, et al. (2013) Continuous bone morphogenetic protein-2 gradients for concentration effect studies on C2C12 osteogenic fate. Nanomedicine Nanotechnology. Biol Med 9:694-701.
42. Yoon B S, et al. (2006) BMPs regulate multiple aspects of growth-plate chondrogenesis through opposing actions on FGF pathways. Development 133(23):4667-78. McCauley L K, et al. (1996) PTH/PTHrP receptor is temporally regulated during osteoblast differentiation and is associated with collagen synthesis. J Cell Biochem 61(4):638-47.
43. Hirai T, Chagin A S, Kobayashi T, Mackem S, Kronenberg H M (2011) Parathyroid hormone/parathyroid hormone-related protein receptor signaling is required for maintenance of the growth plate in postnatal life. Proc Natl Acad Sci USA 108(1):191-6.
44. Van Donkelaar C C, Huiskes R (2007) The PTHrP-Ihh feedback loop in the embryonic growth plate allows PTHrP to control hypertrophy and Ihh to regulate proliferation. Biomech Model Mechanbiol 6:55-62.
45. Martin T J (2005) Osteoblast-derived PTHrP is a physiological regulator of bone formation. J Clin Invest 115:2322-2324.
46. Datta N S, Chen C, Berry J E, McCauley L K (2005) PTHrP signaling targets cyclin D1 and induces osteoblastic cell growth arrest. J Bone Miner Res 20(6):1051-64.
47. Kamel S A, Yee J A (2013) Continuous and intermittent exposure of neonatal rat calvarial cells to PTHrP (1-36) inhibits bone nodule mineralization in vitro by downregulating bone sialoprotein expression via the cAMP signaling pathway. F1000Res 2:77.
48. Dean T, Vilardaga J-P, Potts J T, Gardella T J (2008) Altered selectivity of parathyroid hormone (PTH) and PTH-related protein (PTHrP) for distinct conformations of the PTH/PTHrP receptor. Mol Endocrinol 22(1):156-66.
49. Horwitz M J, et al. (2005) Continuous PTH and PTHrP infusion causes suppression of bone formation and discordant effects on 1,25(OH)2 vitamin D. J Bone Miner Res 20:1792-1803.
50. Horwitz M J, et al. (2011) A 7-day continuous infusion of PTH or PTHrP suppresses bone formation and uncouples bone turnover. J Bone Miner Res 26:2287-2297.
51. Carpio L, Gladu J, Goltzman D, Rabbani S A (2001) Induction of osteoblast differentiation indexes by PTHrP in MG-63 cells involves multiple signaling pathways. Am J Physiol Endocrinol Metab 281(3): E489-E499.
52. Zhang K, et al. (2014) Effects of parathyroid hormone-related protein on osteogenic and adipogenic differentiation of human mesenchymal stem cells. Eur Rev Med Pharmacol Sci 18(11):1610-7.
53. Casado-Diaz A, Santiago-Mora R, Quesada J M (2010) The N- and C-terminal domains of parathyroid hormone-related protein affect differently the osteogenic and adipogenic potential of human mesenchymal stem cells. Exp Mol Med 42(2):87-98.
54. Zerega B, Cermelli S, Bianco P, Cancedda R, Cancedda E D (1999) Parathyroid hormone [PTH(1-34)] and parathyroid hormone-related protein [PTHrP(1-34)] promote reversion of hypertrophic chondrocytes to a prehypertrophic proliferating phenotype and prevent terminal differentiation of osteoblast-like cells. J Bone Miner Res 14(8):1281-9.
55. Pellicelli M, et al. (2012) PTHrP(1-34)-mediated repression of the PHEX gene in osteoblastic cells involves the transcriptional repressor E4BP4. J Cell Physiol 227(6):2378-87.
56. van der Horst G. Farih-Sips H, Löwik C W G M, Karperien M (2005) Multiple mechanisms are involved in inhibition of osteoblast differentiation by PTHrP and PTH in KS483 Cells. J Bone Miner Res 20(12):2233-44.
57. Li T-F, et al. (2004) Parathyroid hormone-related peptide (PTHrP) inhibits Runx2 expression through the PKA signaling pathway. Exp Cell Res 299(1):128-36.
58. Kahler R A, et al. (2008) Collagen 11a1 is indirectly activated by lymphocyte enhancer-binding factor 1 (Lef1) and negatively regulates osteoblast maturation. Matrix Biol 27:330-338.
59. Twine N a, Chen L, Pang C N, Wilkins M R, Kassem M (2014) Identification of differentiation-stage specific markers that define the ex vivo osteoblastic phenotype. Bone 67:23-32.
60. Evans K N, et al. (2014) Osteogenic gene expression correlates with development of heterotopic ossification in war wounds. Clin Orthop Relat Res 472(2):396-404.
61. Neuss S. et al. (2011) Transcriptome analysis of MSC and MSC-derived osteoblasts on Resomer® LT706 and PCL: impact of biomaterial substrate on osteogenic differentiation. PLoS One 6(9):e23195.
62. Lui J C, et al. (2012) Synthesizing genome-wide association studies and expression microarray reveals novel genes that act in the human growth plate to modulate height. Hum Mol Genet 21:5193-5201.
63. Warner L R, Blasick C M, Brown R J, Oxford J T (2007) Expression, purification, and refolding of recombinant collagen alpha1(XI) amino terminal domain splice variants. Protein Expr Purif 52(2):403-9.
64. Kahler R a, et al. (2008) Collagen 11a1 is indirectly activated by lymphocyte enhancer-binding factor 1 (Lef1) and negatively regulates osteoblast maturation. Matrix Biol 27(4):330-8.
65. Jiao X, et al. (2007) Heparan Sulfate Proteoglycans (HSPGs) modulate BMP2 osteogenic bioactivity in C2C12 cells. J Biol Chem 282:1080-1086.
66. Bramono D S, et al. (2012) Bone marrow-derived heparan sulfate potentiates the osteogenic activity of bone morphogenetic protein-2 (BMP-2). Bone 50(4):954-964.
67. Ducy P, et al. (1996) Increased hone formation in osteocalcin-deficient mice. Nature 382(6590):448-452.
68. Jana W G, et al. (2012) BMP2 protein regulates osteocalcin expression via Runx2-mediated Atf6 gene transcription. J Biol Chem 287(2):905-915.
69. Lee K S, et al. (2000) Runx2 is a common target of transforming growth factor beta1 and bone morphogenetic protein 2, and cooperation between Runx2 and Smad5 induces osteoblast-specific gene expression in the pluripotent mesenchymal precursor cell line C2C12. Mol Cell Biol 20(23):8783-8792. Jikko a, Harris S E, Chen D, Mendrick D L, Damsky C H (1999) Collagen integrin receptors regulate early osteoblast differentiation induced by BMP-2. J Bone Miner Res 14(7):1075-1083.

TABLE 5
Real-time PCR primers were designed to amplify osteoblast
markers and different Col11a1 exons within the variable
region of the NTD using NCBI Primer Blast. Primers were
purchased from Integrated DNA Technologies and
resuspended in nuclease-free water. All primers were used
at annealing temperature of 60° C. For maximum
efficiency, PCR amplicon sizes were kept below 200 by and
products were analyzed. using agarose gel electrophoresis
to validate amplification product size and homogeneity.

| GENE | SENSE PRIMER | ANTISENSE PRIMER |
|---|---|---|
| ALP | GTGCCCTGACTGAGGCTGTC | GGATCATCGTGTCCTGCTCAC |
| | (SEQ ID NO: 7) | (SEQ ID NO: 8) |
| COL1A1 | GCATGGCCAAGAAGACATCC | CCTCGGGTTTCCACGTCTC (SEQ |
| | (SEQ ID NO: 9) | ID NO: 10) |
| COL11A1 e6A | AGGCTGAGAGTGTAACAGAGA | TCTGTTTGTGCTACTGTTTCTTC |
| | (SEQ ID NO: 11) | A (SEQ ID NO: 12) |
| COL11A1 e6B | GTTCACATCCCCCAAATCTGA | CCCCTAGTTTGGCTTTGGCT (SEQ |
| | (SEQ ID NO: 13) | ID NO: 14) |
| COL11A1 e7 | GGAACAATGGAACCTTACCAGA | ATTCGATCCTGATACCCGCC (SEQ |
| | C (SEQ ID NO: 15) | ID NO: 16) |
| COL11A1 e8 | AGGAGTAGACGGCAGGGATT | GGAGGTCGTAGTCCTTTCTTCA |
| | (SEQ ID NO: 17) | (SEQ ID NO: 18) |
| OSTEOCALCIN | CCGGGAGCAGTGTGAGCTTA | TAGATGCGTTTGTAGGCGGTC |
| | (SEQ ID NO: 19) | (SEQ ID NO: 20) |
| PPIA | CGCGTCTCCTTCGAGCTGTTTG | TGTAAAGTCACCACCCTGGCACA |
| | (SEQ ID NO: 21) | T (SEQ ID NO: 22) |
| RUNX2 | GTGCGGTGCAAACTTTCTCC | AATGACTCGGTTGGTCTCGG |
| | (SEQ ID NO: 231 | (SEQ ID NO: 241 |

TABLE 6
Antibodies were purchased from Cell Signaling and used at the
indicated dilutions in blocking buffer overnight at 4° C.

| | | | | DILU- | | SPECI- |
|---|---|---|---|---|---|---|
| NAME | COMPANY | CAT# | ISOTYPE | TION | MW | FICITY |
| Beta- | Cell | 4967 | Rabbit | 1:5000 | 45 | Poly- |
| Actin | Signaling | | | | kDa | clonal |
| Smad1 | Cell | 6944 | Rabbit | 1:1000 | 60 | Mono- |
| (D59D7) | Signaling | | IgG | | kDa | clonal |
| Phospho- | Cell | 9516 | Rabbit | 1:1000 | 60 | Mono- |
| Smad1/5 | Signaling | | IgG | | kDa | clonal |
| (41D10) | | | | | | |

### Example 3

| Sequence of recombinant protein: SEQ ID NO: 1 | | |
|---|---|---|
| 1 | ASPVDILKALDFHNSPVGISKTTGFCTSRKNSKDPDIAYRVTEEAQISAPTKQLFPGGIF | 60 |
| 61 | PQDFSILFTIKPKKGTQAFLLSLYNEHGIQQLGVEVGRSPVFLFEDHTGKPTPENYPLFS | 120 |
| 121 | TVNIADGKWHRVAISVEKKTVTMIVDCKKKITKFLDRSERSIVDTNGIMVFGTRILETDV | 180 |
| 181 | FQGDIQQFLITGDPKAAYDYCDHYSPDCDLTSPKAAQAQBPHIDEKKKSNYTKKKRTLAT | 240 |
| 241 | NSKKKSKMSTTPKSEKFASKKKKRNQATAKAKLGVQANIVDDFQDYNYGTMETYQTESPR | 300 |
| 301 | RVSGSNEINGHGAYGEKGQKGEPAVVE | 327 |

| Collagen alpha-1(XI) chain isoform B preproprotein [Homo sapiens]. | |
|---|---|
| 1818 aa protein. This variant (B) utilizes alternate exon 6, | |
| designated exon 6B, and encodes the longest isoform (B). | |
| Accession: NP_542196.2 GI: 98985810 (SEQ ID NO: 2) | |
| sig_peptide | 1 . . . 36 |
| | /calculated_mol_wt = 4416 |
| proprotein | 37 . . . 1818 |
| | /product = “collagen alpha-1(XI) chain isoform B proprotein” |
| | /calculated_mol_wt = 178025 |
| Region | 73 . . . 225 |
| | /region_name = “LamG” |
| | /note = “Laminin G domain; Laminin G-like domains are |
| | usually Ca++ mediated receptors that can have binding |
| | sites for steroids, beta1 integrins, heparin, sulfatides, |
| | fibulin-1, and alpha-dystroglycans. Proteins that contain |
| | LamG domains serve a variety of . . . ; cd00110” |
| | /db_xref = “CDD: 238058” |
| mat_peptide | 524 . . . 1574 |
| | /product = “collagen alpha-1(XI) chain isoform B” |
| | /calculated_mol_wt = 97473 |

| 1 | mepwssrwkt krwlwdftvt tlaltflfqa revrgaapvd vlkaldfhns pegiskttgf |
|---|---|
| 61 | ctnrknskgs dtayrvskqa qlsaptkqlf pggtfpedfs ilftvkpkkg iqsfllsiyn |
| 121 | ehgiqqigve vgrspvflfe dhtgkpaped yplfrtvnia dgkwhrvais vekktvtmiv |
| 181 | dckkkttkpl drseraivdt ngitvfgtri ldeevfegdi qqflitgdpk aaydycehys |
| 241 | pdcdssapka aqaqepqide kkksnfkkkm rtvatkskek skkftppkse kfsskkkksy |
| 301 | qasakaklgv kanivddfqe ynygtmesyq teaprhvsgt nepnpveeif teeyltgedy |
| 361 | dsqrknsedt lyenkeidgr dsdllvdgdl geydfyeyke yedkptsppn eefgpgvpae |
| 421 | tditetsing hgaygekgqk gepavvepgm lvegppgpag pagimgppgl qgptgppgdp |
| 481 | gdrgppgrpg lpgadglpgp pgtmlmlpfr yggdgskgpt isaqeaqaqa ilqqarialr |
| 541 | gppgpmgltg rpgpvggpgs sgakgesgdp gpqgprgvqg ppgptgkpgk rgrpgadggr |
| 601 | gmpgepgakg drgfdglpgl pgdkghrger gpqgppgppg ddgmrgedge igprglpgea |
| 661 | gprgllgprg tpgapgqpgm agvdgppgpk gnmgpqgepg ppgqqgnpgp qglpgpqgpi |
| 721 | gppgekgpqg kpglaglpga dgppghpgke gqsgekgalg ppgpqgpigy pgprgvkgad |
| 781 | gvrglkgskg ekgedgfpgf kgdmglkgdr gevgqigprg edgpegpkgr agptgdpgps |
| 841 | gqagekgklg vpglpgypgr qgpkgstgfp gfpgangekg argvagkpgp rgqrgptgpr |
| 901 | gsrgargptg kpgpkgtsgg dgppgppger gpqgpqgpvg fpgpkgppgp pgkdglpghp |
| 961 | gqrgetgfqg ktgppgpggv vgpqgptget gpigerghpg ppgppgeqgl pgaagkegak |
| 1021 | gdpgpqgisg kdgpaglrgf pgerglpgaq gapglkggeg pqgppgpvgs pgergsagta |
| 1081 | gpiglpgrpg pqgppgpage kgapgekgpq gpagrdgvqg pvglpgpagp agspgedgdk |
| 1141 | geigepgqkg skgdkgengp pgppglqgpv gapgiaggdg epgprgqqgm fgqkgdegar |
| 1201 | gfpgppgpig lqglpgppge kgengdvgpm gppgppgprg pqgpngadgp qgppgsvgsv |
| 1261 | ggvgekgepg eagnpgppge agvggpkger gekgeagppg aagppgakgp pgddgpkgnp |
| 1321 | gpvgfpgdpg ppgepgpagq dgvggdkged gdpgqpgppg psgeagppgp pgkrgppgaa |
| 1381 | gaegrqgekg akgeagaegp pgktgpvgpq gpagkpgpeg lrgipgpvge qglpgaagqd |
| 1441 | gppgpmgppg lpglkgdpgs kgekghpgli gligppgeqg ekgdrglpgt qgspgakgdg |
| 1501 | gipgpagplg ppgppglpgp qgpkgnkgst gpagqkgdsg lpgppgspgp pgeviqplpi |
| 1561 | lsskktrrht egmqadaddn ildysdgmee ifgslnslkq diehmkfpmg tqtnpartck |
| 1621 | dlqlshpdfp dgeywidpnq gcsgdsfkvy cnftsggetc iypdkksegv risswpkekp |
| 1681 | gswfsefkrg kllsyldveg nsinmvqmtf lklltasarq nftyhchqsa awydvssgsy |
| 1741 | dkalrflgsn deemsydnnp fiktlydgca srkgyektvi eintpkidqv pivdvmindf |
| 1801 | gdqnqkfgfe vgpvcflg |
| Collagen, type XI, alpha 1, isoform CRA_c [Homo sapiens]. | |
| 1818 aa protein. GenBank: EAW72911.1 (SEQ ID NO: 3) | |
| 1 | mepwssrwkt krwlwdftvt tlaltflfqa revrgaapvd vlkaldfhns pegiskttgf |
| 61 | ctnrknskgs dtayrvskqa qlsaptkqlf pggtfpedfs ilftvkpkkg iqsfllsiyn |
| 121 | ehgiqqigve vgrspvflfe dhtgkpaped yplfrtvnia dgkwhrvais vekktvtmiv |
| 181 | dckkkttkpl drseraivdt ngitvfgtri ldeevfegdi qqflitgdpk aaydycehys |
| 241 | pdcdssapka aqaqepqide kkksnfkkkm rtvatnskek skkftppkse kfsskkkksy |
| 301 | qasakaklgv kanivddfqe ynygtmesyq teaprhvsgt nepnpveeif teeyltgedy |
| 361 | dsqrknsedt lyenkeidgr dsdllvdgdl geydfyeyke yedkptsppn eefgpgvpae |
| 421 | tditetsing hgaygekgqk gepavvepgm lvegppgpag pagimgppgl qgptgppgdp |
| 481 | gdrgppgrpg lpgadglpgp pgtmlmlpfr yggdgskgpt isaqeaqaqa ilqqarialr |
| 541 | gppgpmgltg rpgpvggpgs sgakgesgdp gpqgprgvqg ppgptgkpgk rgrpgadggr |
| 601 | gmpgepgakg drgfdglpgl pgdkghrger gpqgppgppg ddgmrgedge igprglpgea |
| 661 | gprgllgprg tpgapgqpgm agvdgppgpk gnmgpqgepg ppgqqgnpgp qglpgpqgpi |
| 721 | gppgekgpqg kpglaglpga dgppghpgke gqsgekgalg ppgpqgpigy pgprgvkgad |
| 781 | gvrglkgskg ekgedgfpgf kgdmglkgdr gevgqigprg edgpegpkgr agptgdpgps |
| 841 | gqagekgklg vpglpgypgr qgpkgstgfp gfpgangekg argvagkpgp rgqrgptgpr |
| 901 | gsrgargptg kpgpkgtsgg dgppgppger gpqgpqgpvg fpgpkgppgp pgkdglpghp |
| 961 | gqrgetgfqg ktgppgpggv vgpqgptget gpigerghpg ppgppgeqgl pgaagkegak |
| 1021 | gdpgpqgisg kdgpaglrgf pgerglpgaq gapglkggeg pqgppgpvgs pgergsagta |
| 1081 | gpiglpgrpg pqgppgpage kgapgekgpq gpagrdgvqg pvglpgpagp agspgedgdk |
| 1141 | geigepgqkg skgdkgengp pgppglqgpv gapgiaggdg epgprgqqgm fgqkgdegar |
| 1201 | gfpgppgpig lqglpgppge kgengdvgpm gppgppgprg pqgpngadgp qgppgsvgsv |
| 1261 | ggvgekgepg eagnpgppge agvggpkger gekgeagppg aagppgakgp pgddgpkgnp |
| 1321 | gpvgfpgdpg ppgepgpagq dgvggdkged gdpgqpgppg psgeagppgp pgkrgppgaa |
| 1381 | gaegrqgekg akgeagaegp pgktgpvgpq gpagkpgpeg lrgipgpvge qglpgaagqd |
| 1441 | gppgpmgppg lpglkgdpgs kgekghpgli gligppgeqg ekgdrglpgt qgspgakgdg |
| 1501 | gipgpagplg ppgppglpgp qgpkgnkgst gpagqkgdsg lpgppgppgp pgeviqplpi |
| 1561 | lsskktrrht egmqadaddn ildysdgmee ifgslnslkq diehmkfpmg tqtnpartck |
| 1621 | dlqlshpdfp dgeywidpnq gcsgdsfkvy cnftsggetc iypdkksegv risswpkekp |
| 1681 | gswfsefkrg kllsyldveg nsinmvqmtf lklltasarq nftyhchqsa awydvssgsy |
| 1741 | dkalrflgsn deemsydnnp fiktlydgca srkgyektvi eintpkidqv pivdvmindf |
| 1801 | gdqnqkfgfe vgpvcflg |

### Comparison of Sequences

Sequence ID: gb|EAW72911.1|Length: 1818Number of Matches: 2
See 1 more title(s)
Related Information
Gene-associated gene details
Identical Proteins-Proteins identical to the subject
Range 1: 36 to 344GenPeptGraphicsNext MatchPrevious Match

| Score | Expect | Method | Identities | Positives | Gaps [symbol] |
|---|---|---|---|---|---|
| 516 bits(1328) | 2e−167 | Compositional matrix adjust. | 257/309 (83%) | 287/309 (92%) | 0/30 [symbol] |

| | | | | |
|---|---|---|---|---|
| Query | 1 | ASPVDILKALDFHNSPVGISKTTGFCTSRKNSKDPDIAYRVTEEAQISAPTKQLFPGGIF | 60 | |
| | | A+PVD+LKALDFHNSP GISKTTGFCT+RKNSK D AYRV+++AQ+SAPTKQLFPGG F | | |
| Sbjct | 36 | AAPVDVLKALDFHNSPEGISKTTGFCTNRKNSKGSDTAYRVSKQAQLSAPTKQLFPGGTF | 95 | |
| Query | 61 | PQDFSILFTIKPKKGTQAFLLSLYNEHGIQQLGVEVGRSPVFLFEDHTGKPTPENYPLFS | 120 | |
| | | P+DFSILFT+KPKKG Q+FLLS+YNEHGIQQ+GVEVGRSPVFLFEDHTGKP PE+YPLF | | |
| Sbjct | 96 | PEDFSILFTVKPKKGIQSFLLSIYNEHGIQQIGVEVGRSPVFLFEDHTGKPAPEDYPLFR | 155 | |
| Query | 121 | TVNIADGKWHRVAISVEKKTVTMIVDCKKKITKPLDRSERSIVDTNGIMVFGTRILETDV | 180 | |
| | | TVNIADGKWHRVAISVEKKTVTMIVDCKKK TKPLDRSER+IVDTNGI VFGTRIL+ +V | | |
| Sbjct | 156 | TVNIADGKWHRVAISVEKKTVTMIVDCKKKTTKPLDRSERAIVDTNGITVFGTRILDEEV | 215 | |
| Query | 181 | FQGDIOQFLITGDPKAAYDYCDHYSPDCDLTSPKAAQAQEPHIDEKKKSNYTKKKRTLAT | 240 | |
| | | F+GDIQQFLITGDPKAAYDYC+HYSPDCD ++PKAAQAQEP IDEKKKSN+ KK RT+AT | | |
| Sbjct | 216 | FEGDIQQFLITGDPKAAYDYCEHYSPDCDSSAPKAAQAQEPQIDEKKKSNFKKKMRTVAT | 275 | |
| Query | 241 | NSKKKSKMSTTPKSEKFASKKKKRNQATAKAKLGVQANIVDDFQDYNYGTMETYQTESPR | 300 | |
| | | NSK+KSK T PKSEKF+SKKKK QA+AKAKLGV+ANIVDDFQ+YNYGTME+YQTE+PR | | |
| Sbjct | 276 | NSKEKSKKFTPPKSEKFSSKKKKSYQASAKAKLGVKANIVDDFQEYNYGTMESYQTEAPR | 335 | |
| Query | 301 | RVSGSNEIN | 309 | (SEQ ID NO: 1) |
| | | VSG+NE N | | |
| sbjct | 336 | KVSGTNEPN | 344 | (SEQ ID NO: 2) |

Range 2: 428 to 447GenPeptGraphicsNext MatchPrevious MatchFirst Match

| Score | Expect | Method | Identities | Positives | Gaps [symbol] |
|---|---|---|---|---|---|
| 44.7 bits(104) | 6e−04 | Compositional matrix adjust. | 20/20 (100%) | 20/20 (100%) | 0/2 [symbol] |

| | | | | |
|---|---|---|---|---|
| Query | 308 | INGHGAYGEKGQKGEPAVVE | 327 | (SEQ ID NO: 1) |
| | | INGHGAYGEKGQKGEPAVVE | | |
| Sbjct | 428 | INGHGAYGEKGQKGEPAVVE | 447 | |

collagen type XI alpha-a isoform B [Homo sapiens]
Sequence ID: gb|AAF04726.1|Length: 1818Number of Matches: 2
Related Information
Gene-associated gene details
Range 1: 36 to 344GenPeptGraphicsNext MatchPrevious Match

| Score | Expect | Method | Identities | Positives | Gaps [symbol] |
|---|---|---|---|---|---|
| 513 bits(1321) | 2e−166 | Compositional matrix adjust. | 256/309 (83%) | 286/309 (92%) | 0/30 [symbol] |

| | | | | |
|---|---|---|---|---|
| Query | 1 | ASPVDILKALDFHNSPVGISKTTGFCTSRKNSKDPDIAYRVTEEAQISAPTKQLFPGGIF | 60 | |
| | | A+PVD+LKALDFHNSP GISKTTGFCT+RKNSK D AYRV+++AQ+SAPTKQLFPGG F | | |
| Sbjct | 36 | AAPVDVLKALDFHNSPEGISKTTGFCTNRKNSKGSDTAYRVSKQAQLSAPTKQLFPGGTF | 95 | |
| Query | 61 | PQDFSILFTIKPKKGTQAFLLSLYNEHGIQQLGVEVGRSPVFLFEDHTGKPTPENYPLFS | 120 | |
| | | P+DFSILFT+KPKKG Q+FLLS+YNEHGIQQ+GVEVGRSPVFLFEDHTGKP PE+YPLF | | |
| Sbjct | 96 | PEDFSILFTVKPKKGIQSFLLSIYNEHGIQQIGVEVGRSPVFLFEDHTGKPAPEDYPLFR | 155 | |
| Query | 121 | TVNIADGKWHRVAISVEKKTVTMIVDCKKKITKPLDRSERSIVDTNGIMVFGTRILETDV | 180 | |
| | | TVNIADGKWHRVAISVEKKTVTMIVDCKKK TKPLDRSER+IVDTNGI VFGTRIL+ +V | | |
| Sbjct | 156 | TVNIADGKWHRVAISVEKKTVTMIVDCKKKTTKPLDRSERAIVDTNGITVFGTRILDEEV | 215 | |
| Query | 181 | FQGDIQQFLITGDPKAAYDYCDHYSPDCDLTSPKAAQAQEPHIDEKKKSNYTKKKRTLAT | 240 | |
| | | F+GDIQQFLITGDPKAAYDYC+HYSPDCD ++PKAAQAQEP IDEKKKSN+ KK RT+AT | | |
| Sbjct | 216 | FEGDIQQFLITGDPKAAYDYCEHYSPDCDSSAPKAAQAQEPQIDEKKKSNFKKKMRTVAT | 275 | |
| Query | 241 | NSKKKSKMSTTPKSEKFASKKKKRNQATAKAKLGVQANIVDDFQDYNYGTMETYQTESPR | 300 | |
| | | SK+KSK T PKSEKF+SKKKK QA+AKAKLGV+ANIVDDFQ+YNYGTME+YQTE+PR | | |
| Sbjct | 276 | KSKEKSKKFTPPKSEKFSSKKKKSYQASAKAKLGVKANIVDDFQEYNYGTMESYQTEAPR | 335 | |
| Query | 301 | RVSGSNEIN | 309 | (SEQ ID NO: 1) |
| | | VSG+NE N | | |
| Sbjct | 336 | HVSGTNEPN | 344 | (SEQ ID NO: 2) |

Range 2: 428 to 447GenPeptGraphicsNext MatchPrevious MatchFirst Match

| Score | Expect | Method | Identities | Positives | Gaps [symbol] |
|---|---|---|---|---|---|
| 44.7 bits(104) | 6e−04 | Compositional matrix adjust. | 20/20 (100%) | 20/20 (100%) | 0/2 [symbol] |

| | | | | |
|---|---|---|---|---|
| Query | 308 | INGHGAYGEKGQKGEPAVVE | 327 | (SEQ ID NO: 1) |
| | | INGHGAYGEKGQKGEPAVVE | | |
| Sbjct | 428 | INGHGAYGEKGQKGEPAVVE | 447 | |

Sequence of recombinant protein: (SEQ ID NO: 1)

| | | |
|---|---|---|
| 1 | ASPVDILKALDFHNSPVGISKTTGFCTSRKNSKDPDIAYRVTEEAQISAPTKQLFPGGIF | 60 |
| 61 | PQDFSILFTIKPKKGTQAFLLSLYKEHGIQQLGVEVGRSPVFLFEDHTGKPTPENYPLFS | 120 |
| 121 | TVNIADGKWHRVAISVEKKTVTMIVDCKKKITKPLDRSERSIVDTNGIMVFGTRILETDV | 180 |
| 181 | FQGDIQQFLITGDPKAAYDYCDHYSPDCDLTSPKAAQAQEPHIDEKKKSNYTKKKRTLAT | 240 |
| 241 | NSKKKSKMSTTPKSEKFASKKKKRNQATAKAKLGVQANIVDDFQDYNYGTMETYQTESPR | 300 |
| 301 | RVSGSNEINGHGAYGEKGQKGEPAVVE | 327 |

Accession number of naturally occurring COL11A1:
collagen alpha-1(XI) chain isoform B preproprotein [Homo sapiens]
1818 aa protein
Accession: NP_542196.2 GI: 98985810
And
collagen, type XI, alpha 1, isoform CRA_c [Homo sapiens]
GenBank: EAW72911.1
Collagen alpha-1(XI) cham isoform A preproprotein [Homo sapiens]; NP_001845.3
(SEQ ID NO: 2)

| | |
|---|---|
| 1 | mepwssrwkt krwlwdftvt tlaltflfqa revrgaapvd vlkaldfhns pegiskttgf |
| 61 | ctnrknskgs dtayrvskqa qlsaptkqlf pggtfpedfs ilftvkpkkg iqsfllsiyn |
| 121 | ehgiqqigve vgrspvflfe dhtgkpaped yplfrtvnia dgkwhrvais vekktvtmiv |
| 181 | dckkkttkpl drseraivdt ngitvfgtri ldeevfegdi qqflitgdpk aaydycehys |
| 241 | pdcdssapka aqaqepqide yapediieyd yeygeaeyke aesvtegptv teetiaqtea |
| 301 | nivddfqeyn ygtmesyqte aprhvsgtne pnpveeifte eyltgedyds qrknsedtly |
| 361 | enkeidgrds dllvdgdlge ydfyeykeye dkptsppnee fgpgvpaetd itetsinghg |
| 421 | aygekgqkge pavvepgmlv egppgpagpa gimgppglqg ptgppgdpgd rgppgrpglp |
| 481 | gadglpgppg tmlmlpfryg gdgskgptis aqeaqaqail qqarialrgp pgpmgltgrp |
| 541 | gpvggpgssg akgesgdpgp qgprgvqgpp gptgkpgkrg rpgadggrgm pgepgakgdr |
| 601 | gfdglpglpg dkghrgergp qgppgppgdd gmrgedgeig prglpgeagp rgllgprgtp |
| 661 | gapgqpgmag vdgppgpkgn mgpqgepgpp gqqgnpgpqg lpgpqgpigp pgekgpqgkp |
| 721 | glaglpgadg ppghpgkegq sgekgalgpp gpqgpigypg prgvkgadgv rglkgskgek |
| 781 | gedgfpgfkg dmglkgdrge vgqigprged gpegpkgrag ptgdpgpsgq agekgklgvp |
| 841 | glpgypgrqg pkgstgfpgf pgangekgar gvagkpgprg qrgptgprgs rgargptgkp |
| 901 | gpkgtsggdg ppgppgergp qgpqgpvgfp gpkgppgppg kdglpghpgq rgetgfqgkt |
| 961 | gppgpggvvg pqgptgetgp igerghpgpp gppgeqglpg aagkegakgd pgpqgisgkd |
| 1021 | gpaglrgfpg erglpgaqga pglkggegpq gppgpvgspg ergsagtagp iglpgrpgpq |
| 1081 | gppgpagekg apgekgpqgp agrdgvqgpv glpgpagpag spgedgdkge igepgqkgsk |
| 1141 | gdkgengppg ppglqgpvga pgiaggdgep gprgqqgmfg qkgdegargf pgppgpiglq |
| 1201 | glpgppgekg engdvgpmgp pgppgprgpq gpngadgpqg ppgsvgsvgg vgekgepgea |
| 1261 | gnpgppgeag vggpkgerge kgeagppgaa gppgakgppg ddgpkgnpgp vgfpgdpgpp |
| 1321 | gepgpagqdg vggdkgedgd pgqpgppgps geagppgppg krgppgaaga egrqgekgak |
| 1381 | geagaegppg ktgpvgpqgp agkpgpeglr gipgpvgeqg lpgaagqdgp pgpmgppglp |
| 1441 | glkgdpgskg ekghpgligl igppgeqgek gdrglpgtqg spgakgdggi pgpagplgpp |
| 1501 | gppglpgpqg pkgnkgstgp agqkgdsglp gppgspgppg eviqplpils skktrrhteg |
| 1561 | mqadaddnil dysdgmeeif gslnslkqdi ehmkfpmgtq tnpartckdl qlshpdfpdg |
| 1621 | eywidpnqgc sgdsfkvycn ftsggetciy pdkksegvri sswpkekpgs wfsefkrgkl |
| 1681 | lsyldvegns inmvqmtflk lltasarqnf tyhchqsaaw ydvssgsydk alrflgsnde |
| 1741 | emsydnnpfi ktlydgcasr kgyektviei ntpkidqvpi vdvmindfgd qnqkfgfevg |
| 1801 | pvcflg |

collagen alpha-1(XI) chain isoform C preproprotein [Homo sapiens] NP_542197.3
This variant (C, previously referred to as D) includes alternate exon
6A but lacks two other alternate exons, resulting in the loss of an
in-frame segment in the coding region, compared to variant A.
The encoded isoform (C, previously referred to as D) is shorter than isoform A.
(SEQ ID NO: 3)

| | |
|---|---|
| 1 | mepwssrwkt krwlwdftvt tlaltflfqa revrgaapvd vlkaldfhns pegiskttgf |
| 61 | ctnrknskgs dtayrvskqa qlsaptkqlf pggtfpedfs ilftvkpkkg iqsfllsiyn |
| 121 | ehgiqqigve vgrspvflfe dhtgkpaped yplfrtvnia dgkwhrvais vekktvtmiv |
| 181 | dckkkttkpl drseraivdt ngitvfgtri ldeevfegdi qqflitgdpk aaydycehys |
| 241 | pdcdssapka aqaqepqide yapediieyd yeygeaeyke aesvtegptv teetiaqtei |
| 301 | nghgaygekg qkgepavvep gmlvegppgp agpagimgpp glqgptgppg dpgdrgppgr |
| 361 | pglpgadglp gppgtadmlp fryggdgskg ptisaqeaqa qailqqaria lrgppgpmgl |
| 421 | tgrpgpvggp gssgakgesg dpgpqgprgv qgppgptgkp gkrgrpgadg grgmpgepga |
| 481 | kgdrgfdglp glpgdkghrg ergpqgppgp pgddgmrged geigprglpg eagprgllgp |
| 541 | rgtpgapgqp gmagvdgppg pkgnmgpqge pgppgqqgnp gpqglpgpqg pigppgekgp |
| 601 | qgkpglaglp gadgppghpg kegqsgekga lgppgpqgpi gypgprgvkg adgvrglkgs |
| 661 | kgekgedgfp gfkgdmglkg drgevgqigp rgedgpegpk gragptgdpg psgqagekgk |
| 721 | lgvpglpgyp grqgpkgstg fpgfpgange kgargvagkp gprgqrgptg prgsrgargp |
| 781 | tgkpgpkgts ggdgppgppg ergpqgpqgp vgfpgpkgpp gppgkdglpg hpgqrgetgf |
| 841 | qgktgppgpg gvvgpqgptg etgpigergh pgppgppgeq glpgaagkeg akgdpgpqgi |
| 901 | sgkdgpaglr gfpgerglpg aqgapglkgg egpqgppgpv gspgergsag tagpiglpgr |
| 961 | pgpqgppgpa gekgapgekg pqgpagrdgv qgpvglpgpa gpagspgedg dkgeigepgq |
| 1021 | kgskgdkgen gppgppglqg pvgapgiagg dgepgprgqq gmfgqkgdeg argfpgppgp |
| 1081 | iglqglpgpp gekgengdvg pmgppgppgp rgpqgpngad gpqgppgsvg svggvgekge |
| 1141 | pgeagnpgpp geagvggpkg ergekgeagp pgaagppgak gppgddgpkg npgpvgfpgd |
| 1201 | pgppgepgpa gqdgvggdkg edgdpgqpgp pgpsgeagpp gppgkrgppg aagaegrqge |
| 1261 | kgakgeagae gppgktgpvg pqgpagkpgp eglrgipgpv geqgipgaag qdgppgpmgp |
| 1321 | pglpglkgdp gskgekghpg ligligppge qgekgdrglp gtqgspgakg dggipgpagp |
| 1381 | igppgppglp gpqgpkgnkg stgpagqkgd sglpgppgsp gppgeviqpl pilsskktrr |
| 1441 | htegmqaaad dnildysdgm eeifgslnsl kqdiehmkfp mgtqtnpart ckdlqlshpd |
| 1501 | fpdgeywidp nqgcsgdsfk vycnftsgge tciypakkse gvrisswpke kpgswfsetk |
| 1561 | rgkllsyldv egnsinmvqm tfiklltasa rqnftyhchq saawydvssg sydkalrflg |
| 1621 | sndeemsydn npfiktlydg casrkgyekt vieintpkid qvpivdvmin dfgdqnqkfg |
| 1681 | fevgpvcfla |

Homo sapiens collagen type XI alpha 1 (COL11A1), transcript variant B, NM_080629.2
(SEQ ID NO: 5)

| | |
|---|---|
| 1 | acacagtact ctcagcttgt tggtggaagc ccctcatctg ccttcattct gaaggcaggg |
| 61 | cccggcagag gaaggatcag agggtcgcgg ccggagggtc ccggccggtg gggccaactc |
| 121 | agagggagag gaaagggcta gagacacgaa gaacgcaaac catcaaattt agaagaaaaa |
| 181 | gccctttgac tttttccccc tctccctccc caatggctgt gtagcaaaca tccctggcga |
| 241 | taccttggaa aggacgaagt tggtctgcag tcgcaatttc gtgggttgag ttcacagttg |
| 301 | tgagtgcggg gctcggagat ggagccgtgg tcctctaggt ggaaaacgaa acggtggctc |
| 361 | tgggatttca ccgtaacaac cctcgcattg accttcctct tccaagctag agaggtcaga |
| 421 | ggagctgctc cagttgatgt actaaaagca ctagattttc acaattctcc agagggaata |
| 481 | tcaaaaacaa cgggattttg cacaaacaga aagaattcta aaggctcaga tactgcttac |
| 541 | agagtttcaa agcaagcaca actcagtgcc ccaacaaaac agttatttcc aggtggaact |
| 601 | ttcccagaag acttttcaat actatttaca gtaaaaccaa aaaaaggaat tcagtctttc |
| 661 | cttttatcta tatataatga gcatggtatt cagcaaattg gtgttgaggt tgggagatca |
| 721 | cctgtttttc tgtttgaaga ccacactgga aaacctgccc cagaagacta tcccctcttc |
| 781 | agaactgtta acatcgctga cgggaagtgg catcgggtag caatcagcgt ggagaagaaa |
| 841 | actgtgacaa tgattgttga ttgtaagaag aaaaccacga aaccactcga tagaagtgag |
| 901 | agagcaattg tcgataccaa tggaatcacg gcttctggaa caaggatttt ggatgaagaa |
| 961 | gtttttgagg gggacattca gcagtttttg atcacaggtg atcccaaggc agcatatgac |
| 1021 | tactgtgagc attatagtcc agactgtgac tcttcagcac ccaaggctgc tcaagctcag |
| 1081 | gaacctcaga tagatgagaa aaagaaatcc aatttcaaaa agaagatgag gacagtggct |
| 1141 | actaaatcaa aggaaaaatc caaaaagttt acacccccca aatctgaaaa attttcatcc |
| 1201 | aagaagaaga aaagttatca agcatcagca aaagccaaac taggggtaaa ggcaaacatc |
| 1261 | gttgatgatt ttcaagaata caactatgga acaatggaaa gttaccagac agaagctcct |
| 1321 | aggcatgttt ctgggacaaa tgagccaaat ccagttgaag aaatatttac tgaagaatat |
| 1381 | ctaacgggag aggattatga ttcccagagg aaaaattctg aggatacact atatgaaaac |
| 1441 | aaagaaatag acggcaggga ttctgatctt ctggtagatg gagatttagg cgaatatgat |
| 1501 | ttttatgaat ataaagaata tgaagataaa ccaacaagcc cccctaatga agaatttggt |
| 1561 | ccaggtgtac cagcagaaac tgatattaca gaaacaagca taaatggcca tggtgcatat |
| 1621 | ggagagaaag gacagaaagg agaaccagca gtggttgagc ctggtatgct tgtcgaagga |
| 1681 | ccaccaggac cagcaggacc tgcaggtatt atgggtcctc caggtctaca aggccccact |
| 1741 | ggaccccctg gtgaccctgg cgataggggc cccccaggac gtcctggctt accaggggct |
| 1801 | gatggtctac ctggtcctcc tggtactatg ttgatgttac cgttccgcta tggtggtgat |
| 1861 | ggtcccaaag gaccaaccat ctctgctcag gaagctcagg ctcaagctat tcttcagcag |
| 1921 | gctcggattg ctctgagagg cccacctggc ccaatgggtc taactggaag accaggtcct |
| 1981 | gtgggggggc ctggttcatc tggggccaaa ggtgagagtg gtgatccagg tcctcagggc |
| 2041 | cctcgaggcg tccagggtcc ccctggtcca acgggaaaac ctggaaaaag gggtcgtcca |
| 2101 | ggtgcagatg gaggaagagg aatgccagga gaacctgggg caaagggaga tcgagggttt |
| 2161 | gatggacttc cgggtctgcc aggtgacaaa ggtcacaggg gtgaacgagg tcctcaaggt |
| 2221 | cctccaggtc ctcctggtga tgatggaatg aggggagaag atggagaaat tggaccaaga |
| 2281 | ggtcttccag gtgaagctgg cccacgaggt ttgctgggtc caaggggaac tccaggagct |
| 2341 | ccagggcagc ctggtatggc aggtgtagat ggccccccag gaccaaaagg gaacatgggt |
| 2401 | ccccaagggg agcctgggcc tccaggtcaa caagggaatc caggacctca gggtcttcct |
| 2461 | ggtccacaag gtccaattgg tcctcctggt gaaaaaggac cacaaggaaa accaggactt |
| 2521 | gctggacttc ctggtgctga tgggcctcct ggtcatcctg ggaaagaagg ccagtctgga |
| 2581 | gaaaaggggg ctctgggtcc ccctggtcca caaggtccta ttggataccc gggcccccgg |
| 2641 | ggagtaaagg gagcagatgg tgtcagaggt ctcaagggat ctaaaggtga aaagggtgaa |
| 2701 | gatggttttc caggattcaa aggtgacatg ggtctaaaag gtgacagagg agaagttggt |
| 2761 | caaattggcc caagagggga agatggccct gaaggaccca aaggtcgagc aggcccaact |
| 2821 | ggagacccag gtccttcagg tcaagcagga gaaaagggaa aacttggagt tccaggatta |
| 2881 | ccaggatatc caggaagaca aggtccaaag ggttccactg gattccctgg gtttccaggt |
| 2941 | gccaatggag agaaaggtgc acggggagta gctggcaaac caggccctcg gggtcagcgt |
| 3001 | ggtccaacgg gtcctcgagg ttcaagaggt gcaagaggtc ccactgggaa acctgggcca |
| 3061 | aagggcactt caggtggcga tggccctcct ggccctccag gtgaaagagg tcctcaagga |
| 3121 | cctcagggtc cagttggact ccctggacca aaaggccctc ctggaccacc tgggaaggat |
| 3181 | gggctgccag gacaccctgg gcaacgtggg gagactggat ttcaaggcaa gaccggccct |
| 3241 | cctgggccag ggggagtggt tggaccacag ggaccaaccg gtgagactgg tccaataggg |
| 3301 | gaacgtgggc atcctggccc tcctggccct cctggtgagc aaggtcttcc tggtgctgca |
| 3361 | ggaaaagaag gtgcaaaggg tgatccaggt cctcaaggta tctcagggaa agatggacca |
| 3421 | gcaggattac gtggtttccc aggggaaaga ggtcttcctg gagctcaggg tgcacctgga |
| 3481 | ctgaaaggag gggaaggtcc ccagggccca ccaggtccag ttggctcacc aggagaacgt |
| 3541 | gggtcagcag gtacagctgg cccaattggt ttaccagggc gcccgggacc tcagggtcct |
| 3601 | cctggtccag ctggagagaa aggtgctcct ggagaaaaag gtccccaagg gcctgcaggg |
| 3661 | agagatggag ttcaaggtcc tgttggtctc ccagggccag ctggtcctgc cggctcccct |
| 3721 | ggggaagacg gagacaaggg tgaaattggt gagccgggac aaaaaggcag caagggtgac |
| 3781 | aagggagaaa atggccctcc cggtccccca ggtcttcaag gaccagttgg tgcccctgga |
| 3841 | attgctggag gtgatggtga accaggtcct agaggacagc aggggatgtt tgggcaaaaa |
| 3901 | ggtgatgagg gtgccagagg cttccctgga cctcctggtc caataggtct tcagggtctg |
| 3961 | ccaggcccac ctggtgaaaa aggtgaaaat ggggatgttg gtcccatggg gccacctggt |
| 4021 | cctccaggcc caagaggccc tcaaggtccc aatggagctg atggaccaca aggaccccca |
| 4081 | gggtctgttg gtccagttgg tggtgttgga gaaaagggtg aacctggaga agcagggaac |
| 4141 | ccagggcctc ctggggaagc aggtgcaggc ggtcccaaag gagaaagagg agagaaaggg |
| 4201 | gaagctggtc cacctggagc tgctggacct ccaggtgcca aggggccacc aggtgatgat |
| 4261 | ggccctaagg gtaacccggg tcctgttggt tttcctggag atcctggtcc tcctggggaa |
| 4321 | cctggccctg caggtcaaga tggtgttggt ggtgacaagg gtgaagatgg agatcctggt |
| 4381 | caaccgggtc ctcctggccc atctggtgag gctggcccac caggtcctcc tggaaaacga |
| 4441 | ggtcctcctg gagctgcagg tgcagaggga agacaaggtg aaaaaggtgc taagggggaa |
| 4501 | gcaggtgcag aaggtcctcc tggaaaaacc ggcccagtcg gtcctcaggg acctgcagga |
| 4561 | aagcctggtc cagaaggtct tcggggcatc cctggtcctg tgggagaaca aggtctccct |
| 4621 | ggagctgcag gccaagatgg accacctggt cctatgggac ctcctggctt acctggtctc |
| 4681 | aaaggtgacc ctggctccaa gggtgaaaag ggacatcctg gtttaattgg cctgattggt |
| 4741 | cctccaggag aacaagggga aaaaggtgac cgagggctcc ctggaactca aggatctcca |
| 4801 | ggagcaaaag gggatggggg aattcctggt cctgctggtc ccttaggtcc acctggtcct |
| 4861 | ccaggtttac caggtcctca aggcccaaag ggtaacaaag gctctactgg acccgctggc |
| 4921 | cagaaaggtg acagtggtct tccagggcct cctgggtctc caggtccacc tggtgaagtc |
| 4981 | attcagcctt taccaatctt gtcctccaaa aaaacgagaa gacatactga aggcatgcaa |
| 5041 | gcagatgcag atgataatat tcttgattac tcggatggaa tggaagaaat atttggttcc |
| 5101 | ctcaattccc tgaaacaaga cattgagcat atgaaatttc caatgggtac tcagaccaat |
| 5161 | ccagcccgaa cttgtaaaga cctgcaactc agccatcctg acttcccaga tggtgaatat |
| 5221 | tggattgatc ctaaccaagg ttgctcagga gattccttca aagtttactg taatttcaca |
| 5281 | tctggtggtg agacttgcat ttatccagac aaaaaatctg agggagtaag aatttcatca |
| 5341 | tggccaaagg agaaaccagg aagttggttt agtgaattta agaggggaaa actgctttca |
| 5401 | tacttagatg ttgaaggaaa ttccatcaat atggtgcaaa tgacattcct gaaacttctg |
| 5461 | actgcctctg ctcggcaaaa tttcacctac cactgtcatc agtcagcagc ctggtatgat |
| 5521 | gtgtcatcag gaagttatga caaagcactt cgcttcctgg gatcaaatga tgaggagatg |
| 5581 | tcctatgaca ataatccttt tatcaaaaca ctgtatgatg gttgtgcgtc cagaaaaggc |
| 5641 | tatgaaaaga ctgtcattga aatcaataca ccaaaaattg atcaagtacc tattgttgat |
| 5701 | gtcatgatca atgactttgg tgatcagaat cagaagttcg gatttgaagt tggtcctgtt |
| 5761 | tgttttcttg gctaagatta agacaaagaa catatcaaat caacagaaaa tataccttgg |
| 5821 | tgccaccaac ccattttgtg ccacatgcaa gttttgaata aggatggtat agaaaacaac |
| 5881 | gctgcatata caggtaccat ttaggaaata ccgatgcctt tgtgggggca gaatcacatg |
| 5941 | gcaaaagctt tgaaaatcat aaagatataa gttggtgtgg ctaagatgga aacagggctg |
| 6001 | attcttgatt cccaattctc aactctcctt ttcctatttg aatttctttg gtgctgtaga |
| 6061 | aaacaaaaaa agaaaaatat atattcataa aaaatatggt gctcattctc atccatccag |
| 6121 | gatgtactaa aacagtgtgt ttaataaatt gtaattattt tgtgtacagt tctatactgt |
| 6181 | tatctgtgtc catttccaaa acttgcacgt gtccctgaat tccatctgac tctaatttta |
| 6241 | tgagaattgc agaactctga tggcaataaa tatatgtatt atgaaaaaat aaagttgtaa |
| 6301 | tttctgatga ctctaagtcc ctttctttgg ttaataataa aatgcctttg tatatattga |
| 6361 | tgttgaagag ttcaattatt tgatgtcgcc aacaaaattc tcagagggca aaaatctgga |
| 6421 | agacttttgg aagcacactc tgatcaactc ttctctgccg acagtcattt tgctgaattt |
| 6481 | cagccaaaaa tattatgcat tttgatgctt tattcaaggc tatacctcaa actttttctt |
| 6541 | ctcagaatcc aggatttcac aggatacttg tatatatgga aaacaagcaa gtttatattt |
| 6601 | ttggacaggg aaatgtgtgt aagaaagtat attaacaaat caatgcctcc gtcaagcaaa |
| 6661 | caatcatatg tatacttttt ttctacgtta tctcatctcc ttgttttcag tgtgcttcaa |
| 6721 | taatgcaggt taatattaaa gatggaaatt aagcaattat ttatgaattt gtgcaatgtt |
| 6781 | agattttctt atcaatcaag ttcttgaatt tgattctaag ttgcatatta taacagtctc |
| 6841 | gaaaattatt ttacttgccc aacaaatatt acttttttcc tttcaagata attttataaa |
| 6901 | tcatttgacc tacctaattg ctaaatgaat aacatatggt ggactgttat taagagtatt |
| 6961 | tgttttaagt cattcaggaa aatctaaact tttttttcca ctaaggtatt tactttaagg |
| 7021 | tagcttgaaa tagcaataca atttaaaaat taaaaactga attttgtatc tattttaagt |
| 7081 | aatatatgta agacttgaaa ataaatgttt tatttcttat ataaagtgtt aaattaattg |
| 7141 | ataccagatt tcactggaac agtttcaact gataatttat gacaaaagaa catacctgta |
| 7201 | atattgaaat taaaaagtga aatttgtcat aaagaatttc ttttattttt gaaatcgagt |
| 7261 | ttgtaaatgt ccttttaaga agggagatat gaatccaata aataaactca agtcttggct |
| 7321 | acctgga |

[symbol]

indicates data missing or illegible when filed

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

TABLE OF SEQUENCES

| | |
|---|---|
| SEQ ID NO: 1 | Col 11A1 fragment |
| SEQ ID NO: 2 | Col 11A1 amino acid sequence |
| SEQ ID NO: 3 | Col 11A1 amino acid sequence |
| SEQ ID NO: 4 | Col 11A1 fragment DNA sequence |
| SEQ ID NO: 5 | Col 11A1 DNA sequence |
| SEQ ID NO: 6 | Col 11A1 DNA sequence |
| SEQ ID NO: 7 | ALP Sense Primer |
| SEQ ID NO: 8 | ALP Antisense Primer |
| SEQ ID NO: 9 | Col 1A1 Sense Primer |
| SEQ ID NO: 10 | Col 1A1 Antisense Primer |
| SEQ ID NO: 11 | Col 11A1 e6A Sense Primer |
| SEQ ID NO: 12 | Col 11A1 e6A Antisense Primer |
| SEQ ID NO: 13 | Col 11A1 e6B Sense Primer |
| SEQ ID NO: 14 | Col 11A1 e6B Antisense Primer |
| SEQ ID NO: 15 | Col 11A1 e7 Sense Primer |
| SEQ ID NO: 16 | Col 11A1 e7 Antisense Primer |
| SEQ ID NO: 17 | Col 11A1 e8 Sense Primer |
| SEQ ID NO: 18 | Col 11A1 e8 Antisense Primer |
| SEQ ID NO: 19 | Osteocalcin Sense Primer |
| SEQ ID NO: 20 | Osteocalcin Antisense Primer |
| SEQ ID NO: 21 | PPIA Sense Primer |
| SEQ ID NO: 22 | PPIA Antisense Primer |
| SEQ ID NO: 23 | RUNX2 Sense Primer |
| SEQ ID NO: 24 | RUNX2 Antisense Primer |

What is claimed is:

1. A method of modulating bone matrix mineralization in a subject, said method comprising administering to said subject a therapeutically effective amount of a pharmaceutical composition comprising:
(a) a Col 11A1 protein or inhibitor thereof; and
(b) a pharmaceutically acceptable carrier.

2. The method of claim 1, wherein (a) said Col 11A1 protein is a Col 11A1 fragment.

3. The method of claim 1 wherein said Col 11A1 protein is one or more of the following:
(a) the amino acid sequence of SEQ ID NOS: 1, 2, or 3;
(b) the amino acid sequence at least 85% sequence identity to SEQ ID NO: 1, 2, or 3;
(c) a conservatively modified variant of SEQ ID NOS: 1, 2, or 3;
(d) a fragment of 1,2, or 3 wherein said fragment modulation bone mineralization.

4. The method of claim 2, wherein said fragment is SEQ ID NO:1.

5. The method of claim 1, wherein said carrier is saline.

6. The method of claim 1, wherein (a) said polypeptide is pegylated, (b) said polypeptide is glycosylated, (c) said pharmaceutical composition comprises a dimer of said polypeptide, (d) said pharmaceutically acceptable excipient comprises saline, or (e) said pharmaceutical composition is lyophilized.

7. The method of claim 1, wherein said pharmaceutical composition is administered subcutaneously, intravenously, orally, nasally, intramuscularly, sublingually, intrathecally, or intradermally.

8. The method of claim 1 wherein said subject in need of bone mineralization is a subject suffering from a bone fracture.

9. The method of claim 1 wherein said subject is suffering from a bone mineralization disease.

10. The method of claim 9, wherein said bone mineralization disorder is hypophosphatasia, optionally wherein said matrix mineralization disorder is infantile hypophosphatasia (HPP), childhood HPP, perinatal HPP, adult HPP, or odontohypophosphatasia.

11. The method of claim 8 wherein said subject is suffering from osteoporosis.

12. The method of claim 1, wherein said subject is human.

13. A pharmaceutical composition comprising:
(a) a Col 11A1 polypeptide; and
(b) a pharmaceutically acceptable carrier.

14. A method of modulating bone mineralization in a subject, said method comprising administering to said subject a therapeutically effective amount of a pharmaceutical composition comprising:
(a) an isolated nucleic acid molecule encoding a Col 11A1 polypeptide; and
(b) a pharmaceutically acceptable carrier.

15. The method of claim 14 wherein said pharmaceutical composition is an expression vector.

16. The method of claim 15 wherein said expression vector is a lentiviral vector.

17. An isolated recombinant host cell transformed or transfected with a lentiviral recombinant expression vector comprising the isolated nucleic acid molecule of claim 14.

18. The method of claim 14 wherein said expression vector is an inhibition construct.

19. The method of claim 18 wherein said inhibition construct is an antisense oligonucleotide.

20. The method of claim 19 wherein said antisense oligonucleotide is a morpholino oligonucleotide.

21. A pharmaceutical composition comprising:
(a) an isolated nucleic acid molecule encoding a Col 11A1 polypeptide; and
(b) a pharmaceutically acceptable carrier.

22. The pharmaceutical composition of claim 21 wherein said nucleic acid is an expression vector.

23. The pharmaceutical composting of claim 22 wherein said expression vector is a lentiviral vector.

24. The pharmaceutical composition of claim 21 wherein said expression vector is an inhibition construct.

25. The pharmaceutical composition of claim 24 wherein said inhibition construct is an antisense oligonucleotide.

26. The pharmaceutical composition of claim 25 wherein said antisense oligonucleotide is a morpholino oligonucleotide.

27. An Col 11a1 fragment wherein the Col 11A1 fragment comprises SEQ ID NO:1 and is less that the full length Col 11A1 sequence of SEQ IN NO: 1 or 2.

28. A nucleic acid sequence encoding the Col 11A1 fragment of claim 27.

29. The isolated nucleic acid molecule of claim 28 operably linked to a promoter.

30. An expression vector comprising the isolated nucleic acid molecule of claim 28.

31. The expression vector of claim 30, wherein the expression vector comprises a promoter, wherein the promoter is a cytomegalovirus promoter.

32. The expression vector of claim 31, further encoding a selectable marker.

33. The expression vector of claim 31, wherein the expression vector comprises a mammalian expression vector.

34. The expression vector of claim 30, wherein the mammalian expression vector comprises a viral expression vector.

35. A viral particle comprising the expression vector of claim 33.


SEQUENCE LISTING (24 sequences)

SEQ ID NO: 1 - protein, 327 aa, Homo sapiens
ASPVDILKALDFHNSPVGISKTTGFCTSRKNSKDPDIAYRVTEEAQISAPTKQLFPGGIFPQDFSILFTIKPKKGTQAFLLSLYNEHGIQQLGVEVGRSPVFLFEDHTGKPTPENYPLFSTVNIADGKWHRVAISVEKKTVTMIVDCKKKITKPLDRSERSIVDTNGIMVFGTRILETDVFQGDIQQFLITGDPKAAYDYCDHYSPDCDLTSPKAAQAQEPHIDEKKKSNYTKKKRTLATNSKKKSKMSTTPKSEKFASKKKKRNQATAKAKLGVQANIVDDFQDYNYGTMETYQTESPRRVSGSNEINGHGAYGEKGQKGEPAVVE

SEQ ID NO: 2 - protein, 1818 aa, Homo sapiens
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEKKKSNFKKKMRTVATKSKEKSKKFTPPKSEKFSSKKKKSYQASAKAKLGVKANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGYPGPRGVKGADGVRGLKGSKGEKGEDGFPGFKGDMGLKGDRGEVGQIGPRGEDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGKDGLPGHPGQRGETGFQGKTGPPGPGGVVGPQGPTGETGPIGERGHPGPPGPPGEQGLPGAAGKEGAKGDPGPQGISGKDGPAGLRGFPGERGLPGAQGAPGLKGGEGPQGPPGPVGSPGERGSAGTAGPIGLPGRPGPQGPPGPAGEKGAPGEKGPQGPAGRDGVQGPVGLPGPAGPAGSPGEDGDKGEIGEPGQKGSKGDKGENGPPGPPGLQGPVGAPGIAGGDGEPGPRGQQGMFGQKGDEGARGFPGPPGPIGLQGLPGPPGEKGENGDVGPMGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGEPGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGSPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCASRKGYEKTVIEINTPKIDQVPIVDVMINDFGDQNQKFGFEVGPVCFLG

SEQ ID NO: 3 - protein, 1818 aa, Homo sapiens [sequence omitted]

SEQ ID NO: 4 - DNA, 5454 bp, Homo sapiens
atggagccctggagcagcaggtggaagaccaagaggtggctgtgggacttcaccgtgaccaccctggccctgaccttcctgttccaggccagggaggtgaggggcgccgcccccgtggacgtgctgaaggccctggacttccacaacagccccgagggcatcagcaagaccaccggcttctgcaccaacaggaagaacagcaagggcagcgacaccgcctacagggtgagcaagcaggcccagctgagcgcccccaccaagcagctgttccccggcggcaccttccccgaggacttcagcatcctgttcaccgtgaagcccaagaagggcatccagagcttcctgctgagcatctacaacgagcacggcatccagcagatcggcgtggaggtgggcaggagccccgtgttcctgttcgaggaccacaccggcaagcccgcccccgaggactaccccctgttcaggaccgtgaacatcgccgacggcaagtggcacagggtggccatcagcgtggagaagaagaccgtgaccatgatcgtggactgcaagaagaagaccaccaagcccctggacaggagcgagagggccatcgtggacaccaacggcatcaccgtgttcggcaccaggatcctggacgaggaggtgttcgagggcgacatccagcagttcctgatcaccggcgaccccaaggccgcctacgactactgcgagcactacagccccgactgcgacagcagcgcccccaaggccgcccaggcccaggagccccagatcgacgagaagaagaagagcaacttcaagaagaagatgaggaccgtggccaccaagagcaaggagaagagcaagaagttcaccccccccaagagcgagaagttcagcagcaagaagaagaagagctaccaggccagcgccaaggccaagctgggcgtgaaggccaacatcgtggacgacttccaggagtacaactacggcaccatggagagctaccagaccgaggcccccaggcacgtgagcggcaccaacgagcccaaccccgtggaggagatcttcaccgaggagtacctgaccggcgaggactacgacagccagaggaagaacagcgaggacaccctgtacgagaacaaggagatcgacggcagggacagcgacctgctggtggacggcgacctgggcgagtacgacttctacgagtacaaggagtacgaggacaagcccaccagcccccccaacgaggagttcggccccggcgtgcccgccgagaccgacatcaccgagaccagcatcaacggccacggcgcctacggcgagaagggccagaagggcgagcccgccgtggtggagcccggcatgctggtggagggcccccccggccccgccggccccgccggcatcatgggcccccccggcctgcagggccccaccggcccccccggcgaccccggcgacaggggcccccccggcaggcccggcctgcccggcgccgacggcctgcccggcccccccggcaccatgctgatgctgcccttcaggtacggcggcgacggcagcaagggccccaccatcagcgcccaggaggcccaggcccaggccatcctgcagcaggccaggatcgccctgaggggcccccccggccccatgggcctgaccggcaggcccggccccgtgggcggccccggcagcagcggcgccaagggcgagagcggcgaccccggcccccagggccccaggggcgtgcagggcccccccggccccaccggcaagcccggcaagaggggcaggcccggcgccgacggcggcaggggcatgcccggcgagcccggcgccaagggcgacaggggcttcgacggcctgcccggcctgcccggcgacaagggccacaggggcgagaggggcccccagggcccccccggcccccccggcgacgacggcatgaggggcgaggacggcgagatcggccccaggggcctgcccggcgaggccggccccaggggcctgctgggccccaggggcacccccggcgcccccggccagcccggcatggccggcgtggacggcccccccggccccaagggcaacatgggcccccagggcgagcccggcccccccggccagcagggcaaccccggcccccagggcctgcccggcccccagggccccatcggcccccccggcgagaagggcccccagggcaagcccggcctggccggcctgcccggcgccgacggcccccccggccaccccggcaaggagggccagagcggcgagaagggcgccctgggcccccccggcccccagggccccatcggctaccccggccccaggggcgtgaagggcgccgacggcgtgaggggcctgaagggcagcaagggcgagaagggcgaggacggcttccccggcttcaagggcgacatgggcctgaagggcgacaggggcgaggtgggccagatcggccccaggggcgaggacggccccgagggccccaagggcagggccggccccaccggcgaccccggccccagcggccaggccggcgagaagggcaagctgggcgtgcccggcctgcccggctaccccggcaggcagggccccaagggcagcaccggcttccccggcttccccggcgccaacggcgagaagggcgccaggggcgtggccggcaagcccggccccaggggccagaggggccccaccggccccaggggcagcaggggcgccaggggccccaccggcaagcccggccccaagggcaccagcggcggcgacggcccccccggcccccccggcgagaggggcccccagggcccccagggccccgtgggcttccccggccccaagggcccccccggcccccccggcaaggacggcctgcccggccaccccggccagaggggcgagaccggcttccagggcaagaccggcccccccggccccggcggcgtggtgggcccccagggccccaccggcgagaccggccccatcggcgagaggggccaccccggcccccccggcccccccggcgagcagggcctgcccggcgccgccggcaaggagggcgccaagggcgaccccggcccccagggcatcagcggcaaggacggccccgccggcctgaggggcttccccggcgagaggggcctgcccggcgcccagggcgcccccggcctgaagggcggcgagggcccccagggcccccccggccccgtgggcagccccggcgagaggggcagcgccggcaccgccggccccatcggcctgcccggcaggcccggcccccagggcccccccggccccgccggcgagaagggcgcccccggcgagaagggcccccagggccccgccggcagggacggcgtgcagggccccgtgggcctgcccggccccgccggccccgccggcagccccggcgaggacggcgacaagggcgagatcggcgagcccggccagaagggcagcaagggcgacaagggcgagaacggcccccccggcccccccggcctgcagggccccgtgggcgcccccggcatcgccggcggcgacggcgagcccggccccaggggccagcagggcatgttcggccagaagggcgacgagggcgccaggggcttccccggcccccccggccccatcggcctgcagggcctgcccggcccccccggcgagaagggcgagaacggcgacgtgggccccatgggcccccccggcccccccggccccaggggcccccagggccccaacggcgccgacggcccccagggcccccccggcagcgtgggcagcgtgggcggcgtgggcgagaagggcgagcccggcgaggccggcaaccccggcccccccggcgaggccggcgtgggcggccccaagggcgagaggggcgagaagggcgaggccggcccccccggcgccgccggcccccccggcgccaagggcccccccggcgacgacggccccaagggcaaccccggccccgtgggcttccccggcgaccccggcccccccggcgagcccggccccgccggccaggacggcgtgggcggcgacaagggcgaggacggcgaccccggccagcccggcccccccggccccagcggcgaggccggcccccccggcccccccggcaagaggggcccccccggcgccgccggcgccgagggcaggcagggcgagaagggcgccaagggcgaggccggcgccgagggcccccccggcaagaccggccccgtgggcccccagggccccgccggcaagcccggccccgagggcctgaggggcatccccggccccgtgggcgagcagggcctgcccggcgccgccggccaggacggcccccccggccccatgggcccccccggcctgcccggcctgaagggcgaccccggcagcaagggcgagaagggccaccccggcctgatcggcctgatcggcccccccggcgagcagggcgagaagggcgacaggggcctgcccggcacccagggcagccccggcgccaagggcgacggcggcatccccggccccgccggccccctgggcccccccggcccccccggcctgcccggcccccagggccccaagggcaacaagggcagcaccggccccgccggccagaagggcgacagcggcctgcccggcccccccggcagccccggcccccccggcgaggtgatccagcccctgcccatcctgagcagcaagaagaccaggaggcacaccgagggcatgcaggccgacgccgacgacaacatcctggactacagcgacggcatggaggagatcttcggcagcctgaacagcctgaagcaggacatcgagcacatgaagttccccatgggcacccagaccaaccccgccaggacctgcaaggacctgcagctgagccaccccgacttccccgacggcgagtactggatcgaccccaaccagggctgcagcggcgacagcttcaaggtgtactgcaacttcaccagcggcggcgagacctgcatctaccccgacaagaagagcgagggcgtgaggatcagcagctggcccaaggagaagcccggcagctggttcagcgagttcaagaggggcaagctgctgagctacctggacgtggagggcaacagcatcaacatggtgcagatgaccttcctgaagctgctgaccgccagcgccaggcagaacttcacctaccactgccaccagagcgccgcctggtacgacgtgagcagcggcagctacgacaaggccctgaggttcctgggcagcaacgacgaggagatgagctacgacaacaaccccttcatcaagaccctgtacgacggctgcgccagcaggaagggctacgagaagaccgtgatcgagatcaacacccccaagatcgaccaggtgcccatcgtggacgtgatgatcaacgacttcggcgaccagaaccagaagttcggcttcgaggtgggccccgtgtgcttcctgggc

SEQ ID NO: 5 - DNA, 7320 bp, Homo sapiens
acacagtactctcagcttgttggtggaagcccctcatctgccttcattctgaaggcagggcccggcagaggaaggatcagagggtcgcggccggagggtcccggccggtggggccaactcagagggagaggaaagggctagagacacgaagaacgcaaaccatcaaatttagaagaaaaagccctttgactttttccccctctccctccccaatggctgtgtagcaaacatccctggcgataccttggaaaggacgaagttggtctgcagtcgcaatttcgtgggttgagttcacagttgtgagtgcggggctcggagatggagccgtggtcctctaggtggaaaacgaaacggtggctctgggatttcaccgtaacaaccctcgcattgaccttcctcttccaagctagagaggtcagaggagctgctccagttgatgtactaaaagcactagattttcacaattctccagagggaatatcaaaaacaacgggattttgcacaaacagaaagaattctaaaggctcagatactgcttacagagtttcaaagcaagcacaactcagtgccccaacaaaacagttatttccaggtggaactttcccagaagacttttcaatactatttacagtaaaaccaaaaaaaggaattcagtctttccttttatctatatataatgagcatggtattcagcaaattggtgttgaggttgggagatcacctgtttttctgtttgaagaccacactggaaaacctgccccagaagactatcccctcttcagaactgttaacatcgctgacgggaagtggcatcgggtagcaatcagcgtggagaagaaaactgtgacaatgattgttgattgtaagaagaaaaccacgaaaccacttgatagaagtgagagagcaattgttgataccaatggaatcacggtttttggaacaaggattttggatgaagaagtttttgagggggacattcagcagtttttgatcacaggtgatcccaaggcagcatatgactactgtgagcattatagtccagactgtgactcttcagcacccaaggctgctcaagctcaggaacctcagatagatgagaaaaagaaatccaatttcaaaaagaagatgaggacagtggctactaaatcaaaggaaaaatccaaaaagtttacaccccccaaatctgaaaaattttcatccaagaagaagaaaagttatcaagcatcagcaaaagccaaactaggggtaaaggcaaacatcgttgatgattttcaagaatacaactatggaacaatggaaagttaccagacagaagctcctaggcatgtttctgggacaaatgagccaaatccagttgaagaaatatttactgaagaatatctaacgggagaggattatgattcccagaggaaaaattctgaggatacactatatgaaaacaaagaaatagacggcagggattctgatcttctggtagatggagatttaggcgaatatgatttttatgaatataaagaatatgaagataaaccaacaagcccccctaatgaagaatttggtccaggtgtaccagcagaaactgatattacagaaacaagcataaatggccatggtgcatatggagagaaaggacagaaaggagaaccagcagtggttgagcctggtatgcttgtcgaaggaccaccaggaccagcaggacctgcaggtattatgggtcctccaggtctacaaggccccactggaccccctggtgaccctggcgataggggccccccaggacgtcctggcttaccaggggctgatggtctacctggtcctcctggtactatgttgatgttaccgttccgttatggtggtgatggttccaaaggaccaaccatctctgctcaggaagctcaggctcaagctattcttcagcaggctcggattgctctgagaggcccacctggcccaatgggtctaactggaagaccaggtcctgtgggggggcctggttcatctggggccaaaggtgagagtggtgatccaggtcctcagggccctcgaggcgtccagggtccccctggtccaacgggaaaacctggaaaaaggggtcgtccaggtgcagatggaggaagaggaatgccaggagaacctggggcaaagggagatcgagggtttgatggacttccgggtctgccaggtgacaaaggtcacaggggtgaacgaggtcctcaaggtcctccaggtcctcctggtgatgatggaatgaggggagaagatggagaaattggaccaagaggtcttccaggtgaagctggcccacgaggtttgctgggtccaaggggaactccaggagctccagggcagcctggtatggcaggtgtagatggccccccaggaccaaaagggaacatgggtccccaaggggagcctgggcctccaggtcaacaagggaatccaggacctcagggtcttcctggtccacaaggtccaattggtcctcctggtgaaaaaggaccacaaggaaaaccaggacttgctggacttcctggtgctgatgggcctcctggtcatcctgggaaagaaggccagtctggagaaaagggggctctgggtccccctggtccacaaggtcctattggatacccgggcccccggggagtaaagggagcagatggtgtcagaggtctcaagggatctaaaggtgaaaagggtgaagatggttttccaggattcaaaggtgacatgggtctaaaaggtgacagaggagaagttggtcaaattggcccaagaggggaagatggccctgaaggacccaaaggtcgagcaggcccaactggagacccaggtccttcaggtcaagcaggagaaaagggaaaacttggagttccaggattaccaggatatccaggaagacaaggtccaaagggttccactggattccctgggtttccaggtgccaatggagagaaaggtgcacggggagtagctggcaaaccaggccctcggggtcagcgtggtccaacgggtcctcgaggttcaagaggtgcaagaggtcccactgggaaacctgggccaaagggcacttcaggtggcgatggccctcctggccctccaggtgaaagaggtcctcaaggacctcagggtccagttggattccctggaccaaaaggccctcctggaccacctgggaaggatgggctgccaggacaccctgggcaacgtggggagactggatttcaaggcaagaccggccctcctgggccagggggagtggttggaccacagggaccaaccggtgagactggtccaataggggaacgtgggcatcctggccctcctggccctcctggtgagcaaggtcttcctggtgctgcaggaaaagaaggtgcaaagggtgatccaggtcctcaaggtatctcagggaaagatggaccagcaggattacgtggtttcccaggggaaagaggtcttcctggagctcagggtgcacctggactgaaaggaggggaaggtccccagggcccaccaggtccagttggctcaccaggagaacgtgggtcagcaggtacagctggcccaattggtttaccagggcgcccgggacctcagggtcctcctggtccagctggagagaaaggtgctcctggagaaaaaggtccccaagggcctgcagggagagatggagttcaaggtcctgttggtctcccagggccagctggtcctgccggctcccctggggaagacggagacaagggtgaaattggtgagccgggacaaaaaggcagcaagggtgacaagggagaaaatggccctcccggtcccccaggtcttcaaggaccagttggtgcccctggaattgctggaggtgatggtgaaccaggtcctagaggacagcaggggatgtttgggcaaaaaggtgatgagggtgccagaggcttccctggacctcctggtccaataggtcttcagggtctgccaggcccacctggtgaaaaaggtgaaaatggggatgttggtcccatggggccacctggtcctccaggcccaagaggccctcaaggtcccaatggagctgatggaccacaaggacccccagggtctgttggttcagttggtggtgttggagaaaagggtgaacctggagaagcagggaacccagggcctcctggggaagcaggtgtaggcggtcccaaaggagaaagaggagagaaaggggaagctggtccacctggagctgctggacctccaggtgccaaggggccaccaggtgatgatggccctaagggtaacccgggtcctgttggttttcctggagatcctggtcctcctggggaacctggccctgcaggtcaagatggtgttggtggtgacaagggtgaagatggagatcctggtcaaccgggtcctcctggcccatctggtgaggctggcccaccaggtcctcctggaaaacgaggtcctcctggagctgcaggtgcagagggaagacaaggtgaaaaaggtgctaagggggaagcaggtgcagaaggtcctcctggaaaaaccggcccagtcggtcctcagggacctgcaggaaagcctggtccagaaggtcttcggggcatccctggtcctgtgggagaacaaggtctccctggagctgcaggccaagatggaccacctggtcctatgggacctcctggcttacctggtctcaaaggtgaccctggctccaagggtgaaaagggacatcctggtttaattggcctgattggtcctccaggagaacaaggggaaaaaggtgaccgagggctccctggaactcaaggatctccaggagcaaaaggggatgggggaattcctggtcctgctggtcccttaggtccacctggtcctccaggtttaccaggtcctcaaggcccaaagggtaacaaaggctctactggacccgctggccagaaaggtgacagtggtcttccagggcctcctgggtctccaggtccacctggtgaagtcattcagcctttaccaatcttgtcctccaaaaaaacgagaagacatactgaaggcatgcaagcagatgcagatgataatattcttgattactcggatggaatggaagaaatatttggttccctcaattccctgaaacaagacattgagcatatgaaatttccaatgggtactcagaccaatccagcccgaacttgtaaagacctgcaactcagccatcctgacttcccagatggtgaatattggattgatcctaaccaaggttgctcaggagattccttcaaagtttactgtaatttcacatctggtggtgagacttgcatttatccagacaaaaaatctgagggagtaagaatttcatcatggccaaaggagaaaccaggaagttggtttagtgaatttaagaggggaaaactgctttcatacttagatgttgaaggaaattccatcaatatggtgcaaatgacattcctgaaacttctgactgcctctgctcggcaaaatttcacctaccactgtcatcagtcagcagcctggtatgatgtgtcatcaggaagttatgacaaagcacttcgcttcctgggatcaaatgatgaggagatgtcctatgacaataatccttttatcaaaacactgtatgatggttgtgcgtccagaaaaggctatgaaaagactgtcattgaaatcaatacaccaaaaattgatcaagtacctattgttgatgtcatgatcaatgactttggtgatcagaatcagaagttcggatttgaagttggtcctgtttgttttcttggctaagattaagacaaagaacatatcaaatcaacagaaaatataccttggtgccaccaacccattttgtgccacatgcaagttttgaataaggatggtatagaaaacaacgctgcatatacaggtaccatttaggaaataccgatgcctttgtgggggcagaatcacatggcaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattccatctgactctaattttatgagaattgcagaactctgatggcaataaatatatgtattatgaaaaaataaagttgtaatttctgatgactctaagtccctttctttggttaataataaaatgcctttgtatatattgatgttgaagagttcaattatttgatgtcgccaacaaaattctcagagggcaaaaatctggaagacttttggaagcacactctgatcaactcttctctgccgacagtcattttgctgaatttcagccaaaaatattatgcattttgatgctttattcaaggctatacctcaaactttttcttctcagaatccaggatttcacaggatacttgtatatatggaaaacaagcaagtttatatttttggacagggaaatgtgtgtaagaaagtatattaacaaatcaatgcctccgtcaagcaaacaatcatatgtatactttttttctacgttatctcatctccttgttttcagtgtgcttcaataatgcaggttaatattaaagatggaaattaagcaattatttatgaatttgtgcaatgttagattttcttatcaatcaagttcttgaatttgattctaagttgcatattataacagtctcgaaaattattttacttgcccaacaaatattacttttttcctttcaagataattttataaatcatttgacctacctaattgctaaatgaataacatatggtggactgttattaagagtatttgttttaagtcattcaggaaaatctaaacttttttttccactaaggtatttactttaaggtagcttgaaatagcaatacaatttaaaaattaaaaactgaattttgtatctattttaagtaatatatgtaagacttgaaaataaatgttttatttcttatataaagtgttaaattaattgataccagatttcactggaacagtttcaactgataatttatgacaaaagaacatacctgtaatattgaaattaaaaagtgaaatttgtcataaagaatttcttttatttttgaaatcgagtttgtaaatgtccttttaagaagggagatatgaatccaataaataaactcaagtcttggct

SEQ ID NO: 6 - DNA, 5454 bp, Homo sapiens
atggagccctggagcagcaggtggaagaccaagaggtggctgtgggacttcaccgtgaccaccctggccctgaccttcctgttccaggccagggaggtgaggggcgccgcccccgtggacgtgctgaaggccctggacttccacaacagccccgagggcatcagcaagaccaccggcttctgcaccaacaggaagaacagcaagggcagcgacaccgcctacagggtgagcaagcaggcccagctgagcgcccccaccaagcagctgttccccggcggcaccttccccgaggacttcagcatcctgttcaccgtgaagcccaagaagggcatccagagcttcctgctgagcatctacaacgagcacggcatccagcagatcggcgtggaggtgggcaggagccccgtgttcctgttcgaggaccacaccggcaagcccgcccccgaggactaccccctgttcaggaccgtgaacatcgccgacggcaagtggcacagggtggccatcagcgtggagaagaagaccgtgaccatgatcgtggactgcaagaagaagaccaccaagcccctggacaggagcgagagggccatcgtggacaccaacggcatcaccgtgttcggcaccaggatcctggacgaggaggtgttcgagggcgacatccagcagttcctgatcaccggcgaccccaaggccgcctacgactactgcgagcactacagccccgactgcgacagcagcgcccccaaggccgcccaggcccaggagccccagatcgacgagaagaagaagagcaacttcaagaagaagatgaggaccgtggccaccaacagcaaggagaagagcaagaagttcaccccccccaagagcgagaagttcagcagcaagaagaagaagagctaccaggccagcgccaaggccaagctgggcgtgaaggccaacatcgtggacgacttccaggagtacaactacggcaccatggagagctaccagaccgaggcccccaggcacgtgagcggcaccaacgagcccaaccccgtggaggagatcttcaccgaggagtacctgaccggcgaggactacgacagccagaggaagaacagcgaggacaccctgtacgagaacaaggagatcgacggcagggacagcgacctgctggtggacggcgacctgggcgagtacgacttctacgagtacaaggagtacgaggacaagcccaccagcccccccaacgaggagttcggccccggcgtgcccgccgagaccgacatcaccgagaccagcatcaacggccacggcgcctacggcgagaagggccagaagggcgagcccgccgtggtggagcccggcatgctggtggagggcccccccggccccgccggccccgccggcatcatgggcccccccggcctgcagggccccaccggcccccccggcgaccccggcgacaggggcccccccggcaggcccggcctgcccggcgccgacggcctgcccggcccccccggcaccatgctgatgctgcccttcaggtacggcggcgacggcagcaagggccccaccatcagcgcccaggaggcccaggcccaggccatcctgcagcaggccaggatcgccctgaggggcccccccggccccatgggcctgaccggcaggcccggccccgtgggcggccccggcagcagcggcgccaagggcgagagcggcgaccccggcccccagggccccaggggcgtgcagggcccccccggccccaccggcaagcccggcaagaggggcaggcccggcgccgacggcggcaggggcatgcccggcgagcccggcgccaagggcgacaggggcttcgacggcctgcccggcctgcccggcgacaagggccacaggggcgagaggggcccccagggcccccccggcccccccggcgacgacggcatgaggggcgaggacggcgagatcggccccaggggcctgcccggcgaggccggccccaggggcctgctgggccccaggggcacccccggcgcccccggccagcccggcatggccggcgtggacggcccccccggccccaagggcaacatgggcccccagggcgagcccggcccccccggccagcagggcaaccccggcccccagggcctgcccggcccccagggccccatcggcccccccggcgagaagggcccccagggcaagcccggcctggccggcctgcccggcgccgacggcccccccggccaccccggcaaggagggccagagcggcgagaagggcgccctgggcccccccggcccccagggccccatcggctaccccggccccaggggcgtgaagggcgccgacggcgtgaggggcctgaagggcagcaagggcgagaagggcgaggacggcttccccggcttcaagggcgacatgggcctgaagggcgacaggggcgaggtgggccagatcggccccaggggcgaggacggccccgagggccccaagggcagggccggccccaccggcgaccccggccccagcggccaggccggcgagaagggcaagctgggcgtgcccggcctgcccggctaccccggcaggcagggccccaagggcagcaccggcttccccggcttccccggcgccaacggcgagaagggcgccaggggcgtggccggcaagcccggccccaggggccagaggggccccaccggccccaggggcagcaggggcgccaggggccccaccggcaagcccggccccaagggcaccagcggcggcgacggcccccccggcccccccggcgagaggggcccccagggcccccagggccccgtgggcttccccggccccaagggcccccccggcccccccggcaaggacggcctgcccggccaccccggccagaggggcgagaccggcttccagggcaagaccggcccccccggccccggcggcgtggtgggcccccagggccccaccggcgagaccggccccatcggcgagaggggccaccccggcccccccggcccccccggcgagcagggcctgcccggcgccgccggcaaggagggcgccaagggcgaccccggcccccagggcatcagcggcaaggacggccccgccggcctgaggggcttccccggcgagaggggcctgcccggcgcccagggcgcccccggcctgaagggcggcgagggcccccagggcccccccggccccgtgggcagccccggcgagaggggcagcgccggcaccgccggccccatcggcctgcccggcaggcccggcccccagggcccccccggccccgccggcgagaagggcgcccccggcgagaagggcccccagggccccgccggcagggacggcgtgcagggccccgtgggcctgcccggccccgccggccccgccggcagccccggcgaggacggcgacaagggcgagatcggcgagcccggccagaagggcagcaagggcgacaagggcgagaacggcccccccggcccccccggcctgcagggccccgtgggcgcccccggcatcgccggcggcgacggcgagcccggccccaggggccagcagggcatgttcggccagaagggcgacgagggcgccaggggcttccccggcccccccggccccatcggcctgcagggcctgcccggcccccccggcgagaagggcgagaacggcgacgtgggccccatgggcccccccggcccccccggccccaggggcccccagggccccaacggcgccgacggcccccagggcccccccggcagcgtgggcagcgtgggcggcgtgggcgagaagggcgagcccggcgaggccggcaaccccggcccccccggcgaggccggcgtgggcggccccaagggcgagaggggcgagaagggcgaggccggcccccccggcgccgccggcccccccggcgccaagggcccccccggcgacgacggccccaagggcaaccccggccccgtgggcttccccggcgaccccggcccccccggcgagcccggccccgccggccaggacggcgtgggcggcgacaagggcgaggacggcgaccccggccagcccggcccccccggccccagcggcgaggccggcccccccggcccccccggcaagaggggcccccccggcgccgccggcgccgagggcaggcagggcgagaagggcgccaagggcgaggccggcgccgagggcccccccggcaagaccggccccgtgggcccccagggccccgccggcaagcccggccccgagggcctgaggggcatccccggccccgtgggcgagcagggcctgcccggcgccgccggccaggacggcccccccggccccatgggcccccccggcctgcccggcctgaagggcgaccccggcagcaagggcgagaagggccaccccggcctgatcggcctgatcggcccccccggcgagcagggcgagaagggcgacaggggcctgcccggcacccagggcagccccggcgccaagggcgacggcggcatccccggccccgccggccccctgggcccccccggcccccccggcctgcccggcccccagggccccaagggcaacaagggcagcaccggccccgccggccagaagggcgacagcggcctgcccggcccccccggcccccccggcccccccggcgaggtgatccagcccctgcccatcctgagcagcaagaagaccaggaggcacaccgagggcatgcaggccgacgccgacgacaacatcctggactacagcgacggcatggaggagatcttcggcagcctgaacagcctgaagcaggacatcgagcacatgaagttccccatgggcacccagaccaaccccgccaggacctgcaaggacctgcagctgagccaccccgacttccccgacggcgagtactggatcgaccccaaccagggctgcagcggcgacagcttcaaggtgtactgcaacttcaccagcggcggcgagacctgcatctaccccgacaagaagagcgagggcgtgaggatcagcagctggcccaaggagaagcccggcagctggttcagcgagttcaagaggggcaagctgctgagctacctggacgtggagggcaacagcatcaacatggtgcagatgaccttcctgaagctgctgaccgccagcgccaggcagaacttcacctaccactgccaccagagcgccgcctggtacgacgtgagcagcggcagctacgacaaggccctgaggttcctgggcagcaacgacgaggagatgagctacgacaacaaccccttcatcaagaccctgtacgacggctgcgccagcaggaagggctacgagaagaccgtgatcgagatcaacacccccaagatcgaccaggtgcccatcgtggacgtgatgatcaacgacttcggcgaccagaaccagaagttcggcttcgaggtgggccccgtgtgcttcctgggc

SEQ ID NO: 7 - DNA, 20 bp, Artificial Sequence
  Primer
gtgccctgactgaggctgtc

SEQ ID NO: 8 - DNA, 21 bp, Artificial Sequence
  Primer
ggatcatcgtgtcctgctcac

SEQ ID NO: 9 - DNA, 20 bp, Artificial Sequence
  Primer
gcatggccaagaagacatcc

SEQ ID NO: 10 - DNA, 19 bp, Artificial Sequence
  Primer
cctcgggtttccacgtctc

SEQ ID NO: 11 - DNA, 21 bp, Artificial Sequence
  Primer
aggctgagagtgtaacagaga

SEQ ID NO: 12 - DNA, 24 bp, Artificial Sequence
  Primer
tctgtttgtgctactgtttcttca

SEQ ID NO: 13 - DNA, 21 bp, Artificial Sequence
  Primer
gttcacatcccccaaatctga

SEQ ID NO: 14 - DNA, 20 bp, Artificial Sequence
  Primer
cccctagtttggctttggct

SEQ ID NO: 15 - DNA, 23 bp, Artificial Sequence
  Primer
ggaacaatggaaccttaccagac

SEQ ID NO: 16 - DNA, 20 bp, Artificial Sequence
  Primer
attcgatcctgatacccgcc

SEQ ID NO: 17 - DNA, 20 bp, Artificial Sequence
  Primer
aggagtagacggcagggatt

SEQ ID NO: 18 - DNA, 22 bp, Artificial Sequence
  Primer
ggaggtcgtagtcctttcttca

SEQ ID NO: 19 - DNA, 20 bp, Artificial Sequence
  Primer
ccgggagcagtgtgagctta

SEQ ID NO: 20 - DNA, 21 bp, Artificial Sequence
  Primer
tagatgcgtttgtaggcggtc

SEQ ID NO: 21 - DNA, 22 bp, Artificial Sequence
  Primer
cgcgtctccttcgagctgtttg

SEQ ID NO: 22 - DNA, 24 bp, Artificial Sequence
  Primer
tgtaaagtcaccaccctggcacat

SEQ ID NO: 23 - DNA, 20 bp, Artificial Sequence
  Primer
gtgcggtgcaaactttctcc

SEQ ID NO: 24 - DNA, 20 bp, Artificial Sequence
  Primer
aatgactcggttggtctcgg